(19) United States
(12) Patent Application Publication
(10) Pub. No.: US 2010/0137303 A1
(43) Pub. Date: Jun. 3, 2010

(54) NEW COMPOUND 255

(75) Inventors: Sandeep Raghunath Ghorpade, Bangalore (IN); Manoj Ganpat Kale, Bangalore (IN); Shahul Hameed Peer Mohamed, Bangalore (IN); Anand Kumar Venkobachar Raichurkar, Bangalore (IN); David Charles McKinney, Waltham, MA (US)

Correspondence Address: ASTRAZENECA R&D BOSTON, 35 GATEHOUSE DRIVE, WALTHAM, MA, 02451-1215, US

(73) Assignee: AstraZeneca AB

(21) Appl. No.: 12/476,418

(22) Filed: Jun. 2, 2009

Related U.S. Application Data
(60) Provisional application No. 61/058,748, filed on Jun. 4, 2008.

(51) Int. Cl. A61K 31/437; C07D 513/04; A61K 31/497; A61K 31/506; A61K 31/5377; A61P 31/04

(52) U.S. Cl. 514/234.2; 546/114; 544/316; 544/333; 544/127; 544/405; 514/301; 514/255.05; 514/274; 514/256

(57) ABSTRACT

Compounds of formula (I) and their pharmaceutically acceptable salts are described. Processes for their preparation, pharmaceutical compositions containing them, their use as medicaments and their use in the treatment of bacterial infections are also described.

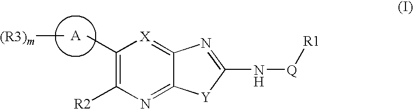

This application claims the benefit under 35 U.S.C. §119(e) of U.S. Provisional Application No. 61/058,748 filed on Jun. 4, 2008.

The present invention relates to compounds, which demonstrate antibacterial activity, processes for their preparation, pharmaceutical compositions containing them as the active ingredient, to their use as medicaments and to their use in the manufacture of medicaments for use in the treatment of bacterial infections in warm-blooded animals such as humans. In particular this invention relates to compounds useful for the treatment of bacterial infections in warm-blooded animals such as humans, more particularly to the use of these compounds in the manufacture of medicaments for use in the treatment of bacterial infections in warm-blooded animals such as humans.

The international microbiological community continues to express serious concern that the evolution of antibiotic resistance could result in strains against which currently available antibacterial agents will be ineffective. In general, bacterial pathogens may be classified as either Gram-positive or Gram-negative pathogens. Antibiotic compounds with effective activity against both Gram-positive and Gram-negative pathogens are generally regarded as having a broad spectrum of activity. The compounds of the present invention are regarded as effective against both Gram-positive and certain Gram-negative pathogens.

Gram-positive pathogens, for example Staphylococci, Enterococci, Streptococci and mycobacteria, are particularly important because of the development of resistant strains, which are both difficult to treat, and difficult to eradicate from the hospital environment once established. Examples of such strains are methicillin resistant staphylococcus aureus (MRSA), methicillin resistant coagulase negative staphylococci (MRCNS), penicillin resistant Streptococcus pneumoniae and multiple resistant Enterococcus faecium.

The preferred clinically effective antibiotic for treatment of last resort of such resistant Gram-positive pathogens is vancomycin. Vancomycin is a glycopeptide and is associated with various toxicities, including nephrotoxicity. Furthermore, and most importantly, antibacterial resistance to vancomycin and other glycopeptides is also appearing. This resistance is increasing at a steady rate rendering these agents less and less effective in the treatment of Gram-positive pathogens. There is also now increasing resistance appearing towards agents such as β-lactams, quinolones and macrolides used for the treatment of upper respiratory tract infections, also caused by certain Gram negative strains including H. influenzae and M. catarrhalis.

Consequently, in order to overcome the threat of widespread multi-drug resistant organisms, there is an on-going need to develop new antibiotics, particularly those with either a novel mechanism of action and/or containing new pharmacophoric groups.

Deoxyribonucleic acid (DNA) gyrase is a member of the type II family of topoisomerases that control the topological state of DNA in cells (Champoux, J. J.; 2001. Ann. Rev. Biochem. 70: 369-413). Type II topoisomerases use the free energy from adenosine triphosphate (ATP) hydrolysis to alter the topology of DNA by introducing transient double-stranded breaks in the DNA, catalyzing strand passage through the break and resealing the DNA. DNA gyrase is an essential and conserved enzyme in bacteria and is unique among topoisomerases in its ability to introduce negative supercoils into DNA. The enzyme consists of two subunits, encoded by gyrA and gyrB, forming an A₂B2 tetrameric complex. The A subunit of gyrase (GyrA) is involved in DNA breakage and resealing and contains a conserved tyrosine residue that forms the transient covalent link to DNA during strand passage. The B subunit (GyrB) catalyzes the hydrolysis of ATP and interacts with the A subunit to translate the free energy from hydrolysis to the conformational change in the enzyme that enables strand-passage and DNA resealing.

Another conserved and essential type II topoisomerase in bacteria, called topoisomerase IV, is primarily responsible for separating the linked closed circular bacterial chromosomes produced in replication. This enzyme is closely related to DNA gyrase and has a similar tetrameric structure formed from subunits homologous to Gyr A and to Gyr B. The overall sequence identity between gyrase and topoisomerase IV in different bacterial species is high. Therefore, compounds that target bacterial type II topoisomerases have the potential to inhibit two targets in cells, DNA gyrase and topoisomerase IV; as is the case for existing quinolone antibacterials (Maxwell, A. 1997, Trends Microbiol. 5: 102-109).

DNA gyrase is a well-validated target of antibacterials, including the quinolones and the coumarins. The quinolones (e.g. ciprofloxacin) are broad-spectrum antibacterials that inhibit the DNA breakage and reunion activity of the enzyme and trap the GyrA subunit covalently complexed with DNA (Drlica, K., and X. Zhao, 1997, Microbiol. Molec. Biol. Rev. 61: 377-392). Members of this class of antibacterials also inhibit topoisomerase IV and as a result, the primary target of these compounds varies among species. Although the quinolones are successful antibacterials, resistance generated by mutations in the target (DNA gyrase and topoisomerase IV) is becoming an increasing problem in several organisms, including S. aureus and Streptococcus pneumoniae (Hooper, D. C., 2002, The Lancet Infectious Diseases 2: 530-538). In addition, quinolones, as a chemical class, suffer from toxic side effects, including arthropathy that prevents their use in children (Lipsky, B. A. and Baker, C. A., 1999, Clin. Infect. Dis. 28: 352-364). Furthermore, the potential for cardiotoxicity, as predicted by prolongation of the QT_{c} interval, has been cited as a toxicity concern for quinolones.

There are several known natural product inhibitors of DNA gyrase that compete with ATP for binding the GyrB subunit (Maxwell, A. and Lawson, D. M. 2003, Curr. Topics in Med. Chem. 3: 283-303). The coumarins are natural products isolated from Streptomyces spp., examples of which are novobiocin, chlorobiocin and coumermycin A 1. Although these compounds are potent inhibitors of DNA gyrase, their therapeutic utility is limited due to toxicity in eukaryotes and poor penetration in Gram-negative bacteria (Maxwell, A. 1997, Trends Microbiol. 5: 102-109). Another natural product class of compounds that targets the GyrB subunit is the cyclothialidines, which are isolated from Streptomyces filipensis (Watanabe, J. et al 1994, J. Antibiot. 47: 32-36). Despite potent activity against DNA gyrase, cyclothialidine is a poor antibacterial agent showing activity only against some eubacterial species (Nakada, N, 1993, Antimicrob. Agents Chemother. 37: 2656-2661).

Synthetic inhibitors that target the B subunit of DNA gyrase and topoisomerase IV are known in the art. For example, coumarin-containing compounds are described in patent application number WO 99/35155, 5,6-bicyclic heteroaromatic compounds are described in patent application WO 02/060879, and pyrazole compounds are described in patent application WO 01/52845 (U.S. Pat. No. 6,608,087). AstraZeneca has also published certain applications describing anti-bacterial compounds: WO2005/026149, WO2006/087544, WO2006/087548, WO2006/087543, WO2006/092599 and WO2006/092608.

We have discovered a new class of compounds which are useful for inhibiting DNA gyrase.

According to the present invention there is provided a compound of formula (I):

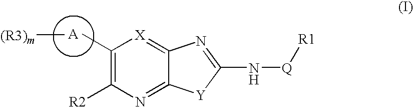

wherein

Y is S or O;

Q is C(═O)NR⁴, C(═S)NR⁵, C(═O)O, C(═NH)NR⁶, C(═NCN)NR⁷, SO₂NR⁸, C(═O)C(═O)NR⁹, or C═O, SO₂;

R⁴, R⁵, R⁶, R⁷, R⁸, R⁹ are independently selected from H, OH, C₁₋₄alkyl, and C₃₋₆ cycloalkyl;
R¹ is C₁₋₆alkyl, C₂₋₆alkenyl, C₂₋₆alkynyl, C₁₋₆alkoxy, C₁₋₆haloalkyl, C₁₋₆haloalkoxy, C₃₋₇ cycloalkyl, aryl, aryl C₁₋₆alkyl or heterocyclyl.

X is N or CRa wherein Ra is H, F, CH3, OCH3, CN;

m=0 to 5

Ring A is a carbocyclic or heterocyclic ring system comprising up to 12 ring atoms and up to 5 heteroatoms each independently selected from N, O and S; wherein if said heterocyclyl contains an —NH— moiety that nitrogen may be optionally substituted by a group R¹⁰);

R³ is hydrogen, halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N-(C₁₋₆alkyl)amino, N,N-(C₁₋₆alkyl)₂amino, C₁₋₆alkanoylamino, N-(C₁₋₆alkyl)carbamoyl, N,N-(C₁₋₆alkyl)₂carbamoyl, N-(C₁₋₆alkoxy)carbamoyl, N,N-(C₁₋₆alkoxy)₂carbamoyl, C₁₋₆alkylS(O)ₐ wherein a is 0 to 2, C₁₋₆alkoxycarbonyl, C₁₋₆alkoxycarbonylamino, N-(C₁₋₆alkyl)sulphamoyl, N,N-(C₁₋₆alkyl)₂sulphamoyl, C₁₋₆alkylsulphonylamino, carbocyclyl-R¹¹— or heterocyclyl-R¹²—; wherein R³ may be optionally substituted on carbon by one or more R¹³; and wherein if said heterocyclyl contains an —NH— moiety that nitrogen may be optionally substituted by a group selected from R¹⁴;

substituents on carbon are independently selected from halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N-(C₁₋₆alkyl)amino, N,N-(C₁₋₆alkyl)₂amino, C₁₋₆alkanoylamino, N-(C₁₋₆alkyl)carbamoyl, N,N-(C₁₋₆alkyl)₂carbamoyl, N-(C₁₋₆alkoxy)carbamoyl, N,N-(C₁₋₆alkoxy)₂carbamoyl, C₁₋₆alkylS(O)ₐ wherein a is 0 to 2, C₁₋₆alkoxycarbonyl, C₁₋₆alkoxycarbonylamino, N-(C₁₋₆alkyl)sulphamoyl, N,N-(C₁₋₆alkyl)₂sulphamoyl, C₁₋₆alkylsulphonylamino, carbocyclyl-R¹⁵— or heterocyclyl-R¹⁶—; and wherein if said heterocyclyl contains an —NH— moiety that nitrogen may be optionally substituted by a group selected from R¹⁷;

and wherein R³ may be directly attached to the C5 position of thiazolopyridine or oxazolopyrdine without ring A, in which case R³ is halogen, cyano, C₁₋₆alkyl, C₂₋₆alkenyl, C₂₋₆alkynyl, C₁₋₆alkoxy, C₁₋₆haloalkyl, C₁₋₆haloalkoxy, C₃₋₇ cycloalkyl, C₃₋₇ cycloalkoxy, N-(C₁₋₆alkyl)amino, N,N-(C₁₋₆alkyl)₂amino, N-(C₁₋₆alkyl)amino alkoxy, N,N-(C₁₋₆alkyl)₂amino alkoxy, heterocycloalkoxy with 1-5 heteroatoms in it, arylalkoxy, heterocycloalkyl, arylalkyl, N-(C₁₋₆alkyl)aminoalkoxy, N,N-(C₁₋₆alkyl)₂aminoalkoxy, C₁₋₆alkylS(O)ₐ wherein a is 0 to 2, C₁₋₆alkoxycarbonyl, C₁₋₆alkoxycarbonylamino, N-(C₁₋₆alkyl)sulphamoyl, N,N-(C₁₋₆alkyl)₂sulphamoyl, C₁₋₆alkylsulphonylamino.

R¹¹, R¹⁵ and R¹⁶ are independently selected from a direct bond, —O—, —N(R¹⁸)—, —C(O)—, —N(R¹⁹)C(O)—, —C(O)N(R²⁰)—, —S(O)ₛ—, —SO₂N(R²¹)— or —N(R²²)SO₂—; wherein R¹⁸, R¹⁹, R²⁰, R²¹ and R²² are independently selected from hydrogen or C₁₋₆alkyl and s is 0-2; and

R¹⁰, R¹⁴ and R¹⁷ are independently selected from C₁₋₆alkyl, C₃₋₆cycloalkyl, C₁₋₆alkanoyl, C₁₋₆alkylsulphonyl, C₁₋₆alkoxycarbonyl, carbamoyl, N-(C₁₋₆alkyl)carbamoyl, N,N-(C₁₋₆alkyl)carbamoyl, benzyl, benzyloxycarbonyl, benzoyl and phenylsulphonyl;

R¹³ and R¹² are independently selected from halo, nitro, cyano, hydroxy, trifluoromethoxy, trifluoromethyl, amino, carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl;

R² is H, C₁₋₆alkyl, C₂₋₆alkenyl, C₂₋₆alkynyl, C₁₋₆alkoxy, C₁₋₆haloalkyl, C₁₋₆haloalkoxy, C₃₋₇ cycloalkyl, C₃₋₇ cycloalkoxy, N-(C₁₋₆alkyl)amino, N,N-(C₁₋₆alkyl)₂amino, N-(C₁₋₆alkyl)amino alkoxy, N,N-(C₁₋₆alkyl)₂amino alkoxy, heterocycloalkoxy with 1-5 heteroatoms in it, arylalkoxy, heterocycloalkyl, arylalkyl, N-(C₁₋₆alkyl)aminoalkoxy, N,N-(C₁₋₆alkyl)₂aminoalkoxy, C₁₋₆alkylS(O)ₐ wherein a is 0 to 2, C₁₋₆alkoxycarbonyl, C₁₋₆alkoxycarbonylamino, N-(C₁₋₆alkyl)sulphamoyl, N,N-(C₁₋₆alkyl)₂sulphamoyl, or C₁₋₆alkylsulphonylamino, or

R² is a group

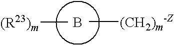

wherein

Z is O, S, or NR_{b} wherein R_{b} is H, C₁₋₆alkyl, C₃₋₇ cycloalkyl, C₁₋₆alkoxyC₁₋₆alkyl, cycloC₃₋₇alkoxyC₁₋₆alkyl; alternatively Z may represent a heterocyclic ring system comprising up to 7 ring atoms and up to 5 heteroatoms each independently selected from N, O and S,

alternatively Z is absent and the R² group is directly attached to the thiazolopyridine or oxazolopyridine ring at the C6 position,

Ring B is a carbocyclic or heterocyclic ring system comprising up to 12 ring atoms and up to 5 heteroatoms each independently selected from N, O and S; and wherein if said ring system contains an —NH— moiety that nitrogen may be optionally substituted by a group R¹⁰;

R²³ is hydrogen, halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N-(C₁₋₆alkyl)amino, N,N-(C₁₋₆alkyl)₂amino, C₁₋₆alkanoylamino, N-(C₁₋₆alkyl)carbamoyl, N,N-(C₁₋₆alkyl)₂carbamoyl, N-(C₁₋₆alkoxy)carbamoyl, N,N-(C₁₋₆alkoxy)₂carbamoyl, C₁₋₆alkylS(O)ₐ wherein a is 0 to 2, C₁₋₆alkoxycarbonyl, C₁₋₆alkoxycarbonylamino, N-(C₁₋₆alkyl)sulphamoyl, N,N-(C₁₋₆alkyl)₂sulphamoyl, C₁₋₆alkylsulphonylamino, carbocyclyl-R¹¹— or heterocyclyl-R¹²—; wherein the carbocyclyl or heterocyclyl may be optionally substituted on carbon by one or more R¹³; and wherein if said heterocyclyl contains an —NH— moiety that nitrogen may be optionally substituted by a group R¹⁴;

or alternatively Ring B may be absent and R²³ is directly attached to —(CH₂)ₘ—, in which case R²³ is selected from halogen, cyano, C₁₋₆alkyl, C₂₋₆alkenyl, C₂₋₆alkynyl, C₁₋₆alkoxy, C₁₋₆haloalkyl, C₁₋₆haloalkoxy, C₃₋₇ cycloalkyl, C₃₋₇ cycloalkoxy, N-(C₁₋₆alkyl)amino, N,N-(C₁₋₆alkyl)₂amino, N-(C₁₋₆alkyl)amino alkoxy, N,N-(C₁₋₆alkyl)₂amino alkoxy, heterocycloalkoxy with 1-5 heteroatoms in it, arylalkoxy, heterocycloalkyl, arylalkyl, N-(C₁₋₆alkyl)aminoalkoxy, N,N-(C₁₋₆alkyl)₂aminoalkoxy, C₁₋₆alkylS(O)ₐ wherein a is 0 to 2, C₁₋₆alkoxycarbonyl, C₁₋₆alkoxycarbonylamino, N-(C₁₋₆alkyl)sulphamoyl, N,N-(C₁₋₆alkyl)₂sulphamoyl, C₁₋₆alkylsulphonylamino;

or a pharmaceutically acceptable salt thereof.

In this specification the term alkyl includes both straight and branched chain alkyl groups. For example, “C₁₋₄alkyl” includes methyl, ethyl, propyl, isopropyl and t-butyl. However references to individual alkyl groups such as propyl are specific for the straight chain version only. An analogous convention applies to other generic terms.

Where optional substituents are chosen from one or more groups it is to be understood that this definition includes all substituents being chosen from one of the specified groups or the substituents being chosen from two or more of the specified groups.

A “heterocyclyl” is a saturated, partially saturated or unsaturated, mono or bicyclic ring containing 4-12 atoms of which at least one atom is chosen from nitrogen, sulphur or oxygen, which may, unless otherwise specified, be carbon or nitrogen linked, wherein a —CH₂— group can optionally be replaced by a —C(O)— and a ring sulphur atom may be optionally oxidised to form the S-oxide(s). In one aspect of the invention a “heterocyclyl” is a saturated, partially saturated or unsaturated, monocyclic ring containing 5 or 6 atoms of which at least one atom is chosen from nitrogen, sulphur or oxygen, it may, unless otherwise specified, be carbon or nitrogen linked, a —CH₂— group can optionally be replaced by a —C(O)— and a ring sulphur atom may be optionally oxidised to form the S-oxides. In a further aspect of the invention a “heterocyclyl” is an unsaturated, carbon-linked, monocyclic ring containing 5 or 6 atoms of which at least one atom is chosen from nitrogen, sulphur or oxygen. Examples and suitable values of the term “heterocyclyl” are morpholino, piperidyl, pyridyl, pyranyl, pyrrolyl, pyrazolyl, isothiazolyl, indolyl, quinolyl, thienyl, 1,3-benzodioxolyl, thiadiazolyl, piperazinyl, thiazolidinyl, pyrrolidinyl, thiomorpholino, pyrrolinyl, homopiperazinyl, 3,5-dioxapiperidinyl, tetrahydropyranyl, imidazolyl, pyrimidyl, pyrazinyl, pyridazinyl, isoxazolyl, N-methylpyrrolyl, 4-pyridone, 1-isoquinolone, 2-pyrrolidone, 4-thiazolidone, pyridine-N-oxide and quinoline-N-oxide. Further examples and suitable values of the term “heterocyclyl” are imidazolyl, 1,2,4-oxadiazolyl, 1,3,4-oxadiazolyl, pyrazolyl, 1,2,4-triazolyl, pyridyl, benzothiazolyl, isoxazolyl, pyrazinyl, pyrimidinyl and thiazolyl.

A “carbocyclyl” is a saturated, partially saturated or unsaturated, mono or bicyclic carbon ring that contains 3-12 atoms; wherein a —CH₂— group can optionally be replaced by a —C(O)—. Particularly “carbocyclyl” is a monocyclic ring containing 5 or 6 atoms or a bicyclic ring containing 9 or 10 atoms. Suitable values for “carbocyclyl” include cyclopropyl, cyclobutyl, 1-oxocyclopentyl, cyclopentyl, cyclopentenyl, cyclohexyl, cyclohexenyl, phenyl, naphthyl, tetralinyl, indanyl or 1-oxoindanyl. A particular example of “carbocyclyl” is phenyl.

An example of “C₁₋₄alkanoyloxy” is acetoxy. Examples of “C₁₋₄alkoxycarbonyl” are methoxycarbonyl, ethoxycarbonyl, n- and t-butoxycarbonyl. Examples of “C₁₋₄alkoxycarbonylamino” are methoxycarbonylamino, ethoxycarbonylamino, n- and is t-butoxycarbonylamino. Examples of “C₁₋₄alkoxy” are methoxy, ethoxy and propoxy. Examples of “C₁₋₄alkanoylamino” are formamido, acetamido and propionylamino. Examples of “C₁₋₄alkylS(O)ₐ wherein a is 0 to 2” are methylthio, ethylthio, methylsulphinyl, ethylsulphinyl, mesyl and ethylsulphonyl. Examples of “C₁₋₄alkanoyl” are propionyl and acetyl. Examples of “N-(C₁₋₄alkyl)₂amino” are methylamino and ethylamino. Examples of “N,N-(C₁₋₄alkyl)₂amino” are di-N-methylamino, di-(N-ethyl)amino and N-ethyl-N-methylamino. Examples of “C₂₋₄alkenyl” are vinyl, allyl and 1-propenyl. Examples of “C₂₋₄alkynyl” are ethynyl, 1-propynyl and 2-propynyl. Examples of “N-(C₁₋₄alkyl)sulphamoyl” are N-(methyl)sulphamoyl and N-(ethyl)sulphamoyl. Examples of “N,N-(C₁₋₄alkyl)₂sulphamoyl” are N,N-(dimethyl)sulphamoyl and N-(methyl)-N-(ethyl)sulphamoyl. Examples of “N-(C₁₋₄alkyl)carbamoyl” are methylaminocarbonyl and ethylaminocarbonyl. Examples of “N,N-(C₁₋₄alkyl)₂carbamoyl” are dimethylaminocarbonyl and methylethylaminocarbonyl. Examples of “N-(C₁₋₄alkoxy)carbamoyl” are methoxyaminocarbonyl and isopropoxyaminocarbonyl. Examples of “N-(C₁₋₄alkyl)-N-(C₁₋₄alkoxy)carbamoyl” are N-methyl-N-methoxyaminocarbonyl and N-methyl-N-ethoxyaminocarbonyl. Examples of “N′-(C₁₋₄alkyl)ureido” are N′-methylureido and N′-isopropylureido. Examples of “N′,N′-(C₁₋₄alkyl)₂ureido” are N′N′-dimethylureido and N′-methyl-N′-isopropylureido. Examples of “N′-(C₁₋₄alkyl)hydrazinocarbonyl” are N′-methylhydrazinocarbonyl and N′-isopropylhydrazinocarbonyl. Examples of “N′,N′-(C₁₋₄alkyl)₂hydrazinocarbonyl” are N′N′-dimethylhydrazinocarbonyl and N′-methyl-N′-isopropylhydrazinocarbonyl. Examples of “C₁₋₄alkylsulphonylamino” are methylsulphonylamino, isopropylsulphonylamino and t-butylsulphonylamino. Examples of “C₁₋₄alkylsulphonylaminocarbonyl” are methylsulphonylaminocarbonyl, isopropylsulphonylaminocarbonyl and t-butylsulphonylaminocarbonyl. Examples of “C₁₋₄alkylsulphonyl” are methylsulphonyl, isopropylsulphonyl and t-butylsulphonyl.

A compound of formula (I) may form stable acid or basic salts, and in such cases administration of a compound as a salt may be appropriate, and pharmaceutically acceptable salts may be made by conventional methods such as those described following.

Suitable pharmaceutically-acceptable salts include acid addition salts such as methanesulfonate, tosylate, α-glycerophosphate, fumarate, hydrochloride, citrate, maleate, tartrate and hydrobromide. Also suitable are salts formed with phosphoric and sulfuric acid. In another aspect suitable salts are base salts such as an alkali metal salt for example sodium, an alkaline earth metal salt for example calcium or magnesium, an organic amine salt for example triethylamine, morpholine, N-methylpiperidine, N-ethylpiperidine, procaine, dibenzylamine, N,N-dibenzylethylamine, tris-(2-hydroxyethyl)amine, N-methyl d-glucamine and amino acids such as lysine. There may be more than one cation or anion depending on the number of charged functions and the valency of the cations or anions. In one aspect of the invention the pharmaceutically-acceptable salt is the sodium salt.

However, to facilitate isolation of the salt during preparation, salts which are less soluble in the chosen solvent may be utilised whether pharmaceutically-acceptable or not.

Within the present invention it is to be understood that a compound of the formula (I) or a salt thereof may exhibit the phenomenon of tautomerism and that the formulae drawings within this specification can represent only one of the possible tautomeric forms. It is to be understood that the invention encompasses any tautomeric form which inhibits DNA gyrase and/or topoisomerase IV and is not to be limited merely to any one tautomeric form utilised within the formulae drawings. The formulae drawings within this specification can represent only one of the possible tautomeric forms and it is to be understood that the specification encompasses all possible tautomeric forms of the compounds drawn not just those forms which it has been possible to show graphically herein. The same applies to compound names.

It will be appreciated by those skilled in the art that compounds of formula (I) may contain asymmetrically substituted carbon(s) and sulphur atom(s), and accordingly may exist in, and be isolated in, as far as those additional asymmetrically substituted carbon(s) and sulphur atom(s) are concerned, optically-active and racemic forms at those positions. It is to be understood that the present invention encompasses any racemic, optically-active, polymorphic or stereoisomeric form, or mixtures thereof, at any additional asymmetrically substituted carbon(s) and sulphur atom(s), which possesses properties useful in the inhibition of DNA gyrase and/or topoisomerase IV.

Optically-active forms may be prepared by procedures known in the art for example, by resolution of the racemic form by recrystallization techniques, by synthesis from optically-active starting materials, by chiral synthesis, by enzymatic resolution, by biotransformation, or by chromatographic separation using a chiral stationary phase.

Some compounds may exhibit polymorphism. It is to be understood that the present invention encompasses any polymorphic form, or mixtures thereof, which form possesses properties useful in the inhibition of DNA gyrase and/or topoisomerase IV

It is also to be understood that certain compounds of the formula (I) and salts thereof can exist in solvated as well as unsolvated forms such as, for example, hydrated forms. It is to be understood that the invention encompasses all such solvated forms which inhibit DNA gyrase and/or topoisomerase IV.

There follow particular and suitable values for certain substituents and groups referred to in this specification. These values may be used where appropriate with any of the definitions and embodiments disclosed hereinbefore, or hereinafter. For the avoidance of doubt each stated species and any combination of species represents a particular and independent aspect of this invention.

Y is S or 0

Q is C(═O)NH, C(═S)NH, CO, C(═O)C(═O)NH

R¹ is —CH₃, CH₂CH₃, CH(CH3)₂, CH₂CH(CH3)₂, OCH₃, CF₃CH₂, CH₂CH═CH₂. cyclopropyl, prolinyl, pyrazinyl, pyrimidinyl

X represents CH, CF, N, CCH3, CCN, COCH3
Ring A is

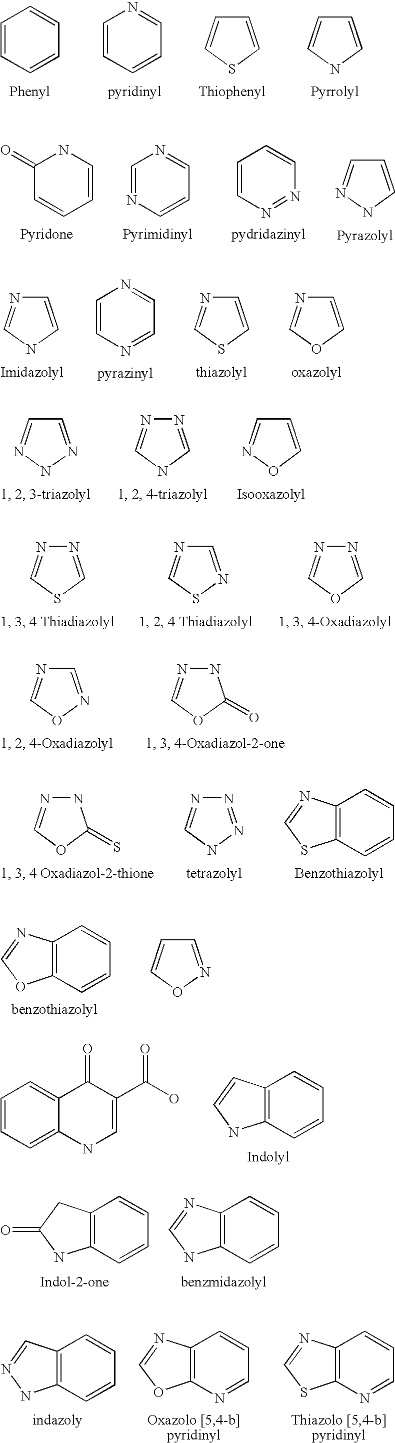

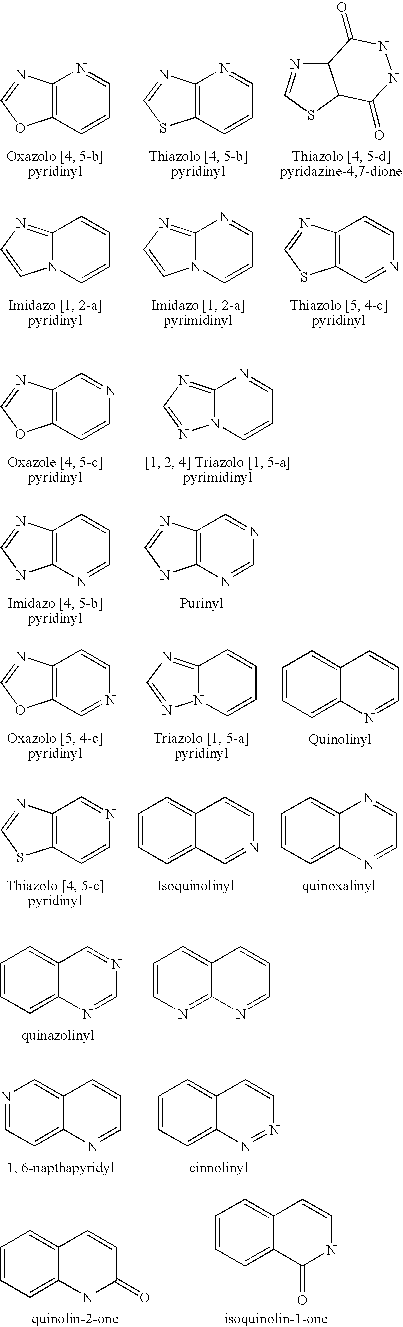

where R³ is attached to ring A, then it is selected from H, CH₃, CH₂CH₃, CH₂CF₃, OCH₃, OCH₂CH₃, Cl, Br, F, CN, CF₃, CHF₂, OCF₃, CH₂OCH₂CH₃, CH₂OCH₂CH₂OCH₃, CH₂OCH₂CF₃, OCH2CH2═CH₂, CONH₂, COOH, SO₂NH₂, NHCH₃, NHSO₂CH₃, NHSO₂CF₃, NHCOCH₃, NHCOCF₃, CONHCH₃, CONHCH₂CH₃, COCH₃,

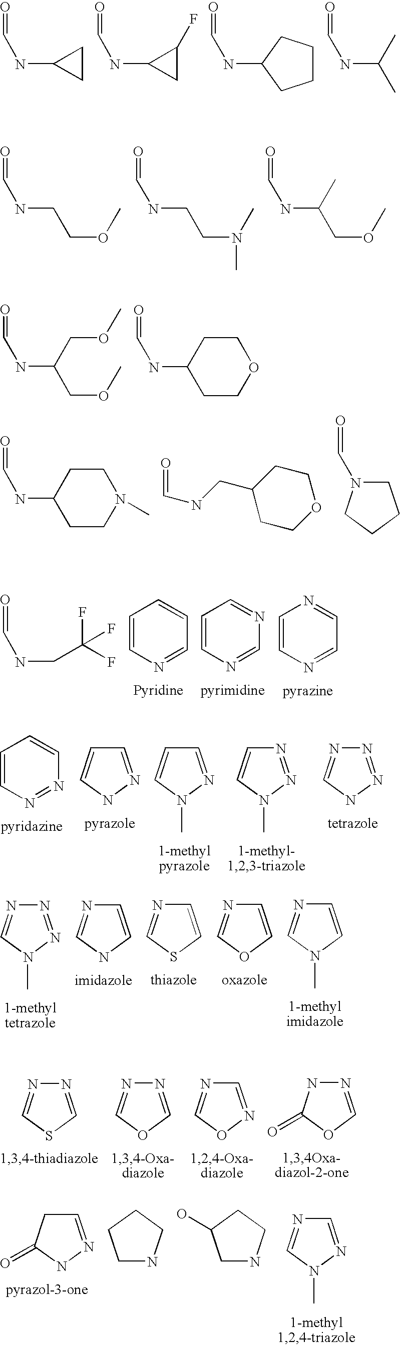

and where R³ is directly attached to thiazolopyridine or oxazolopyridine at the C5 position without ring A then R² is Cl, Br, CN, or CF₃

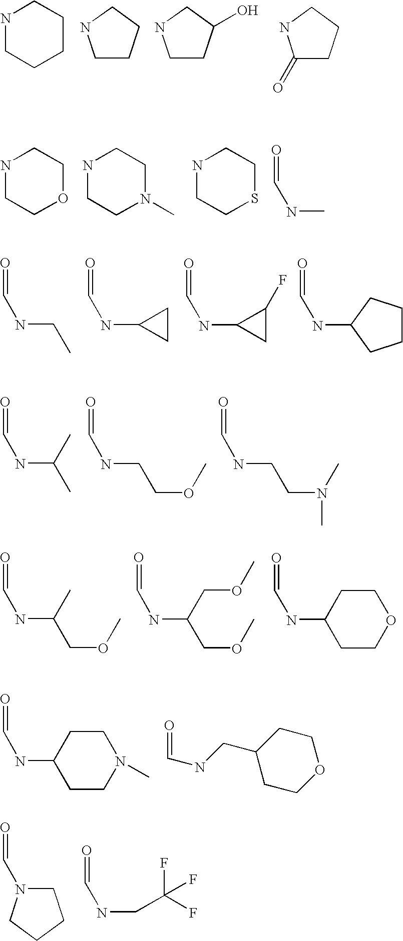

and pharmaceutically acceptable salts thereof.

R² is H, CH3, OCH3, OCH₂CH₃, OCF₃, OCH₂CH2═CH₂, OCH₂CF₃

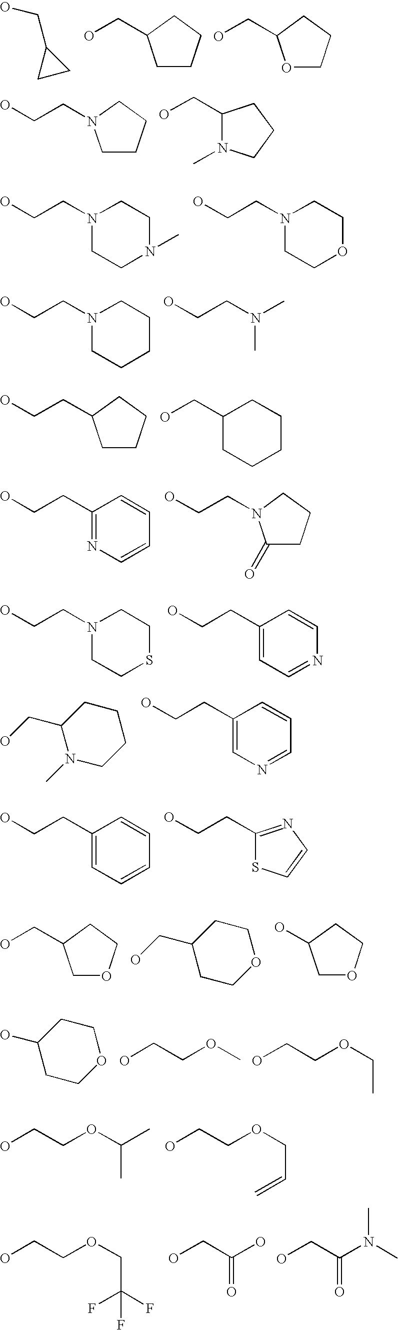

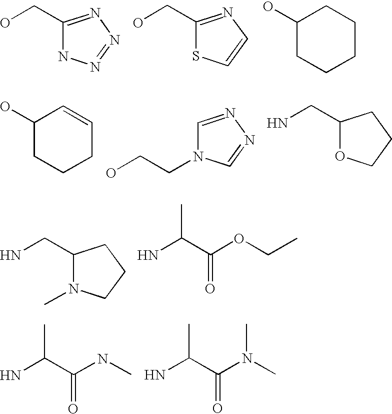

when R² is represented as

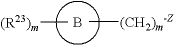

Z is O, S, NR_{b} wherein R_{b} is H, CH3, C2H5, CF3, CH2CH2OCH3, optionally N may be part of a heterocyclic ring such as piperidine, piperazine, morpholine, pyrrole, pyrazole, imidazole, triazole, tetrazole;

alternatively Z is absent and the R² group is directly attached to the thiazolopyridine or oxazolopyridine ring at the ‘C6 position

Ring B is

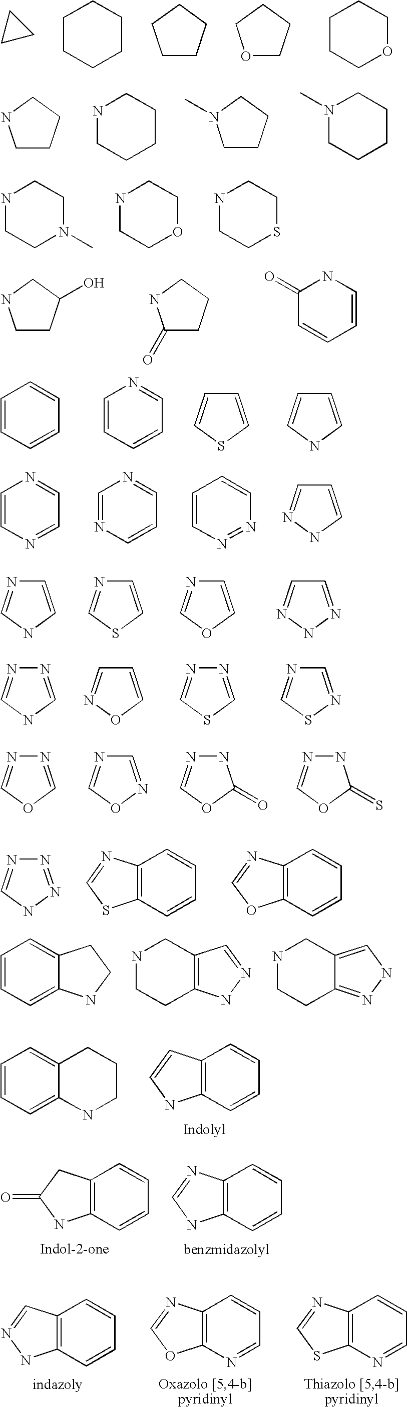

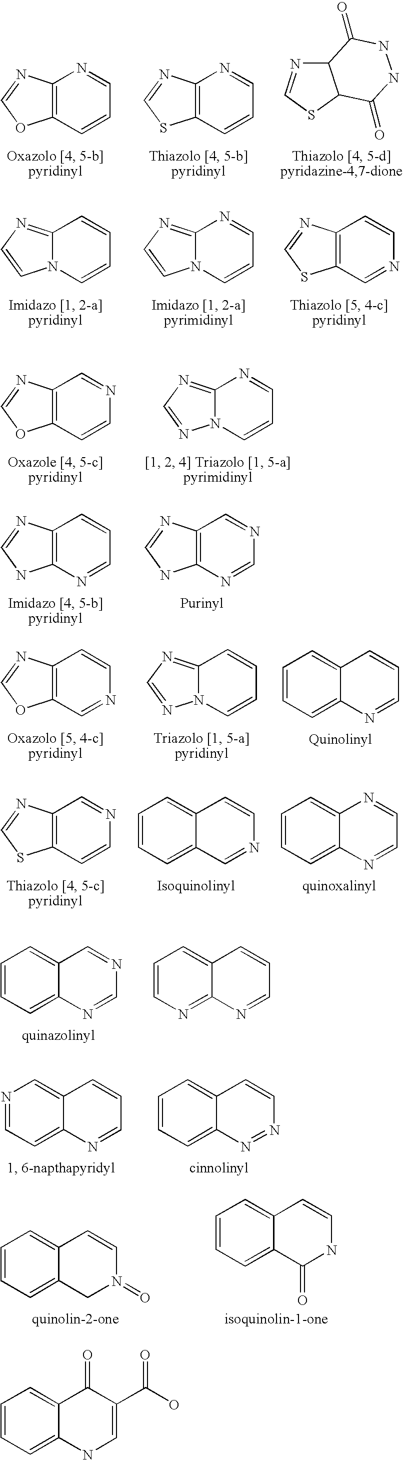

where R²³ is attached to ring B, then it is selected from H, CH₃, CH₂CH₃, CH₂CF₃, OCH₃, OCH₂CH₃, Cl, Br, F, CN, CF₃, CHF₂, OCF₃, OC(CH₃)₂, OCH₂CF₃, OCH₂CH═CH₂, CH₂OCH₂CH₃, CH₂OCH₂CH₂OCH₃, CH₂OCH₂CF₃, OC(CH₃)₂, OCH₂CF₃, CONH₂, COOH, SO₂NH₂, NHCH₃, NHSO₂CH₃, NHSO₂CF₃, NHCOCH₃, NHCOCF₃, CONHCH₃, CONHCH₂CH₃, COCH₃, COCH₂OH, COCH₂OCH₃

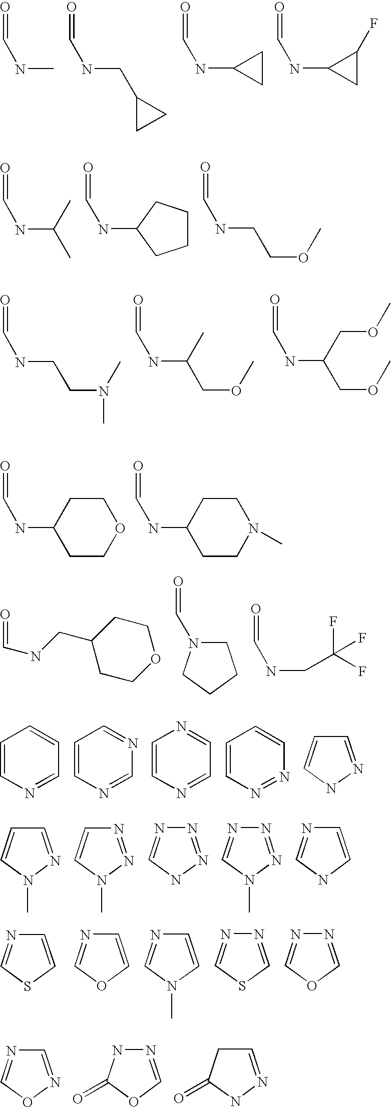

and where R²³ is directly attached to the —(CH₂)ₙ— linker i.e. ring B is absent, then R²³ is Cl, Br, F, CN, CF₃, OCH₃, OCH₂CH₃, OCF3, OC(CH3)2, OCH2CF3, OCH2CH═CH₂

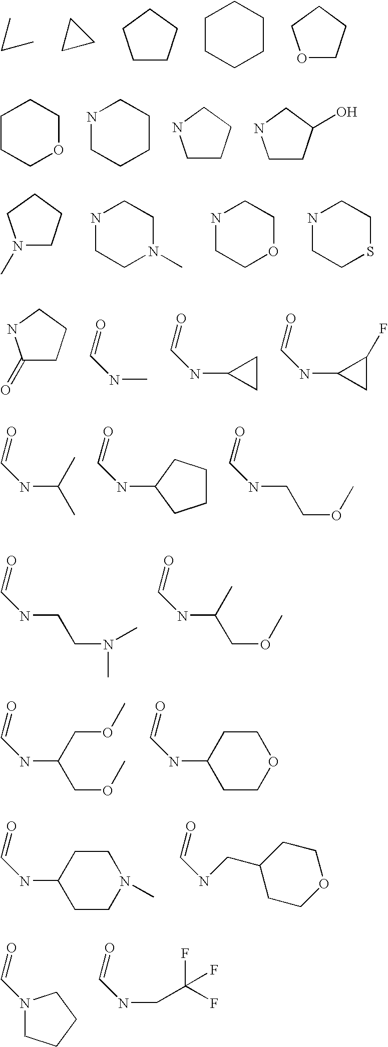

and pharmaceutically acceptable salts thereof.

Therefore in a further aspect of the invention there are provided compounds of formula (I) (as depicted above) wherein:

Y is S or O;

Q is C(═O)NH;

R¹ is —CH₃, CH₂CH₃, CH(CH₃)₂, CF₃CH₂, CH₂CH═CH₂;

X is CH;

m is 0-5

Ring A is selected from one of

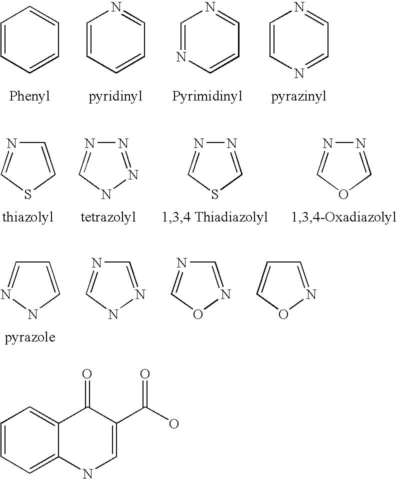

R³ is H, F, OCH₃, CH₃, CF₃, CHF₂, CN, CH₂OCH₂CH₃, CONH₂, COOH, Cl, COCH₃

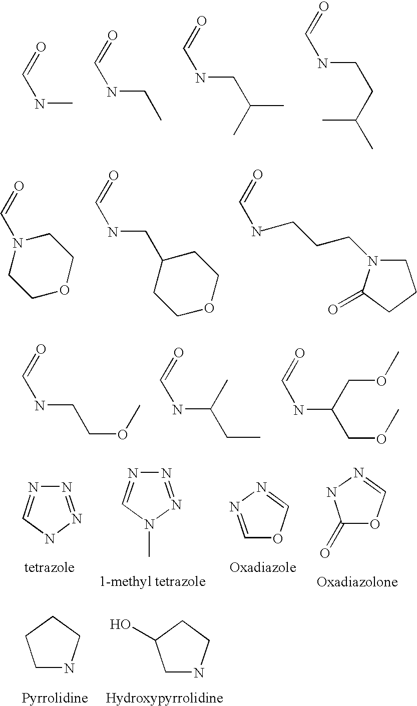

and pharmaceutically acceptable salts thereof.

R² is H, CH₃, OCH₃, OCH₂CH₃

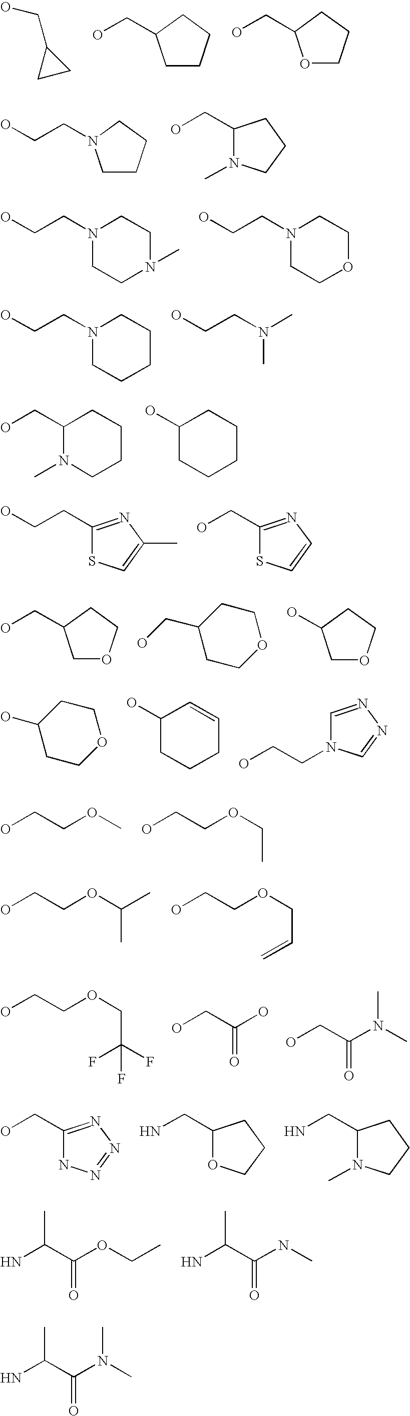

when R² is represented as

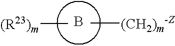

then Z is O, NH, or NCH₃, and optionally N is part of a heterocyclic ring such as piperidine, piperazine, morpholine, pyrazole, imidazole, triazole, tetrazole;

alternatively Z may be absent and the R² group is directly attached to the thiazolopyridine or oxazolopyridine ring at the C6 position

Ring B is selected from one of

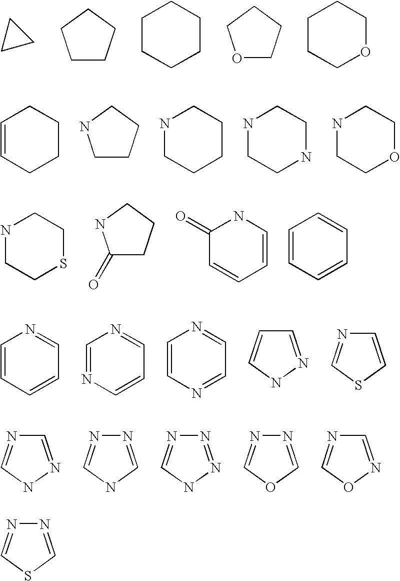

R²³ is H, F, OCH₃, OC2H5, OC(CH3)2, OCH2CH═CH2, OCH2CF3, CH₃, CF₃, CHF₂, CH₂OCH₂CH₃, CONH₂, COOH, Cl, COCH₃

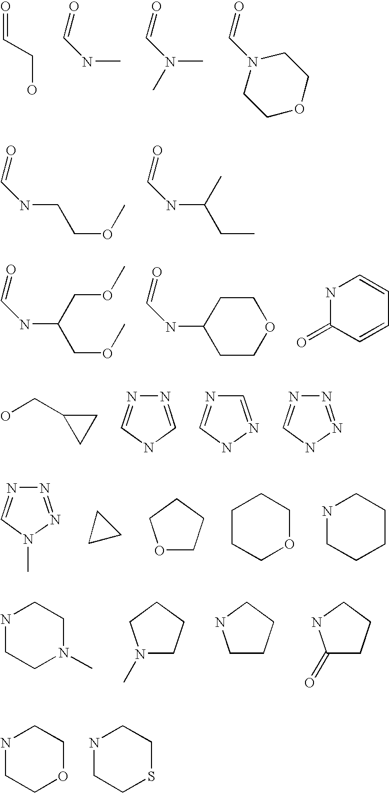

and pharmaceutically acceptable salts thereof.

Particular compounds of the invention are the compounds of the Examples, each of which provides a further independent aspect of the invention. In further aspects, the present invention also comprises any two or more compounds of the Examples or indeed any combination of the Examples of the invention.

In one embodiment of the invention are provided compounds of formula (I), in an alternative embodiment are provided pharmaceutically-acceptable salts of compounds of formula (I).

Another aspect of the present invention provides a process for preparing a compound of formula (I) or a pharmaceutically acceptable salt thereof (wherein R¹, R² are as defined in relation to formula I), which process comprises:

a. reacting an amine of the formula (IIa and IIb):

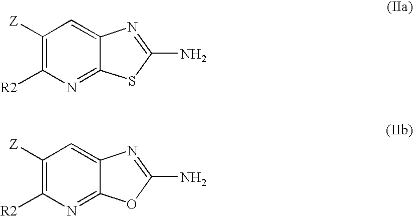

with isocyanate of formula (IIIa) or an activated derivative of formula (IIIb) to give formula IVa or IVb in presence suitable base and solvents.
wherein Z is a halogen;

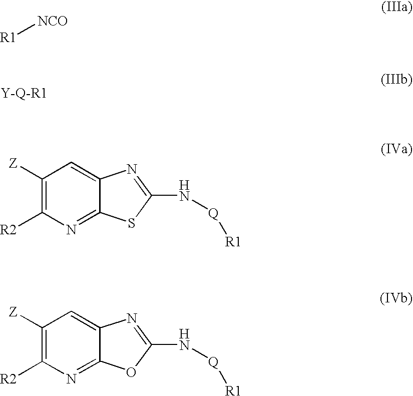

wherein Y is a displaceable group;

Suitable bases include triethylamine, di-isopropylethylamine, pyridine, or 2,6-di-alkyl-pyridines such as 2,6-lutidine or 2,6-di-tert-butylpyridine. Suitable solvents include dimethylacetamide, dichloromethane, N-methylpyrrolidone, tetrahydrofuran and dimethylformamide. The coupling reaction may conveniently be performed at a temperature in the range of 0° C. to 40° C.

Suitable activated derivatives of formula (IIIb) include active esters, for example pentafluorophenyl esters, acid halides, for example acid chlorides, and sulfonychlorides. The reaction of these types of compounds with amines is well known in the art, for example they may be reacted in the presence of a base, such as those described above, and in a suitable solvent, such as those described above. The reaction may conveniently be performed at a temperature in the range of 0° C. to 40° C.;

b) Reacting boronic acid or boronate ester of the formula (V)

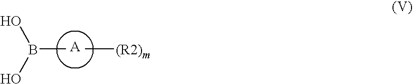

wherein R³, A, R⁷, n and m are as defined in relation to Formula I,
with a compound of formula (IVa) or (IVb) in the presence of a suitable palladium (0) catalyst to give a compound of formula I,
and
after process a) or b) above, if necessary doing one or more of the following:
i) converting a compound of the formula (I) into another compound of the formula (I);
ii) removing any protecting groups;
iii) forming a pharmaceutically acceptable salt.

The displaceable group X is conveniently selected from a halogen such as for example, a chloro, bromo or iodo group.

Compounds of formula (IIa and IIb) are commercially available, or known in the art, or may be made by processes known in the art.

Compounds of formula (IIIa and IIIb) are commercially available, or known in the art, or may be made by processes known in the art.

Compounds of formula (V) are commercially available, or known in the art, or may be made by processes known in the art.

The formation of a pharmaceutically-acceptable salt is within the skill of an ordinary organic chemist using standard techniques.

It will be appreciated that certain of the various ring substituents in the compounds of the present invention may be introduced by standard aromatic substitution reactions or generated by conventional functional group modifications either prior to or immediately following the processes mentioned above, and as such are included in the process aspect of the invention. The reagents used to introduce such ring substituents are either commercially available or are made by processes known in the art.

Introduction of substituents into a ring may convert one compound of the formula (I) into another compound of the formula (I). Such reactions and modifications include, for example, introduction of a substituent by means of an aromatic substitution reaction, reduction of substituents, alkylation of substituents, oxidation of substituents, esterification of substituents, amidation of substituents, formation of heteroaryl rings. The reagents and reaction conditions for such procedures are well known in the chemical art. Particular examples of aromatic substitution reactions include the introduction of alkoxides, diazotization reactions followed by introduction of thiol group, alcohol group, halogen group. Examples of modifications include; oxidation of alkylthio to alkylsulphinyl or alkylsulphonyl.

The skilled organic chemist will be able to use and adapt the information contained and referenced within the above references, and accompanying Examples therein and also the Examples herein, to obtain necessary starting materials, and products. If not commercially available, the necessary starting materials for the procedures such as those described above may be made by procedures which are selected from standard organic chemical techniques, techniques which are analogous to the synthesis of known, structurally similar compounds, or techniques which are analogous to the above described procedure or the procedures described in the examples. It is noted that many of the starting materials for synthetic methods as described above are commercially available and/or widely reported in the scientific literature, or could be made from commercially available compounds using adaptations of processes reported in the scientific literature. The reader is further referred to Advanced Organic Chemistry, 4^{th} Edition, by Jerry March, published by John Wiley & Sons 1992, for general guidance on reaction conditions and reagents.

It will also be appreciated that in some of the reactions mentioned herein it may be necessary/desirable to protect any sensitive groups in compounds. The instances where protection is necessary or desirable are known to those skilled in the art, as are suitable methods for such protection. Conventional protecting groups may be used in accordance with standard practice (for illustration see T. W. Greene, Protective Groups in Organic Synthesis, John Wiley and Sons, 1991).

Examples of a suitable protecting group for a hydroxy group is, for example, an acyl group, for example an alkanoyl group such as acetyl, an aroyl group, for example benzoyl, a silyl group such as trimethylsilyl or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively a silyl group such as trimethylsilyl may be removed, for example, by fluoride or by aqueous acid; or an arylmethyl group such as a benzyl group may be removed, for example, by hydrogenation in the presence of a catalyst such as palladium-on-carbon.

A suitable protecting group for an amino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The is deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a t-butoxycarbonyl group may be removed, for example, by treatment with a suitable acid as hydrochloric, sulphuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine or 2-hydroxyethylamine, or with hydrazine.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon, or for example, an allyl group which may be removed, for example, by use of a palladium catalyst such as palladium acetate.

The protecting groups may be removed at any convenient stage in the synthesis using conventional techniques well known in the chemical art, or they may be removed during a later reaction step or work-up.

Optically active forms of a compound of the invention may be obtained by carrying out one of the above procedures using an optically active starting material (formed, for example, by asymmetric induction of a suitable reaction step), or by resolution of a racemic form of the compound or intermediate using a standard procedure, or by chromatographic separation of diastereoisomers (when produced). Enzymatic techniques may also be useful for the preparation of optically active compounds and/or intermediates.

Similarly, when a pure regioisomer of a compound of the invention is required, it may be obtained by carrying out one of the above procedures using a pure regioisomer as a starting material, or by resolution of a mixture of the regioisomers or intermediates using a standard procedure.

According to a further feature of the invention there is provided a compound of the formula (I), or a pharmaceutically-acceptable salt thereof for use in a method of treatment of the human or animal body by therapy.

We have found that compounds of the present invention inhibit bacterial DNA gyrase and/or topoisomerase IV and are therefore of interest for their antibacterial effects. In one aspect of the invention the compounds of the invention inhibit bacterial DNA gyrase and are therefore of interest for their antibacterial effects. In one aspect of the invention the compounds of the invention inhibit topoisomerase IV and are therefore of interest for their antibacterial effects. In one aspect of the invention the compounds of the invention inhibit both DNA gyrase and topoisomerase IV and are therefore of interest for their antibacterial effects.

It is expected that the compounds of the present invention will be useful in treating bacterial infections. In one aspect of the invention “infection” or “bacterial infection” refers to a gynecological infection. In one aspect of the invention “infection” or “bacterial infection” refers to a respiratory tract infection (RTI). In one aspect of the invention “infection” or “bacterial infection” refers to a sexually transmitted disease. In one aspect of the invention “infection” or “bacterial infection” refers to a urinary tract infection. In one aspect of the invention “infection” or “bacterial infection” refers to acute exacerbation of chronic bronchitis (ACEB). In one aspect of the invention “infection” or “bacterial infection” refers to acute otitis media. In one aspect of the invention “infection” or “bacterial infection” refers to acute sinusitis. In one aspect of the invention “infection” or “bacterial infection” refers to an infection caused by drug resistant bacteria. In one aspect of the invention “infection” or “bacterial infection” refers to catheter-related sepsis. In one aspect of the invention “infection” or “bacterial infection” refers to chancroid. In one aspect of the invention “infection” or “bacterial infection” refers to chlamydia. In one aspect of the invention “infection” or “bacterial infection” refers to community-acquired pneumonia (CAP). In one aspect of the invention “infection” or “bacterial infection” refers to complicated skin and skin structure infection. In one aspect of the invention “infection” or “bacterial infection” refers to uncomplicated skin and skin structure infection. In one aspect of the invention “infection” or “bacterial infection” refers to endocarditis. In one aspect of the invention “infection” or “bacterial infection” refers to febrile neutropenia. In one aspect of the invention “infection” or “bacterial infection” refers to gonococcal cervicitis. In one aspect of the invention “infection” or “bacterial infection” refers to gonococcal urethritis. In one aspect of the invention “infection” or “bacterial infection” refers to hospital-acquired pneumonia (HAP). In one aspect of the invention “infection” or “bacterial infection” refers to osteomyelitis. In one aspect of the invention “infection” or “bacterial infection” refers to sepsis. In one aspect of the invention “infection” or “bacterial infection” refers to syphilis.

In one aspect of the invention an “infection” or “bacterial infection” refers to an infection caused by Acinetobacter baumanii. In one aspect of the invention an “infection” or “bacterial infection” refers to an infection caused by Acinetobacter haemolyticus. In one aspect of the invention an “infection” or “bacterial infection” refers to an infection caused by Acinetobacter junii. In one aspect of the invention an “infection” or “bacterial infection” refers to an infection caused by Acinetobacter johnsonii. In one aspect of the invention an “infection” or “bacterial infection” refers to an infection caused by Acinetobacter lwoffi. In one aspect of the invention an “infection” or “bacterial infection” refers to an infection caused by Bacteroides bivius. In one aspect of the invention an “infection” or “bacterial infection” refers to an infection caused by Bacteroides fragilis. In one aspect of the invention an “infection” or “bacterial infection” refers to an infection caused by Burkholderia cepacia. In one aspect of the invention an “infection” or “bacterial infection” refers to an infection caused by Campylobacter jejuni. In one aspect of the invention an “infection” or “bacterial infection” refers to an infection caused by Chlamydia pneumoniae. In one aspect of the invention an “infection” or “bacterial infection” refers to an infection caused by Chlamydia urealyticus. In one aspect of the invention an “infection” or “bacterial infection” refers to an infection caused by Chlamydophila pneumoniae. In one aspect of the invention an “infection” or “bacterial infection” refers to an infection caused by Clostridium difficile. In one aspect of the invention an “infection” or “bacterial infection” refers to an infection caused by Enterobacter aerogenes. In one aspect of the invention an “infection” or “bacterial infection” refers to an infection caused by Enterobacter cloacae. In one aspect of the invention an “infection” or “bacterial infection” refers to an infection caused by Enterococcus faecalis. In one aspect of the invention an “infection” or “bacterial infection” refers to an infection caused by Enterococcus faecium. In one aspect of the invention an “infection” or “bacterial infection” refers to an infection caused by Escherichia coli. In one aspect of the invention an “infection” or “bacterial infection” refers to an infection caused by Gardnerella vaginalis. In one aspect of the invention an “infection” or “bacterial infection” refers to an infection caused by Haemophilus parainfluenzae. In one aspect of the invention an “infection” or “bacterial infection” refers to an infection caused by Haemophilus influenzae. In one aspect of the invention an “infection” or “bacterial infection” refers to an infection caused by Helicobacter pylori. In one aspect of the invention an “infection” or “bacterial infection” refers to an infection caused by Klebsiella pneumoniae. In one aspect of the invention an “infection” or “bacterial infection” refers to an infection caused by Legionella pneumophila. In one aspect of the invention an “infection” or “bacterial infection” refers to an infection caused by Methicillin-resistant Staphylococcus aureus. In one aspect of the invention an “infection” or “bacterial infection” refers to an infection caused by Methicillin-susceptible Staphylococcus aureus. In one aspect of the invention an “infection” or “bacterial infection” refers to an infection caused by Moraxella catarrhalis. In one aspect of the invention an “infection” or “bacterial infection” refers to an infection caused by Morganella morganii. In one aspect of the invention an “infection” or “bacterial infection” refers to an infection caused by Mycoplasma pneumoniae. In one aspect of the invention an “infection” or “bacterial infection” refers to an infection caused by Neisseria gonorrhoeae. In one aspect of the invention an “infection” or “bacterial' infection” refers to an infection caused by Penicillin-resistant Streptococcus pneumoniae. In one aspect of the invention an “infection” or “bacterial infection” refers to an infection caused by Penicillin-susceptible Streptococcus pneumoniae. In one aspect of the invention an “infection” or “bacterial infection” refers to an infection caused by Peptostreptococcus magnus. In one aspect of the invention an “infection” or “bacterial infection” refers to an infection caused by Peptostreptococcus micros. In one aspect of the invention an “infection” or “bacterial infection” refers to an infection caused by Peptostreptococcus anaerobius. In one aspect of the invention an “infection” or “bacterial infection” refers to an infection caused by Peptostreptococcus asaccharolyticus. In one aspect of the invention an “infection” or “bacterial infection” refers to an infection caused by Peptostreptococcus prevotii. In one aspect of the invention an “infection” or “bacterial infection” refers to an infection caused by Peptostreptococcus tetradius. In one aspect of the invention an “infection” or “bacterial infection” refers to an infection caused by Peptostreptococcus vaginalis. In one aspect of the invention an “infection” or “bacterial infection” refers to an infection caused by Proteus mirabilis. In one aspect of the invention an “infection” or “bacterial infection” refers to an infection caused by Pseudomonas aeruginosa. In one aspect of the invention an “infection” or “bacterial infection” refers to an infection caused by Quinolone-Resistant Staphylococcus aureus. In one aspect of the invention an “infection” or “bacterial infection” refers to an infection caused by Quinolone-Resistant Staphylococcus epidermis. In one aspect of the invention an “infection” or “bacterial infection” refers to an infection caused by Salmonella typhi. In one aspect of the invention an “infection” or “bacterial infection” refers to an infection caused by Salmonella paratyphi. In one aspect of the invention an “infection” or “bacterial infection” refers to an infection caused by Salmonella enteritidis. In one aspect of the invention an “infection” or “bacterial infection” refers to an infection caused by Salmonella typhimurium. In one aspect of the invention an “infection” or “bacterial infection” refers to an infection caused by Serratia marcescens. In one aspect of the invention an “infection” or “bacterial infection” refers to an infection caused by Staphylococcus aureus. In one aspect of the invention an “infection” or “bacterial infection” refers to an infection caused by Staphylococcus epidermidis. In one aspect of the invention an “infection” or “bacterial infection” refers to an infection caused by Staphylococcus saprophyticus. In one aspect of the invention an “infection” or “bacterial infection” refers to an infection caused by Streptococcus agalactiae. In one aspect of the invention an “infection” or “bacterial infection” refers to an infection caused by Streptococcus pneumoniae. In one aspect of the invention an “infection” or “bacterial infection” refers to an infection caused by Streptococcus pyogenes. In one aspect of the invention an “infection” or “bacterial infection” refers to an infection caused by Stenotrophomonas maltophilia. In one aspect of the invention an “infection” or “bacterial infection” refers to an infection caused by Ureaplasma urealyticum. In one aspect of the invention an “infection” or “bacterial infection” refers to an infection caused by Vancomycin-Resistant Enterococcus faecium. In one aspect of the invention an “infection” or “bacterial infection” refers to an infection caused by Vancomycin-Resistant Enterococcus faecalis. In one aspect of the invention an “infection” or “bacterial infection” refers to an infection caused by Vancomycin-Resistant Staphylococcus aureus. In one aspect of the invention an “infection” or “bacterial infection” refers to an infection caused by Vancomycin-Resistant Staphylococcus epidermis.

In one aspect of the invention an “infection” or “bacterial infection” refers to an infection caused by Acinetobacter spp. In one aspect of the invention an “infection” or “bacterial infection” refers to an infection caused by Bacteroides spp. In one aspect of the invention an “infection” or “bacterial infection” refers to an infection caused by Burkholderia spp. In one aspect of the invention an “infection” or “bacterial infection” refers to an infection caused by Campylobacter spp. In one aspect of the invention an “infection” or “bacterial infection” refers to an infection caused by Chlamydia spp. In one aspect of the invention an “infection” or “bacterial infection” refers to an infection caused by Chlamydophila spp. In one aspect of the invention an “infection” or “bacterial infection” refers to an infection caused by Clostridium spp. In one aspect of the invention an “infection” or “bacterial infection” refers to an infection caused by Enterobacter spp. In one aspect of the invention an “infection” or “bacterial infection” refers to an infection caused by Enterococcus spp. In one aspect of the invention an “infection” or “bacterial infection” refers to an infection caused by Escherichia spp. In one aspect of the invention an “infection” or “bacterial infection” refers to an infection caused by Gardnerella spp. In one aspect of the invention an “infection” or “bacterial infection” refers to an infection caused by Haemophilus spp. In one aspect of the invention an “infection” or “bacterial infection” refers to an infection caused by Helicobacter spp. In one aspect of the invention an “infection” or “bacterial infection” refers to an infection caused by Klebsiella spp. In one aspect of the invention an “infection” or “bacterial infection” refers to an infection caused by Legionella spp. In one aspect of the invention an “infection” or “bacterial infection” refers to an infection caused by Moraxella spp. In one aspect of the invention an “infection” or “bacterial infection” refers to an infection caused by Morganella spp. In one aspect of the invention an “infection” or “bacterial infection” refers to an infection caused by Mycoplasma spp. In one aspect of the invention an “infection” or “bacterial infection” refers to an infection caused by Neisseria spp. In one aspect of the invention an “infection” or “bacterial infection” refers to an infection caused by Peptostreptococcus spp. In one aspect of the invention an “infection” or “bacterial infection” refers to an infection caused by Proteus spp. In one aspect of the invention an “infection” or “bacterial infection” refers to an infection caused by Pseudomonas spp. In one aspect of the invention an “infection” or “bacterial infection” refers to an infection caused by Salmonella spp. In one aspect of the invention an “infection” or “bacterial infection” is refers to an infection caused by Serratia spp. In one aspect of the invention an “infection” or “bacterial infection” refers to an infection caused by Staphylococcus spp. In one aspect of the invention an “infection” or “bacterial infection” refers to an infection caused by Streptoccocus spp. In one aspect of the invention an “infection” or “bacterial infection” refers to an infection caused by Stenotrophomonas spp. In one aspect of the invention an “infection” or “bacterial infection” refers to an infection caused by Ureaplasma spp. In one aspect of the invention an “infection” or “bacterial infection” refers to an infection caused by aerobes. In one aspect of the invention an “infection” or “bacterial infection” refers to an infection caused by obligate anaerobes. In one aspect of the invention an “infection” or “bacterial infection” refers to an infection caused by facultative anaerobes. In one aspect of the invention an “infection” or “bacterial infection” refers to an infection caused by gram-positive bacteria. In one aspect of the invention an “infection” or “bacterial infection” refers to an infection caused by gram-negative bacteria. In one aspect of the invention an “infection” or “bacterial infection” refers to an infection caused by gram-variable bacteria. In one aspect of the invention an “infection” or “bacterial infection” refers to an infection caused by atypical respiratory pathogens.

According to a further feature of the present invention the “infection” or “bacterial infection” refers to an infection caused by a mycobacterium and in particular any one of Mycobacterium tuberculosis (Mtu), M. avium intracellulare (Mai) and M. ulcerans (Mul).

According to a further feature of the present invention there is provided a method for producing an antibacterial effect in a warm blooded animal, such as man, in need of such treatment, which comprises administering to said animal an effective amount of a compound of the present invention, or a pharmaceutically-acceptable salt thereof.

According to a further feature of the invention there is provided a method for inhibition of bacterial DNA gyrase and/or topoisomerase IV in a warm-blooded animal, such as a human being, in need of such treatment which comprises administering to said animal an effective amount of a compound of formula (I) or a pharmaceutically acceptable salt thereof as defined hereinbefore.

According to a further feature of the invention there is provided a method of treating a bacterial infection in a warm-blooded animal, such as a human being, in need of such treatment which comprises administering to said animal an effective amount of a compound of formula (I) or a pharmaceutically acceptable salt thereof as defined hereinbefore.

According to a further feature of the invention there is provided a method of treating a bacterial infection selected from a gynecological infection, a respiratory tract infection (RTI), a sexually transmitted disease, a urinary tract infection, acute exacerbation of chronic bronchitis (ACEB), acute otitis media, acute sinusitis, an infection caused by drug resistant bacteria, catheter-related sepsis, chancroid, chlamydia, community-acquired pneumonia (CAP), complicated skin and skin structure infection, uncomplicated skin and skin structure infection, endocarditis, febrile neutropenia, gonococcal cervicitis, gonococcal urethritis, hospital-acquired pneumonia (HAP), osteomyelitis, sepsis and for syphilis in a warm-blooded animal, such as a human being, in need of such treatment which comprises administering to said animal an effective amount of a compound of formula (I) or a pharmaceutically acceptable salt thereof as defined hereinbefore.

A further feature of the present invention is a compound of formula (I) and pharmaceutically acceptable salts thereof for use as a medicament. Suitably the medicament is an antibacterial agent.

According to a further aspect of the invention there is provided the use of a compound of formula (I), or a pharmaceutically acceptable salt thereof in the manufacture of a medicament for the production of an anti-bacterial effect in a warm-blooded animal such as a human being.

According to a further aspect of the invention there is provided the use of a compound of formula (I), or a pharmaceutically acceptable salt thereof in the manufacture of a medicament for the inhibition of bacterial DNA gyrase and/or topoisomerase IV in a warm-blooded animal such as a human being.

Thus according to a further aspect of the invention there is provided the use of a compound of formula (I), or a pharmaceutically acceptable salt thereof in the manufacture of a medicament for the treatment of a bacterial infection in a warm-blooded animal such as a human being.

According to a further feature of the invention there is provided a method of treating a bacterial infection selected from pulmonary tuberculosis, extra-pulmonary is tuberculosis, avium infections, Buruli ulcer in a warm-blooded animal, such as a human being, in need of such treatment which comprises administering to said animal an effective amount of a compound of formula (I) or a pharmaceutically acceptable salt thereof as defined hereinbefore.

Thus according to a further aspect of the invention there is provided the use of a compound of formula (I), or a pharmaceutically acceptable salt thereof in the manufacture of a medicament for the treatment of a bacterial infection selected from a gynecological infection, a respiratory tract infection (RTI), a sexually transmitted disease, a urinary tract infection, acute exacerbation of chronic bronchitis (ACEB), acute otitis media, acute sinusitis, an infection caused by drug resistant bacteria, catheter-related sepsis, chancroid, chlamydia, community-acquired pneumonia (CAP), complicated skin and skin structure infection, uncomplicated skin and skin structure infection, endocarditis, febrile neutropenia, gonococcal cervicitis, gonococcal urethritis, hospital-acquired pneumonia (HAP), osteomyelitis, sepsis and/or syphilis in a warm-blooded animal such as a human being.

According to a further aspect of the invention there is provided a compound of formula (I), or a pharmaceutically acceptable salt thereof for use in the production of an anti-bacterial effect in a warm-blooded animal such as a human being.

According to a further aspect of the invention there is provided a compound of formula (I), or a pharmaceutically acceptable salt thereof for use in inhibition of bacterial DNA gyrase and/or topoisomerase IV in a warm-blooded animal such as a human being.

Thus according to a further aspect of the invention there is provided a compound of formula (I), or a pharmaceutically acceptable salt thereof for use in the treatment of a bacterial infection in a warm-blooded animal such as a human being.

Thus according to a further aspect of the invention there is provided a compound of formula (I), or a pharmaceutically acceptable salt thereof for use in the treatment of a bacterial infection selected from a gynecological infection, a respiratory tract infection (RTI), a sexually transmitted disease, a urinary tract infection, acute exacerbation of chronic bronchitis (ACEB), acute otitis media, acute sinusitis, an infection caused by drug resistant bacteria, catheter-related sepsis, chancroid, chlamydia, community-acquired pneumonia (CAP), complicated skin and skin structure infection, uncomplicated skin and skin structure infection, endocarditis, febrile neutropenia, gonococcal cervicitis, is gonococcal urethritis, hospital-acquired pneumonia (HAP), osteomyelitis, sepsis and/or syphilis in a warm-blooded animal such as a human being.

In order to use a compound of the formula (I) or a pharmaceutically-acceptable salt thereof, for the therapeutic (including prophylactic) treatment of mammals including humans, in particular in treating infection, it is normally formulated in accordance with standard pharmaceutical practice as a pharmaceutical composition.

Therefore in another aspect the present invention provides a pharmaceutical composition which comprises a compound of the formula (I) or a pharmaceutically-acceptable salt thereof, and a pharmaceutically-acceptable diluent or carrier.

According to a further aspect of the invention there is provided a pharmaceutical composition which comprises a compound of formula (I) as defined hereinbefore or a pharmaceutically acceptable salt thereof, in association with a pharmaceutically acceptable excipient or carrier for use in producing an anti-bacterial effect in a warm-blooded animal, such as a human being.

According to a further aspect of the invention there is provided a pharmaceutical composition which comprises a compound of formula (I) as defined hereinbefore or a pharmaceutically acceptable salt thereof, in association with a pharmaceutically acceptable excipient or carrier for use in inhibition of bacterial DNA gyrase and/or topoisomerase IV in a warm-blooded animal, such as a human being.

According to a further aspect of the invention there is provided a pharmaceutical composition which comprises a compound of formula (I) as defined hereinbefore or a pharmaceutically acceptable salt thereof, in association with a pharmaceutically acceptable excipient or carrier for use in the treatment of a bacterial infection in a warm-blooded animal, such as a human being.

According to a further aspect of the invention there is provided a pharmaceutical composition which comprises a compound of formula (I) as defined hereinbefore or a pharmaceutically acceptable salt thereof, in association with a pharmaceutically acceptable excipient or carrier for use in the treatment of a gynecological infection, a respiratory tract infection (RTI), a sexually transmitted disease, a urinary tract infection, acute exacerbation of chronic bronchitis (ACEB), acute otitis media, acute sinusitis, an infection caused by drug resistant bacteria, catheter-related sepsis, chancroid, chlamydia, community-acquired pneumonia (CAP), complicated skin and skin structure infection, uncomplicated skin and skin structure infection, endocarditis, febrile neutropenia, gonococcal cervicitis, gonococcal urethritis, hospital-acquired pneumonia (HAP), osteomyelitis, sepsis and/or syphilis in a warm-blooded animal, such as a human being.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular or intramuscular dosing or as a suppository for rectal dosing).

The compositions of the invention may be obtained by conventional procedures using conventional pharmaceutical excipients, well known in the art. Thus, compositions intended for oral use may contain, for example, one or more colouring, sweetening, flavouring and/or preservative agents.

Suitable pharmaceutically acceptable excipients for a tablet formulation include, for example, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal tract, or to improve their stability and/or appearance, in either case, using conventional coating agents and procedures well known in the art.

Compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

Aqueous suspensions generally contain the active ingredient in finely powdered form together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as ethyl or propyl p-hydroxybenzoate, anti-oxidants (such as ascorbic acid), colouring agents, flavouring agents, and/or sweetening agents (such as sucrose, saccharine or aspartame).

Oily suspensions may be formulated by suspending the active ingredient in a vegetable oil (such as arachis oil, olive oil, sesame oil or coconut oil) or in a mineral oil (such as liquid paraffin). The oily suspensions may also contain a thickening agent such as beeswax, hard paraffin or cetyl alcohol. Sweetening agents such as those set out above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water generally contain the active ingredient together with a dispersing or wetting agent, suspending agent and one or more preservatives. Suitable dispersing or wetting agents and suspending agents are exemplified by those already mentioned above. Additional excipients such as sweetening, flavouring and colouring agents, may also be present.

The pharmaceutical compositions of the invention may also be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, an esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavouring and preservative agents.

Syrups and elixirs may be formulated with sweetening agents such as glycerol, propylene glycol, sorbitol, aspartame or sucrose, and may also contain a demulcent, preservative, flavouring and/or colouring agent.

The pharmaceutical compositions may also be in the form of a sterile injectable aqueous or oily suspension, which may be formulated according to known procedures using one or more of the appropriate dispersing or wetting agents and suspending agents, which have been mentioned above. A sterile injectable preparation may also be a sterile injectable solution or suspension in a non-toxic parenterally-acceptable diluent or solvent, for example a solution in 1,3-butanediol.

Compositions for administration by inhalation may be in the form of a conventional pressurised aerosol arranged to dispense the active ingredient either as an aerosol containing finely divided solid or liquid droplets. Conventional aerosol propellants such as volatile fluorinated hydrocarbons or hydrocarbons may be used and the aerosol device is conveniently arranged to dispense a metered quantity of active ingredient.

For further information on formulation the reader is referred to Chapter 25.2 in Volume 5 of Comprehensive Medicinal Chemistry (Corwin Hansch; Chairman of Editorial Board), Pergamon Press 1990.

The amount of active ingredient that is combined with one or more excipients to produce a single dosage form will necessarily vary depending upon the host treated and the particular route of administration. For example, a formulation intended for oral administration to humans will generally contain, for example, from 0.5 mg to 2 g of active agent compounded with an appropriate and convenient amount of excipients which may vary from about 5 to about 98 percent by weight of the total composition. Dosage unit forms will generally contain about 1 mg to about 500 mg of an active ingredient. For further information on Routes of Administration and Dosage Regimes the reader is referred to Chapter 25.3 in Volume 5 of Comprehensive Medicinal Chemistry (Corwin Hansch; Chairman of Editorial Board), Pergamon Press 1990.

As stated above the size of the dose required for the therapeutic or prophylactic treatment of a particular disease state will necessarily be varied depending on the host treated, the route of administration and the severity of the illness being treated. In one aspect of the invention a daily dose in the range of 1-50 mg/kg is employed. However the daily dose will necessarily be varied depending upon the host treated, the particular route of administration, and the severity of the illness being treated. Accordingly the optimum dosage may be determined by the practitioner who is treating any particular patient.

In addition to its use in therapeutic medicine, compounds of formula (I) and their pharmaceutically acceptable salts are also useful as pharmacological tools in the development and standardisation of in-vitro and in-vivo test systems for the evaluation of the effects of inhibitors of DNA gyrase and/or topoisomerase IV in laboratory animals such as cats, dogs, rabbits, monkeys, rats and mice, as part of the search for new therapeutic agents.

In the above other, pharmaceutical composition, process, method, use and medicament manufacture features, the alternative and particular embodiments of the compounds of the invention described herein also apply.

### Combinations

The compounds of the invention described herein may be applied as a sole therapy or may involve, in addition to a compound of the invention, one or more other substances and/or treatments. Such conjoint treatment may be achieved by way of the simultaneous, sequential or separate administration of the individual components of the treatment. Where the administration is sequential or separate, the delay in administering the second component should not be such as to lose the beneficial effect of the combination. Suitable classes and substances may be selected from one or more of the following:

i) other antibacterial agents for example macrolides e.g. erythromycin, azithromycin or clarithromycin; quinolones e.g. ciprofloxacin or levofloxacin; β-lactams e.g. penicillins e.g. amoxicillin or piperacillin; cephalosporins e.g. ceftriaxone or ceftazidime; carbapenems, e.g. meropenem or imipenem etc; aminoglycosides e.g. gentamicin or tobramycin; or oxazolidinones; and/or
ii) anti-infective agents for example, an antifungal triazole e.g. or amphotericin; and/or
iii) biological protein therapeutics for example antibodies, cytokines, bactericidal/permeability-increasing protein (BPI) products; and/or
iv) one or more antibacterial agents useful in the treatment of Mycobacterium tuberculosis such as one or more of rifampicin, isoniazid, pyrizinamide, ethambutol, quinolones e.g. moxifloxacin or gatifloxacin, streptomycin.
v) efflux pump inhibitors.
Therefore, in a further aspect of the invention there is provided a compound of the formula (I), or a pharmaceutically acceptable salt thereof and a chemotherapeutic agent selected from:
   i) one or more additional antibacterial agents; and/or
   ii) one or more anti-infective agents; and/or
   iii) biological protein therapeutics for example antibodies, cytokines, bactericidal/permeability-increasing protein (BPI) products; and/or
   iv) one or more antibacterial agents useful in the treatment of pulmonary tuberculosis, extra-pulmonary tuberculosis, avium infections, buruli ulcers and/or
   v) one or more efflux pump inhibitors.

### EXAMPLES

The invention is now illustrated but not limited by the following Examples in which unless otherwise stated:—

(i) Evaporations were carried out by rotary evaporation in-vacuo and work-up procedures were carried out after removal of residual solids by filtration;
(ii) Operations were generally carried out at ambient temperature, that is typically in the range 18-26° C. and without exclusion of air unless otherwise stated, or unless the skilled person would otherwise work under an inert atmosphere;
(iii) Column chromatography (by the flash procedure) was used to purify compounds and was performed on Merck Kieselgel silica (Art. 9385) unless otherwise stated;
(iv) Yields are given for illustration only and are not necessarily the maximum attainable;
(v) The structure of the end-products of the invention were generally confirmed by NMR and mass spectral techniques; proton magnetic resonance spectra is quoted and was generally determined in DMSO-d₆ unless otherwise stated using a Bruker DRX-300 spectrometer operating at a field strength of 300 MHz. Chemical shifts are reported in parts per million downfield from tetramethysilane as an internal standard (δ scale) and peak multiplicities are shown thus: s, singlet; d, doublet; AB or dd, doublet of doublets; dt, doublet of triplets; dm, doublet of multiplets; t, triplet, m, multiplet; br, broad;
(vi) Fast-atom bombardment (FAB) mass spectral data were generally obtained using a Platform spectrometer (supplied by Micromass) run in electrospray and, where appropriate, either positive ion data or negative ion data were collected or using Agilent 1100series LC/MSD equipped with Sedex 75ELSD, run in atmospheric pressure chemical ionisation mode and, where appropriate, either positive ion data or negative ion data were collected; mass spectra were run with an electron energy of 70 electron volts in the chemical ionization (CI) mode using a direct exposure probe; where indicated ionization was effected by electron impact (EI), fast atom bombardment (FAB) or electrospray (ES); values for m/z are given; generally, only ions which indicate the parent mass are reported;
(vii) each intermediate was generally purified to the standard required for the subsequent stage and was characterised in sufficient detail to confirm that the assigned structure was correct; purity was assessed by high pressure liquid chromatography, thin layer chromatography, or NMR and identity was determined by infra-red spectroscopy (IR), mass spectroscopy or NMR spectroscopy as appropriate;
(vii) the following abbreviations may be used:

DMF is N,N-dimethylformamide;

SM is starting material;

DMSO is dimethylsulfoxide;

CDCl₃ is deuterated chloroform;

MS is mass spectroscopy;

EtOAc is ethyl acetate;

THF is tetrahydrofuran;

MeOH is methanol;

TFA is trifluoroacetic acid;

EtOH is ethanol;

DCM is dichloromethane; and

(viii) temperatures are quoted as ° C.
(ix) RT is room temperature
(x) NA is not available

| | | Eco-tolC_{—} | Spn548_{—} | | Mtu |
|---|---|---|---|---|---|
| Example | | MIC | Mean MIC | Msm_GyrB_{—} | MIC |
| No. | Structure | (μg/ml) | (μg/ml) | IC50 (μM) | (μg/ml) |
| 1 | 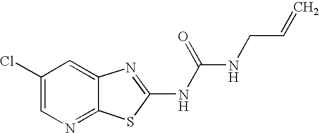 | 16 | 8 | 0.1907 | 16 |
| 2 | 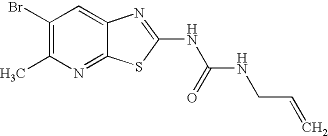 | 32 | >32 | 2.187 | |
| 3 | 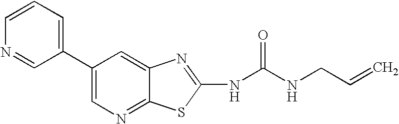 | >64 | 1 | 0.08809 | 10.08 |
| 4 | 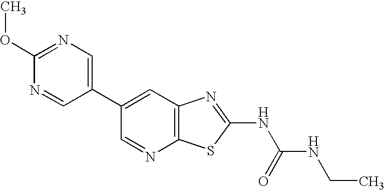 | >64 | 0.5 | 0.1401 | >8 |
| 5 | 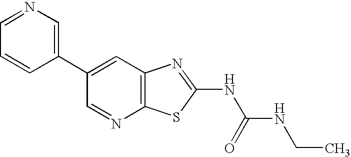 | 8.83 | 1.56 | | |
| 6 | 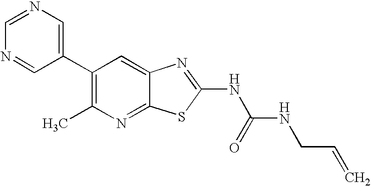 | 32.32 | 1.01 | 0.1507 | 8 |
| 7 | 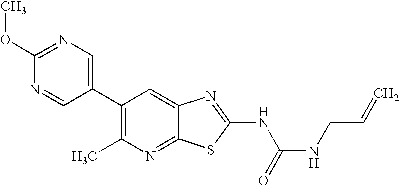 | >32 | 0.0625 | 0.2392 | 32 |
| 8 | 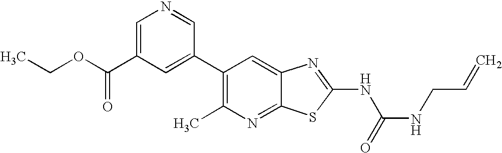 | >39.75 | >39.75 | 0.1505 | >32 |
| 9 | 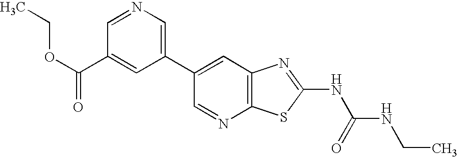 | >37.14 | 0.5803 | 0.0511 | >32 |
| 10 | 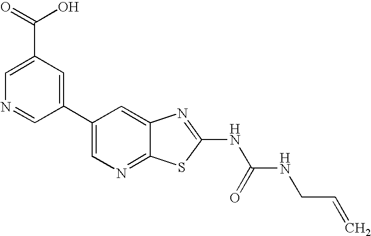 | >64 | 32 | 0.1853 | >16 |
| 11 | 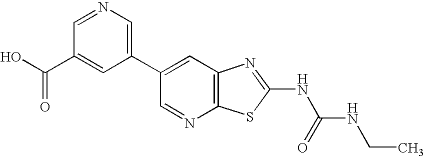 | >34.34 | 17.17 | 0.1585 | 32 |
| 12 | 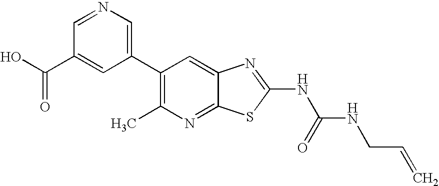 | >32 | 32 | 0.1655 | >32 |
| 13 | 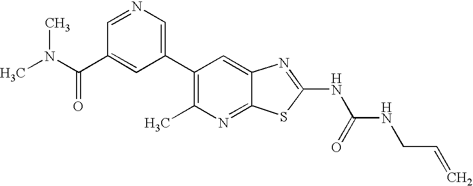 | >32 | 4 | 0.6439 | >32 |
| 14 | 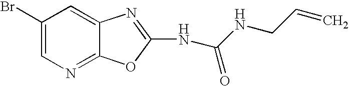 | 32 | 16 | 24.67 | >16 |
| 15 | 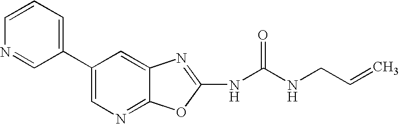 | >64 | 16 | 2.005 | >16 |
| 16 | 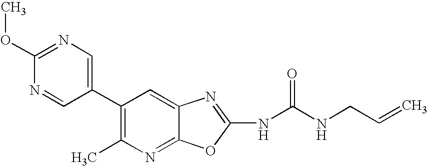 | >32 | 16 | 1.61 | >32 |
| 17 | 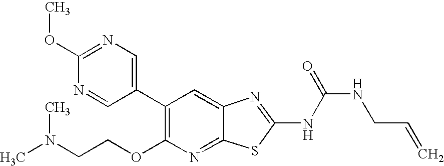 | >64 | 16 | 0.216 | >64 |
| 18 | 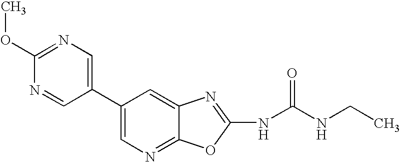 | >32 | >32 | 10.89 | >32 |
| 19 | 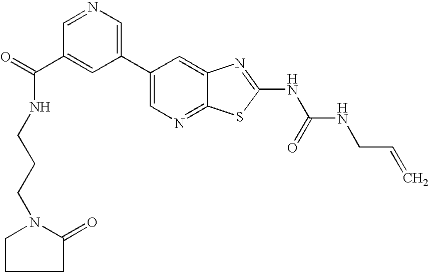 | >32 | >32 | 0.2973 | 32 |
| 20 | 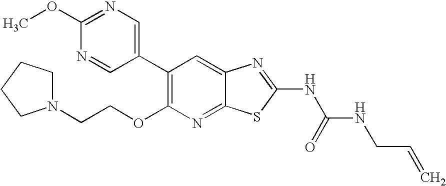 | >32 | >32 | 0.2589 | >32 |
| 21 | 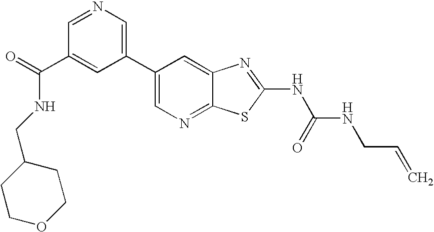 | >32 | >32 | 0.2359 | >64 |
| 22 | 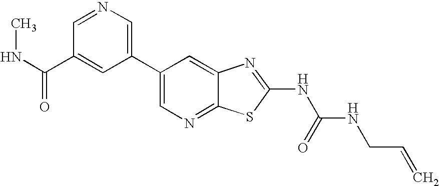 | >32 | >32 | 0.3234 | >64 |
| 23 | 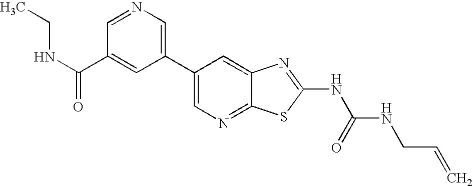 | >32 | >32 | 0.4043 | >64 |
| 24 | 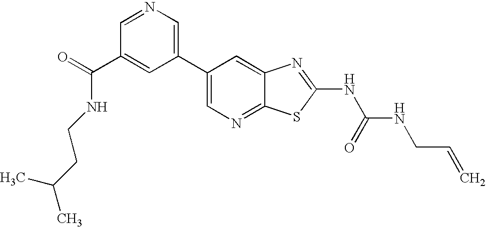 | >32 | >32 | 0.5834 | >64 |
| 25 | 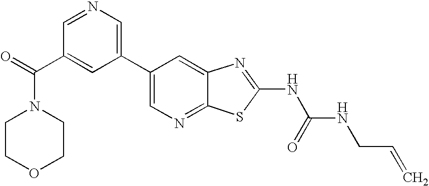 | >32 | >32 | 1.442 | >32 |
| 26 | 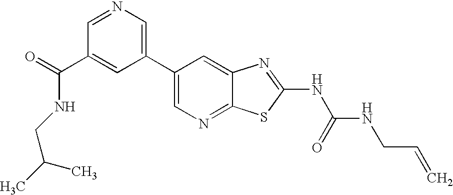 | >32 | >32 | 0.3945 | >32 |
| 27 | 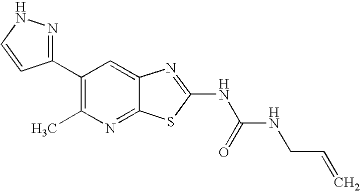 | >22.63 | 2 | 1.836 | 11.31 |
| 28 | 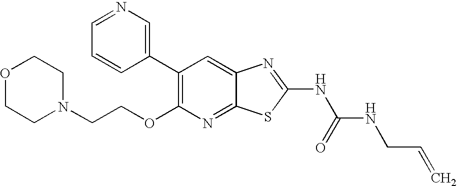 | >32 | 4 | 0.4006 | >22.63 |
| 29 | 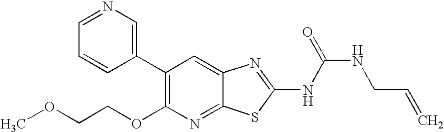 | 1.219 | 0.1523 | 0.04655 | 4 |
| 30 | 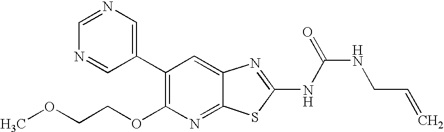 | 0.6094 | 0.1523 | 0.06032 | 2 |
| 31 | 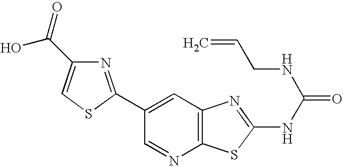 | >32 | >32 | 8.944 | >32 |
| 32 | 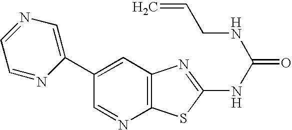 | >32 | 32 | 0.8355 | >32 |
| 33 | 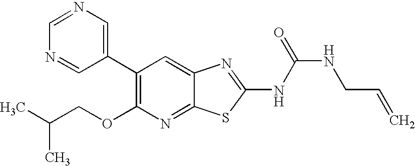 | 1 | 0.0625 | 0.01839 | 1 |
| 34 | 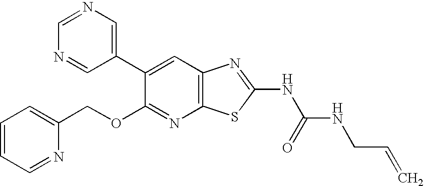 | 1 | 0.0625 | 0.02807 | 1.414 |
| 35 | 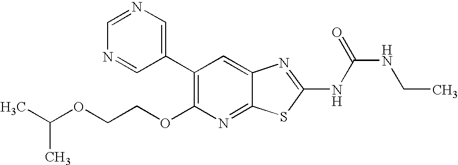 | 0.25 | 0.0625 | 0.028 | 0.3536 |
| 36 | 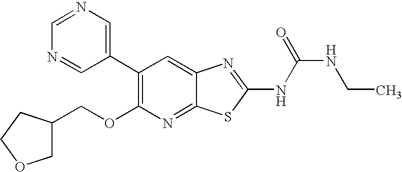 | 0.125 | 0.0625 | 0.009892 | 0.05441 |
| 37 | 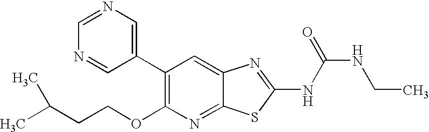 | 0.25 | 0.0625 | 0.01176 | <0.25 |
| 38 | 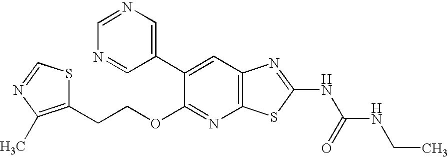 | 0.125 | 0.0625 | <0.005012 | 0.2806 |
| 39 | 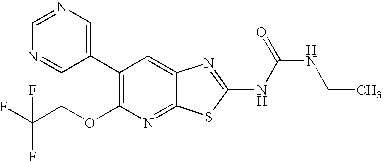 | 0.3536 | 0.0625 | <0.003867 | 0.25 |
| 40 | 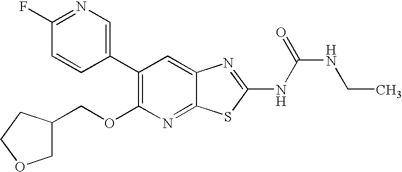 | 0.5711 | 0.07139 | <0.00254 | 0.3536 |
| 41 | 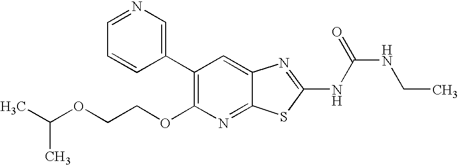 | 5.019 | 0.07842 | 0.009102 | 2 |
| 42 | 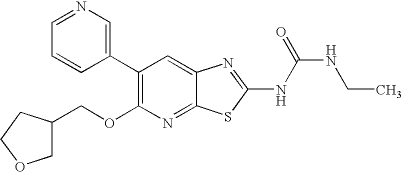 | 1.248 | 0.07802 | 0.004825 | 0.4204 |
| 43 | 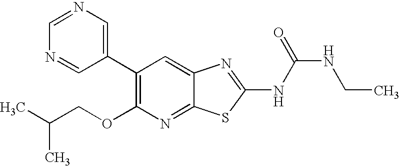 | 0.582 | 0.07274 | 0.004394 | 0.125 |
| 44 | 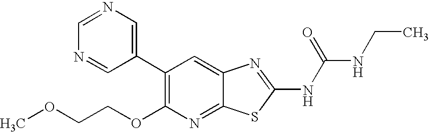 | 1.17 | 0.07313 | 0.01751 | 0.25 |
| 45 | 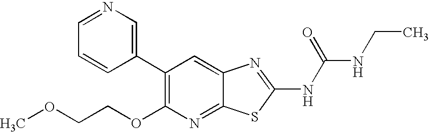 | 4.668 | 0.2917 | 0.02524 | 1 |
| 46 | 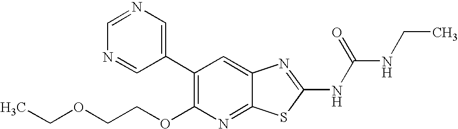 | 0.25 | 0.0625 | 0.02 | 0.125 |
| 47 | 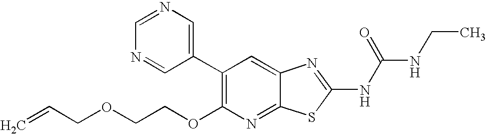 | 0.5 | 0.0625 | 0.01715 | 0.125 |
| 48 | 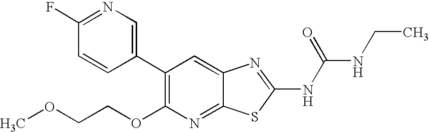 | 0.125 | 0.0625 | 0.004514 | 0.7071 |
| 49 | 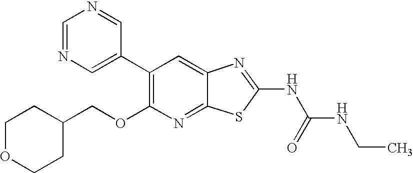 | 0.125 | 0.0625 | <0.00254 | 0.0625 |
| 50 | 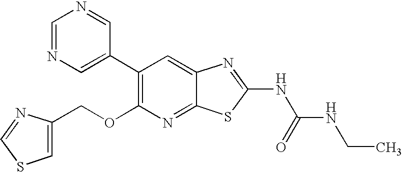 | 0.25 | 0.0625 | <0.00254 | 0.25 |
| 51 | 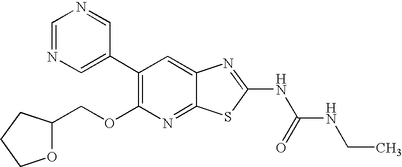 | 0.125 | 0.0625 | 0.004866 | 0.25 |
| 52 | 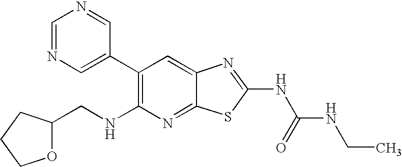 | 4 | 0.125 | 0.007878 | 1 |
| 53 | 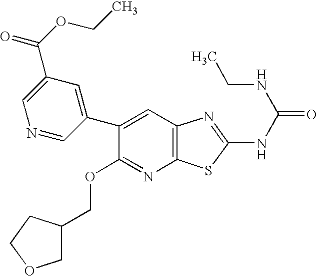 | 0.5 | 0.0625 | 0.06221 | 2.828 |
| 54 | 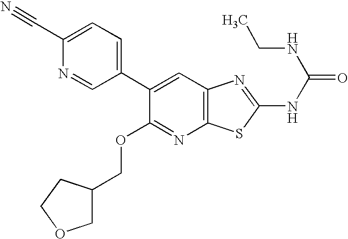 | 1 | 0.0625 | 0.01417 | 0.1768 |
| 55 | 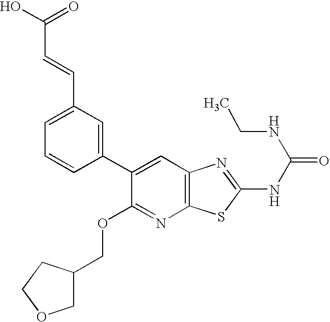 | 2 | 0.0625 | 0.03523 | >8 |
| 56 | 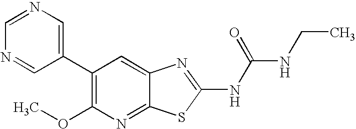 | 0.5 | 0.0625 | 0.02157 | 0.7071 |
| 57 | 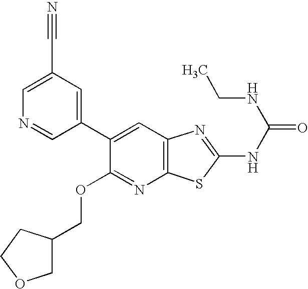 | 0.0625 | 0.0625 | 0.01244 | 0.25 |
| 58 | 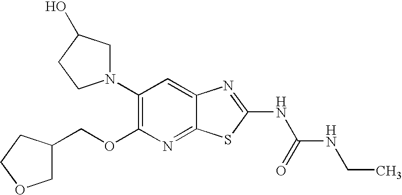 | >32 | 4 | 2.11 | >8 |
| 59 | 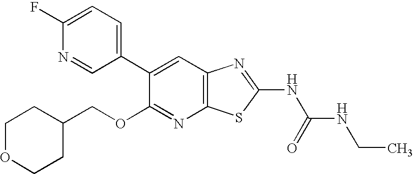 | 1 | 0.125 | <0.004833 | 0.5 |
| 60 | 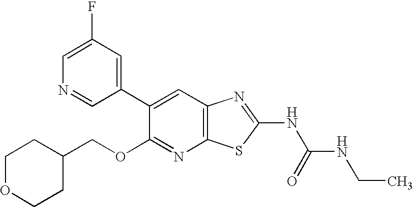 | 1 | 0.125 | <0.00254 | 0.3536 |
| 61 | 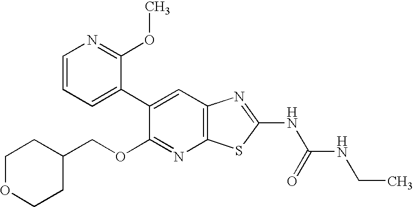 | 16 | 0.25 | 0.004739 | >4 |
| 62 | 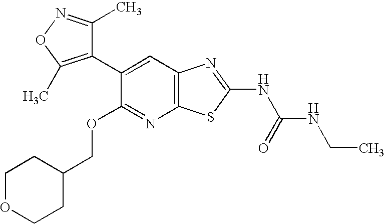 | 64 | 8 | 0.7269 | >4 |
| 63 | 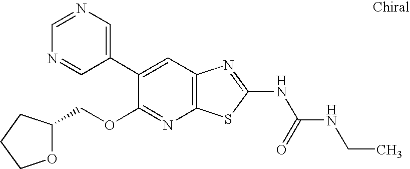 | 1 | 0.125 | 0.0026 | 0.25 |
| 64 | 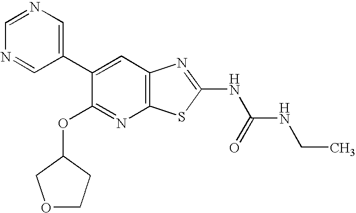 | 0.5 | 0.125 | <0.00254 | 0.125 |
| 65 | 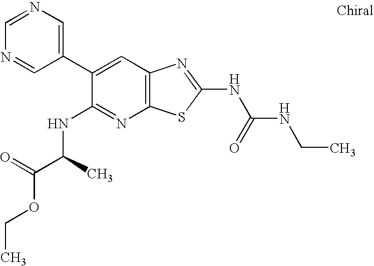 | 4 | 0.125 | <0.00254 | 0.125 |
| 66 | 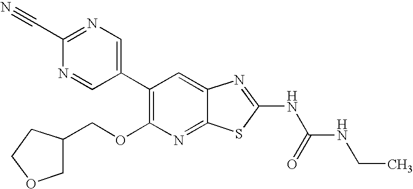 | 0.5 | 0.125 | <0.00254 | <0.04301 |
| 67 | 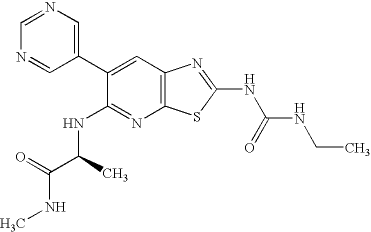 | >64 | 2 | 0.01587 | 1.414 |
| 68* | 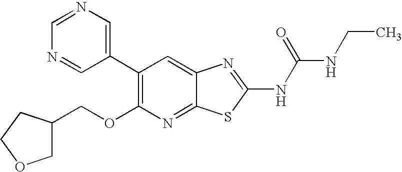 | 0.5 | 0.125 | 0.007756 | 0.08839 |
| 69* | 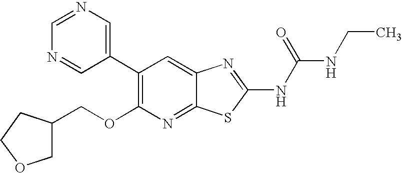 | 0.25 | 0.125 | 0.007508 | 0.08839 |
| 70 | 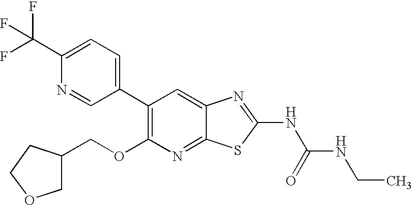 | 0.25 | 0.0625 | 0.006917 | 4.757 |
| 71 | 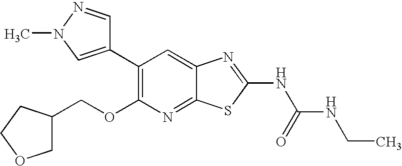 | 0.5 | 0.125 | 0.02495 | 0.7071 |
| 72 | 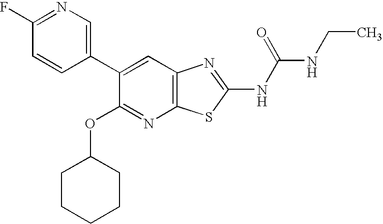 | 0.25 | 0.0625 | 0.02915 | 2 |
| 73 | 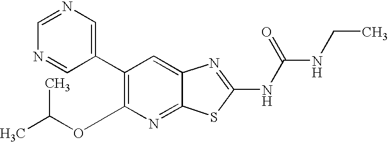 | NA | NA | 0.005205 | 0.125 |
| 74 | 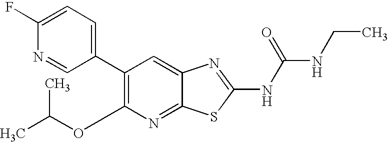 | NA | NA | 0.01001 | 1 |
| 75 | 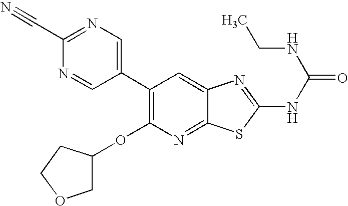 | NA | NA | <0.00254 | <0.06 |
| 76 | 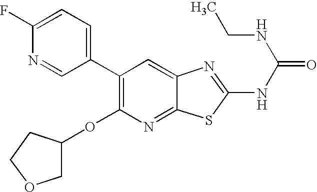 | NA | NA | 0.003536 | 0.5 |
| 77 | 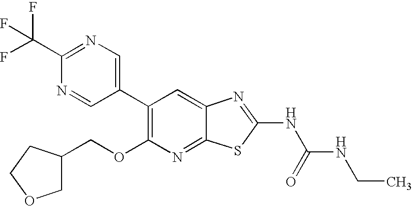 | NA | NA | 0.0029 | 2.828 |
| 78 | 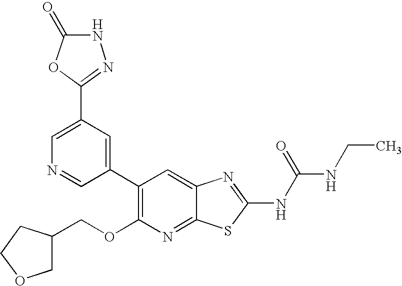 | NA | NA | <0.00254 | 8 |
| 79 | 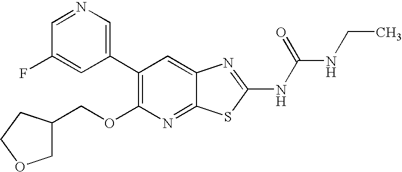 | NA | NA | <0.00254 | 0.3536 |

* Example 68 and 69 are optically pure enantiomers obtained by chiral resolution of Example 43 on chiral HPLC. Absolute stereochemistry is yet unknown.
NA—data not available

### Synthesis of Compounds

Experimental Section: Reactions were carried out in anhydrous solvents under an atmosphere of Nitrogen unless otherwise stated and monitored by thin layer chromatography using Merck F254 silica gel plates. LC-MS was performed on an Agilent 1100 equipped with a C18 RRHT analytical column (1.8μ, 4.6 mm×50 mm), Photo Diode Array detector and a single quadrupole mass spectrometer (electro spray ionization). ¹H NMR was recorded on a Bruker Avance 300 spectrometer in (CD₃)₂SO or CDCl₃ with tetramethylsilane as an internal standard. Reagents were purchased from commercial suppliers such as Sigma-Aldrich, Fluka, ABCR, Across, Lancaster, Maybridge, and other commercial vendors.

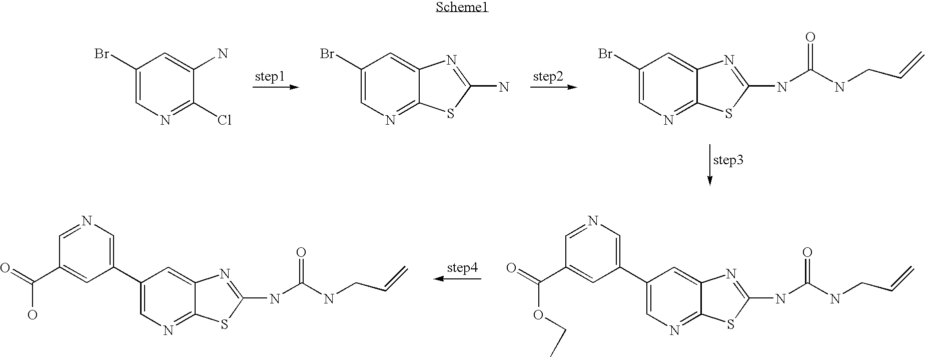

### Step 1. 6-Bromo-thiazolo[5,4-b]pyridin-2-yl amine: (Intermediate 1)

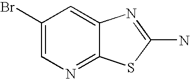

In a 50 ml RB flask, 5-bromo-2-chloropyridin-3-amine (3.11 g, 15 mmol) was taken in conc. HCl (30 mL) and sonicated well to give pale brown solution. To this potassium thiocyanate (2.187 g, 22.50 mmol) was added and the resulting mixture was heated at 100° C. for 6 hrs. The reaction mixture was changed to pale yellow suspension after 30 minutes of reflux. The reaction mixture was evaporated in vacuo; ice-cold water was added to the residue, sonicated well and neutralized with saturated sodium carbonate under cooling condition. The precipitated solid was sonicated well, filtered and dried under high vacuum afforded the product as off-white solid (2.5 gm)

MS (ES⁺): 231 for C₆H₄BrN₃S

¹H NMR δ(DMSO-d₆): 5.85 (bs, 2H, NH2); 7.3 (s, 1H, Aro); 7.65 (s, 1H, Aro).

The following Intermediates 2-3 were prepared in a manner analogous to step 1

| Intermediates | Compound | M/Z | SM |
|---|---|---|---|
| Intermediate 2 | 6-Bromo-5-methyl-thiazolo | 245 | 5-Bromo-2-chloro- |
| | [5,4-b] pyridin-2-ylamine | | 6-methyl-pyridin-3- |
| | | | ylamine |
| | | | (Intermediate4) |
| Intermediate 3 | 6-Chloro-thiazolo [5,4-b] | 186 | 2-Bromo-5-chloro- |
| | pyridin-2-ylamine | | pyridin-3-ylamine |
| | | | (Commercial) |

### Intermediate 4: 5-Bromo-2-chloro-6-methyl-pyridin-3-ylamine

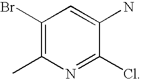

In a 250 ml RB flask, 3-bromo-6-chloro-2-methyl-5-nitropyridine (1.5 g, 5.97 mmol, commerical) was dissolved in ethyl acetate (20 mL). To this solution, ammonium chloride (3.19 g, 59.65 mmol) dissolved in water (10 ml) was added and stirred at RT for 10 minutes. Then zinc powder (2.340 g, 35.79 mmol) was added at once and the resulting reaction mixture was refluxed at 55° C. for 6 hrs. The reaction mixture was filtered through celite and concentrated in vacuo. The residue was partitioned between ethyl acetate (150 ml) and water (75). The organic layer was dried over anhydrous sodium sulphate and concentrated in vacuo. The crude product was purified by Flash column chromatography using Argonaut purification system, which was eluted with 12% ethyl acetate in hexane to give 5-bromo-2-chloro-6-methylpyridin-3-amine (0.500 g, 37.8%) as white solid.

MS (ES⁺): 222 for C₆H₆BrClN₂

### Step 2: 1-allyl-3-(6-bromothiazolo[5,4-b]pyridin-2-yl) urea (Intermediate 5)

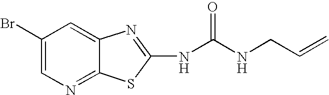

In a 25 ml round-bottomed flask, 6-bromothiazolo[5,4-b]pyridin-2-amine (0.575 g, 2.5 mmol) was suspended in tetrahydrofuran (15 mL). To this triethylamine (0.697 mL, 5.00 mmol) was added in one portion and resulting reaction mixture was stirred at RT. Then allyl isocyanate (0.331 mL, 3.75 mmol) was added and stirred at RT for overnight. The reaction mixture was evaporated in vacuo, ice-cold water was added, sonicated well and the precipitated solid was filtered and dried under high vacuum. The crude product was triturated with acetonitrile gave the pure product as brown solid (0.650 mg, 83%).

MS (ES⁺): 314 for C₁₀H₉BrN₄OS

¹H NMR (DMSO-d6) δ: 3.82 (t, 2H, CH2); 5.10-5.25 (m, 2H, CH2); 5.80-5.95 (m, 1H, CH); 6.95 (t, 1H, NH); 8.15 (bs, 1H, NH); 8.23 (s, 1H, Aro.); 8.48 (s, 1H, Aro.).

The following compounds were prepared in a manner analogous to step 2 (intermediate 5) starting from corresponding amines and isocyanates (commercially available).

| Intermediates | Compound | ¹H NMR (DMSO-d6) δ | M/Z | SM |
|---|---|---|---|---|
| Intermediate 6 | 1-(6-Bromo- | | 302 | 6-Bromo- |
| | thiazolo [5,4-b] | | | thiazolo [5,4- |
| | pyridin-2-yl)-3- | | | b] pyridin-2-yl |
| | ethyl-urea | | | amine |
| | | | | (Intermediate |
| | | | | 1) |
| Example1 | 1-Allyl-3-(6- | 3.85 (t, 2H, CH2); | 269 | 6-Chloro- |
| | chloro-thiazolo | 5.10-5.25 (m, 2H, CH2); | | thiazolo [5,4- |
| | [5,4-b] pyridin- | 5.85-5.97 (m, 1H, CH); 6.95 (t, | | b] pyridin-2- |
| | 2-yl)-urea | 1H, NH); 8.11 (s, | | ylamine |
| | | 1H, Aro); 8.41 (s, | | (Intermediate |
| | | 1H, Aro.); 11.1 (bs, 1H, | | 3) |
| | | NH) | | |
| Example 2 | 1-Allyl-3-(6- | 2.65 (s, 3H, CH3) 3.90 (t, | 328 | 6-Bromo-5- |
| | bromo-5- | 2H, CH2); 5.10-5.25 (m, | | methyl- |
| | methyl-thiazolo | 2H, CH2); 5.85-5.97 (m, | | thiazolo [5,4- |
| | [5,4-b] pyridin- | 1H, CH); 6.95 (t, | | b] pyridin-2- |
| | 2-yl)-urea | 1H, NH); 8.20 (s, | | ylamine |
| | | 1H, Aro); 11.1 (bs, 1H, | | (Intermediate |
| | | NH) | | 2) |

### Step 3: 5-[2-(3-Allyl-ureido)-thiazolo[5,4-b]pyridin-6-yl]-nicotinic acid ethyl ester (Intermediate 7)

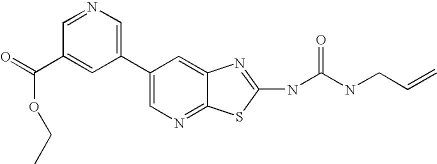

In a 25 ml microwave vial, 1-allyl-3-(5-bromobenzo[d] thiazol-2-yl)urea (300 mg, 0.96 mmol, intermediate 5) in ethylene glycol dimethyl ether (5 mL) was taken, ethyl 5-(4,4,5,5-tetramethyl-1,3,2-dioxaborolan-2-yl)nicotinate (346 mg, 1.25 mmol), tetrakis (triphenylphosphine) palladium (0) (111 mg, 0.10 mmol) and sodium bicarbonate (1M, 1.92 mmol) were added. The resulting mixture was subjected to microwave irradiation at 140° C. for 5 minutes. The TLC showed absence of starting material, the reaction mixture was concentrated under reduced pressure. The crude product was purified by column chromatography using silica gel (60-120) mesh eluted with (0-2%) methanol:chloroform. The fractions having product were concentrated under reduced pressure afforded the title compound as pale yellow colored solid (160 mg, 43.5%).

MS (ES⁺): 383 for C₁₉H₁₈N₄O₃S

The following compounds were synthesized by an analogous method to Intermediate 7 using corresponding commercially available boronic acids.

| Example | Compound Name | M/Z | ¹HNMR (300 MHz)(δ) | SM |
|---|---|---|---|---|
| 3 | 1-allyl-3-[6-(3-pyridyl)thiazolo | 312 | H¹NMR [DMSO-d⁶]: | Intermediate 5 |
| | [5,4-b] pyridin- | | 3.85 (t, 2H, CH2); | |
| | 2-yl] urea | | 5.13-5.30 (m, 2H, CH2); | |
| | | | 5.85-5.90 (m, 1H, CH); 6.97 (t, | |
| | | | 1H, NH); 7.50-7.60 (m, | |
| | | | 1H, Aro.); 8.20 (m, 1H, | |
| | | | Aro.); 8.33 (s, 1H, Aro.); | |
| | | | 8.65 (m, 1H, Aro.); 8.75 (s, | |
| | | | 1H, Aro.); 9.0 (s, | |
| | | | 1H, Aro.); 11.0 (bs, | |
| | | | 1H, NH); | |
| 4 | 1-ethyl-3-[6-(2- | 328 | H¹NMR [DMSO-d⁶]: | Intermediate 6 |
| | methoxypyrimidin-5-yl)thiazolo | | 1.12 (t, 3H, CH3); 3.20 (m, | |
| | [5,4-b] pyridin- | | 2H, CH2); 4.0 (s, | |
| | 2-yl] urea | | 3H, OCH3); 6.98 (t, | |
| | | | 1H, NH); 8.30 (s, 1H, | |
| | | | Aro.); 8.70 (s, 1H, Aro.); | |
| | | | 9.05 (s, 1H, Aro.); | |
| | | | 11.15 (bs, 1H, NH); | |
| 5 | 1-Ethyl-3-(6-pyridin-3- | 300 | H¹NMR [DMSO-d⁶]: | Intermediate 6 |
| | yl-thiazolo [5, | | 1.09 (t, 3H, CH3); 3.20 (m, | |
| | 4-b] pyridin-2-yl)- | | 2H, CH2); 6.77 (t, | |
| | urea | | 1H, NH); 7.34-7.67 (m, | |
| | | | 1H, Aro); 8.21 (d, 1H, | |
| | | | Aro.); 8.30 (d, 1H, Aro.); | |
| | | | 8.62 (d, 1H, Aro.); 8.71 (d, | |
| | | | 1H, Aro.); 9.0 (s, 1H, Aro.); | |
| | | | 110.97 (s, 1H, NH); | |
| 6 | 1-Allyl-3-(5-methyl-6- | 327 | H¹NMR [DMSO-d⁶]: | Example 2 |
| | pyrimidin-5-yl-thiazolo | | 2.55 (s, 3H, CH3); 3.95 (t, | |
| | [5,4-b] pyridin-2-yl)- | | 2H, CH2); 5.10-5.25 (m, | |
| | urea | | 2H, CH2); 5.82-6.0 (m, | |
| | | | 1H, CH); 6.97 (t, 1H, NH); | |
| | | | 7.97 (s, 1H, Aro.); 8.98 (s, | |
| | | | 1H, Aro.); 9.30 (s, Aro.); | |
| | | | 10.95 (bs, 1H, NH) | |
| 7 | 1-Allyl-3-[6-(2- | 357 | H¹NMR [DMSO-d⁶]: | Example 2 |
| | methoxy-pyrimidin-5- | | 2.55 (s, 3H, CH3); 3.87 (t, | |
| | yl)-5-methyl-thiazolo | | 2H, CH2); 4.0 (s, | |
| | [5,4-b] pyridin-2-yl]- | | 3H, OCH3); 5.10-5.25 (m, | |
| | urea | | 2H, CH2); 5.82-6.0 (m, | |
| | | | 1H, CH); 6.97 (t, 1H, NH); | |
| | | | 7.97 (s, 1H, Aro.); 8.75 (s, | |
| | | | 1H, Aro.); 10.90 (bs, | |
| | | | 1H, NH). | |
| 8 | 5-[2-(3-Allyl-ureido)-5- | 398 | H¹NMR [DMSO-d⁶]: | Example 2 |
| | methyl-thiazolo [5,4-b] | | 1.35 (t, 3H, CH3); 2.52 (s, | |
| | pyridin-6-yl]-nicotinic | | 3H, CH3); 3.87 (t, | |
| | acid ethyl ester | | 2H, CH2); 4.37-4.45 (m, | |
| | | | 2H, OCH2); 5.10-5.25 (m, | |
| | | | 2H, CH2); 5.82-6.0 (m, | |
| | | | 1H, CH); 6.95 (t, 1H, NH); | |
| | | | 7.92 (s, 1H, Aro.); 8.31 (s, | |
| | | | 1H, Aro.); 8.95 (s, 1H, | |
| | | | Aro.); 9.15 (s, 1H, Aro.); | |
| | | | 10.95 (bs, 1H, NH). | |
| 9 | 5-[2-(3-Ethyl-ureido)- | 372 | H¹NMR [DMSO-d⁶]: | Intermediate 6 |
| | thiazolo [5,4-b] pyridin- | | 1.11 (t, 3H, CH3); 1.37 (t, | |
| | 6-yl]-nicotinic acid | | 3H, CH3); 3.20 (m, | |
| | ethyl ester | | 2H, CH2); 4.40 (m, | |
| | | | 2H, OCH2); 7.0 (t, | |
| | | | 1H, NH); 8.30 (s, 1H, | |
| | | | Aro.); 8.60 (s, 1H, Aro.); | |
| | | | 8.75 (s, 1H, Aro.); 9.1 (s, | |
| | | | 1H, Aro.); 9.2 (s, 1H, Aro.); | |
| | | | 11.25 (bs, 1H, NH); | |
| 27 | 1-[5-methyl-6-(1H- | 315.1 | 2.70 (s, 3H), 3.82 (t, 2H), | Example 2 |
| | pyrazol-3- | | 5.10 (d, 1H), 5.20 (d, 1H), | |
| | yl)[1,3]thiazolo[5,4- | | 5.85-5.95 (m, 1H), 6.65 (s, | |
| | b]pyridin-2-yl]-3-prop- | | 1H), 6.90 (t, 1H), 7.80 (s, | |
| | 2-en-1-ylurea | | 1H), 8.01 (s, 1H), | |
| | | | 10.80 (b, 1H), 13.00 (b, 1H) | |
| 31 | 2-{2-[(prop-2-en-1- | 362.1 | 3.85 (t, 2H), 5.11 (d, 1H), | Intermediate 2 |
| | ylcarbamoyl)amino][1, | | 5.20 (d, 1H), | |
| | 3]thiazolo[5,4- | | 5.85-5.95 (m, 1H), 6.95 (b, 1H), | |
| | b]pyridin-6-yl}-1,3- | | 8.43 (s, 1H), 8.60 (s, 1H), | |
| | thiazole-4-carboxylic | | 8.97 (s, 1H), 11.25 (b, | |
| | acid | | 1H), 13.25 (b, 1H) | |
| 32 | 1-prop-2-en-1-yl-3-(6- | 313.1 | 3.85 (t, 2H), 5.11 (d, 1H), | Intermediate 5 |
| | pyrazin-2- | | 5.20 (d, 1H), | |
| | yl[1,3]thiazolo[5,4- | | 5.85-5.94 (m, 1H), 6.95 (b, 1H), | |
| | b]pyridin-2-yl)urea | | 8.67 (s, 1H), 8.70 (s, 1H), | |
| | | | 8.80 (s, 1H), 9.14 (s, 1H), | |
| | | | 9.45 (s, 1H), 11.15 (b, 1H) | |

### Example 10

### Step 4: 5-[2-(3-Allyl-ureido)-thiazolo[5,4-b]pyridin-6-yl]-nicotinic acid

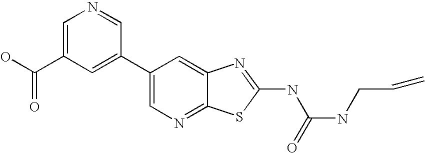

A solution of sodium hydroxide (5M, 2.87 mmol) in water (1 ml) was added to a stirred solution of ethyl 5-(2-(3-allylureido) thiazolo[5,4-b]pyridin-6-yl)nicotinate (55 mg, 0.14 mmol) in MeOH (5 mL) and the resultant solution was stirred overnight. The reaction mixture was concentrated and dissolved in water (5 ml). The solution was acidified with 1N hydrochloric acid (pH4-5) and the precipitate that formed was collected, washed with water and air dried (30 mg, 58.9%).

MS (ES⁺): 356 for C₁₆H₁₃N₅O₃S

H¹NMR [DMSO-d⁶]: 3.85 (t, 2H, CH2); 5.10-5.30 (m, 2H, CH2); 5.85-5.97 (m, 1H, CH); 7.55 (s, 1H, Aro.); 7.75 (t, 1H, NH); 8.30 (s, 1H, Aro.); 8.60 (s, 1H, Aro.); 8.80 (s, 1H, Aro.); 9.15 (s, 1H, Aro.); 11.50 (bs, 1H, NH).

The following compounds were synthesized by an analogous method to Example 10.

| Example | Compound Name | M/Z | ¹HNMR (300 MHz)(δ) | SM |
|---|---|---|---|---|
| 11 | 5-[2-(3-Ethyl-ureido)- | 344 | H¹NMR [D₂O]: | Example 9 |
| | thiazolo [5,4-b] | | 0.97 (t, 3H, CH3); 2.95 (m, 2H, CH2); | |
| | pyridin-6-yl]-nicotinic | | 7.50 (s, 1H, Aro); 8.0 (s, 1H, Aro.); | |
| | acid | | 8.20 (s, 1H, Aro.); 8.55 (s, | |
| | | | 1H, Aro.); 8.65 (s, 1H, Aro.). | |
| 12 | 5-[2-(3-Allyl-ureido)- | 370 | H¹NMR [DMSO-d⁶]: 2.45 (s, | Example 8 |
| | 5-methyl thiazolo | | 3H, CH3); 3.85 (t, 2H, CH2); 5.3 (m, | |
| | [5,4b] pyridin-6-yl]nicotinic | | 2H, CH2); 5.95 (m, 1H, CH); 6.95 (t, | |
| | acid | | 1H, NH); 7.90 (s, 1H, Aro); 8.3 (s, | |
| | | | 1H, Aro); 8.9 (d, 1H, Aro.); 9.15 (d, | |
| | | | 1H, Aro.); 10.95 (bs, 1H, NH); | |
| | | | 13.5 (bs, 1H, COOH); | |

### Example 13

### 5-[2-(3-Allyl-ureido)-5-methyl-thiazolo[5,4-b]pyridin-6-yl]-N,N-dimethyl-nicotinamide

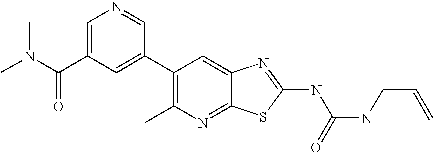

Dimethylamine 40% aqueous solution (2.15 ml, 0.14 mmol) and ethyl 5-(2-(3-allylureido) thiazolo[5,4-b]pyridin-6-yl)nicotinate (55 mg, 0.14 mmol) were taken together and stirred at RT for 2 hrs. The reaction mixture was concentrated; ice-cold water was added and extracted with dichloromethane (3×15 ml). The combined organic layer was dried over anhydrous sodium sulphate and evaporated in vacuo afforded the title compound as white solid (25 mg, 40%).

MS (ES⁺): 397 for C₁₉H₂₀N₆O₂S

H¹NMR [DMSO-d⁶]: 2.45 (s, 3H, CH3); 3.05 (d, 6H, 2CH3); 3.85 (t, 2H, CH2); 5.3 (m, 2H, CH2); 5.95 (m, 1H, CH); 6.9 (t, 1H, NH); 7.85 (s, 1H, Aro); 7.95 (t, 1H, Aro); 8.65 (d, 1H, Aro.); 8.75 (d, 1H, Aro.); 10.95 (bs, 1H, NH);

The following compounds were synthesized by an analogous method to Example 13 using Example 10 as an intermediate

| | | M/Z | |
|---|---|---|---|
| Example | Compound Name | (M + 1) | ¹HNMR (300 MHz) (d) |
| 19 | N-[3-(2-oxopyrrolidin-1-yl)propyl]- | 480.2 | 1.78 (qn, 2H), 1.92 (qn, 2H), |
| | 5-{2-[(prop-2-en-1-ylcarbamoyl)amino][1, | | 2.25 (t, 2H), 3.38 (t, 2H), |
| | 3]thiazolo[5,4-b]pyridin-6- | | 3.20-3.32 (m, 4H), 3.85 (t, 2H), |
| | yl}pyridine-3-carboxamide | | 5.13 (d, 1H), 5.20 (d, 1H), |
| | | | 5.85-5.95 (m, 1H), 6.91 (t, 1H), 8.40 (s, |
| | | | 1H), 8.55 (s, 1H), 8.70 (t, 1H), |
| | | | 8.80 (d, 1H), 9.0 (s, 1H), 9.12 (s, |
| | | | 1H), 11.00 (bs, 1H) |
| 21 | 5-{2-[(prop-2-en-1-ylcarbamoyl)amino][1, | 453.2 | 1.15-1.20 (m, 2H), 1.60-1.70 (m, |
| | 3]thiazolo[5,4-b]pyridin-6- | | 2H), 1.78-1.92 (m, 1H), |
| | yl}-N-(tetrahydro-2H-pyran-4- | | 3.20-3.35 (m, 4H), 3.80-3.95 (m, 4H), |
| | ylmethyl)pyridine-3-carboxamide | | 5.10 (d, 1H), 5.20 (d, 1H), |
| | | | 5.82-5.98 (m, 1H), 6.90 (t, 1H), |
| | | | 8.40 (s, 1H), 8.55 (s, 1H), 8.72 (t, |
| | | | 1H), 8.80 (s, 1H), 9.00 (s, 1H), |
| | | | 9.11 (s, 1H), 11.00 (bs, 1H) |
| 22 | N-methyl-5-{2-[(prop-2-en-1- | 369.1 | 2.85 (d, 3H), 3.85 (t, 2H), |
| | ylcarbamoyl)amino][1,3]thiazolo[5,4- | | 5.12 (d, 1H), 5.20 (d, 1H), |
| | b]pyridin-6-yl}pyridine-3- | | 5.85-5.98 (m, 1H), 6.90 (t, 1H), 8.40 (s, |
| | carboxamide | | 1H), 8.55 (s, 1H), 8.65-8.75 (m, |
| | | | 1H), 8.80 (s, 1H), 9.00 (s, 1H), |
| | | | 9.15 (s, 1H), 11.00 (b, 1H) |
| 23 | N-ethyl-5-{2-[(prop-2-en-1- | 383.1 | 1.20 (t, 3H), 3.30-3.40 (m, 2H), |
| | ylcarbamoyl)amino][1,3]thiazolo | | 3.85 (t, 2H), 5.12 (d, 1H), |
| | [5,4-b]pyridin-6-yl}pyridine-3- | | 5.20 (d, 1H), 5.85-5.95 (m, 1H), |
| | carboxamide | | 6.90 (t, 1H), 8.40 (s, 1H), 8.50 (s, |
| | | | 1H), 8.71 (t, 1H), 8.80 (s, 1H), |
| | | | 9.00 (s, 1H), 9.12 (s, 1H), |
| | | | 11.00 (b, 1H) |
| 24 | N-(3-methylbutyl)-5-{2-[(prop-2-en- | 425.2 | 0.95 (d, 6H), 1.48 (q, 2H), |
| | 1-ylcarbamoyl)amino][1, | | 1.60-1.72 (m, 1H), 3.30-3.40 (m, 2H), |
| | 3]thiazolo[5,4-b]pyridin-6- | | 3.85 (t, 2H), 5.15 (d, 1H), |
| | yl}pyridine-3-carboxamide | | 5.21 (d, 1H), 6-85-6.98 (m, 1H), |
| | | | 6.92 (t, 1H), 8.40 (s, 1H), 8.55 (s, |
| | | | 1H), 8.65 (t, 1H), 8.80 (s, 1H), |
| | | | 9.00 (s, 1H), 9.11 (s, 1H), |
| | | | 11.00 (b, 1H) |
| 25 | 1-{6-[5-(morpholin-4- | 425.1 | 3.30-3.80 (m, 8H), 3.85 (t, 2H), |
| | ylcarbonyl)pyridin-3-yl][1,3]thiazolo[5, | | 5.11 (d, 1H), 5.20 (d, 1H), |
| | 4-b]pyridin-2-yl}-3-prop- | | 5.82-5.95 (m, 1H), 6.90 (t, 1H), 8.25 (s, |
| | 2-en-1-ylurea | | 1H), 8.38 (s, 1H), 8.65 (s, 1H), |
| | | | 8.77 (s, 1H), 9.10 (s, 1H), |
| | | | 11.00 (b, 1H) |
| 26 | N-(2-methylpropyl)-5-{2-[(prop-2- | 411.1 | 0.95 (d, 6H), 1.82-1.98 (m, 1H), |
| | en-1-ylcarbamoyl)amino][1, | | 3.15 (t, 2H), 3.85 (t, 2H), 5.13 (d, |
| | 3]thiazolo[5,4-b]pyridin-6- | | 1H), 5.21 (d, 1H), 5.85-6.00 (m, |
| | yl}pyridine-3-carboxamide | | 1H), 6.90 (t, 1H), 8.40 (s, 1H), |
| | | | 8.55 (s, 1H), 8.70 (t, 1H), 8.80 (s, |
| | | | 1H), 9.12 (s, 1H), 9.15 (s, 1H), |
| | | | 11.00 (b, 1H) |

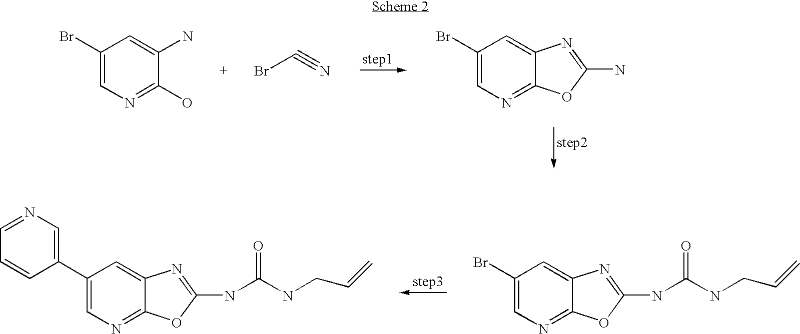

### Step 1: 6-bromooxazolo[5,4-b]pyridin-2-amine (Intermediate 8)

In a 25 ml RB flask, was taken cyanogen bromide (0.254 mg) in water (5.00 mL). To this 3-amino-5-bromopyridin-2-ol (0.378 g, 2 mmol) in ethanol (5 mL) was added and the resulting mixture was heated at 100° C. for 15 minutes. The reaction mixture was evaporated in vacuo; ice-cold water was added to the residue, sonicated well and neutralized with saturated sodium bicarbonate under cooling condition. The precipitated solid was sonicated well, filtered and dried under high vacuum. The solid was taken in methanol and DCM (1:1, 50 ml)) mixture sonicated well and filtered. The filtrate was concentrated in vacuo and the residue was triturated with diethyl ether and filtered afforded the title compound as yellow solid (300 mg, 70.1%).

MS (ES⁺): 215 for C₆H₄BrN₃O

¹H NMR δ(DMSO-d₆): 7.65 (s, 1H, Aro); 7.91 (s, 1H, Aro). 7.97 (bs, 2H, NH2);

### Intermediate 9: 6-Bromo-5-methyl-oxazolo[5,4-b]pyridin-2-ylamine

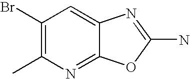

Intermediate 9 was synthesized by an analogous method to intermediate 8 starting from 3-amino-5-bromo-6-methyl-pyridin-2-ol (Commercial source, Princeton)

MS (ES⁺): 229 for C₇H₆BrN₃O

### Example 14

### Step 2: 1-allyl-3-(6-bromooxazolo[5,4-b]pyridin-2-yl)urea was synthesized by an analogous method to intermediate 5 starting from intermediate 8

MS (ES⁺): 298 for C₁₀H₉BrN₄O₂

¹H NMR δ(DMSO-d6): 3.82 (m, 2H, CH2); 5.05-5.20 (m, 2H, CH2); 5.80-5.95 (m, 1H, CH); 7.87 (s, 1H, Aro.); 7.95 (s, 1H, Aro.).

### Intermediate 10: 1-Allyl-3-(6-bromo-5-methyl-oxazolo[5,4-b]pyridin-2-yl)- urea

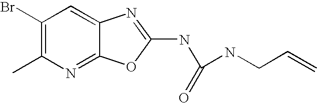

Intermediate 10 was synthesized by an analogous method to intermediate 5 starting from intermediate 9

MS (ES⁺): 312 for C₁₁H₁₁BrN₄O₂

### Step 3: 1-allyl-3-(6-(pyridin-3-yl)oxazolo[5,4-b]pyridin-2-yl)urea (Example 15) was synthesized by an analogous method to intermediate 7 using Example 14 as an intermediate

MS (ES⁺): 296

¹H NMR δ(DMSO-d6): 3.85 (t, 2H, CH2); 5.13-5.30 (m, 2H, CH2); 5.85-5.90 (m, 1H, CH); 7.50 (m, 1H, Aro.); 7.80 (m, 2H, Aro.); 8.20 (m, 1H, Aro.); 8.35 (bs, 1H, NH.); 8.60 (s, 1H, Aro.); 9.0 (s, 1H, Aro.); 11.0 (bs, 1H, NH);

Following examples were synthesized by an analogous method to Example 15

| | | MS | | |
|---|---|---|---|---|
| Example | Compound Name | (ES⁺): | ¹HNMR (DMSO-d6) δ | SM |
| 16 | 3-allyl-1-[6-(2- | 341.1 | 2.45 (s, 3H), 3.92 (t, | Intermediate |
| | methoxypyrimidin-5-yl)- | | 2H), 4.00 (s, 3H), | 10 |
| | 5-methyl-oxazolo[5,4- | | 5.12 (d, 1H), 5.24 (d, 1H), | |
| | b]pyridin-2-yl]urea | | 5.85-5.98 (m, 1H), | |
| | | | 7.88 (s, 1H), 8.31 (t, 1H), | |
| | | | 8.71 (s, 2H), 11.21 (b, | |
| | | | 1H) | |
| 18 | 1-ethyl-3-[6-(2- | 315.1 | 1.15 (t, 3H), 3.30 (qn, | 1-ethyl-3-(6- |
| | methoxypyrimidin-5- | | 2H), 4.00 (s, 3H), | bromooxazolo |
| | yl)[1,3]oxazolo[5,4- | | 8.18 (t, 1H), 8.28 (d, 1H), | [5,4-b] |
| | b]pyridin-2-yl]urea | | 8.44 (d, 1H), 9.00 (s, | pyridin-2-yl)urea |
| | | | 2H), 10.50 (b, 1H) | |

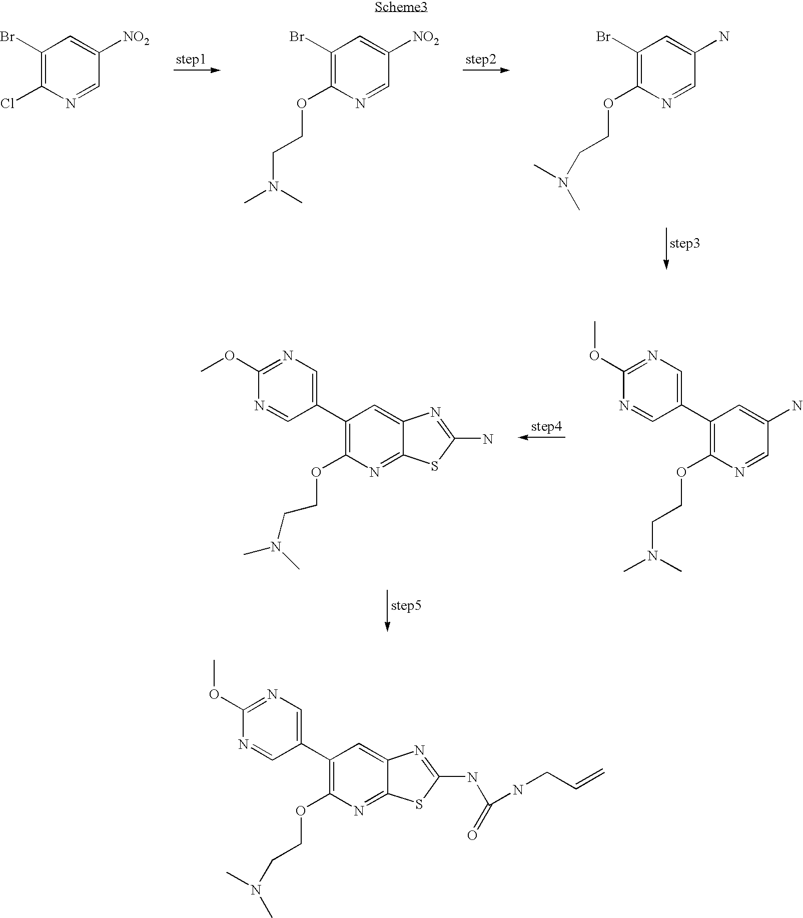

### Step 1: 2-(3-bromo-5-nitropyridin-2-yloxy)-N,N-dimethylethanamine (Intermediate 11)

To a stirred solution of 3-bromo-2-chloro-5-nitropyridine (2.5 g, 10.53 mmol) and 2-(dimethylamino)ethanol (1.877 g, 21.06 mmol) in DMF (10 mL) was added portion wise potassium carbonate (2.91 g, 21.06 mmol) and the mixture was stirred at 60° C. for 2-3 hrs. Reaction mixture was cooled to RT, diluted with ethyl acetate (50-70 ml), washed with water and then brine, organic layer was collected, dried over sodium sulfate and concentrated in vacuo to give crude 2-(3-bromo-5-nitropyridin-2-yloxy)-N,N-dimethylethanamine (2.70 g, 88%) as brown liquid. The crude material was taken for next step without further purification.

MS (ES⁺): 292 for C₉H₁₂BrN₃O₃

### Step 2: 5-Bromo-6-(2-dimethylamino-ethoxy)-pyridin-3-ylamine (Intermediate 12)

2-(3-bromo-5-nitropyridin-2-yloxy)-N,N-dimethylethanamine (1 g, 3.45 mmol, Intermediate 11) was dissolved in ethyl acetate (20 mL), to this solution zinc (1.352 g, 20.68 mmol) powder was added followed by addition of aq solution of ammonium chloride (1.844 g, 34.47 mmol). This suspension was stirred at 25° C. for ½ hrs. The reaction mixture was filtered through celite.filtrate was collected and diluted with water (50 ml), The aq layer was back extracted with ethyl Acetate (3×100 mL). Combined the organic layer was washed with brine solution (1×50 mL), dried Na2SO4, filtered and concentrated in vacuo to give the 5-bromo-6-(2-(dimethylamino)ethoxy)pyridin-3-amine (0.800 g, 89%).

MS (ES⁺): 262 for C₉H₁₄BrN₃O

### Step 3: 6-(2-Dimethylamino-ethoxy)-5-(2-methoxy-pyrimidin-5-yl)-pyridin-3-ylamine (Intermediate 13)

To a stirred solution of 5-bromo-6-(2-(dimethylamino)ethoxy)pyridin-3-amine (800 mg, 3.08 mmol, Intermediate 12) in dimethoxy ethane (20 mL), 2-methoxypyrimidin-5-ylboronic acid (710 mg, 4.61 mmol) was added to the reaction mixture, nitrogen was purged for 5-10 minutes to remove dissolved oxygen. To this PalladiumTetrakis (533 mg, 0.46 mmol) was added followed by addition of aq solution of sodium carbonate (652 mg, 6.15 mmol). The resulting reaction mixture was heated at 91° C. for 4 hrs. Solvent from the reaction mixture was evaporated in vacuo and the crude product was purified by fish chromatography using 8% MeOH/DCM as solvent system to give 6-(2-(dimethylamino)ethoxy)-5-(2-methoxypyrimidin-5-yl)pyridin-3-amine (400 mg, 45.0%).

MS (ES⁺): 290 for C₁₄H₉N₅O₂

### Step 4: 5-(2-Dimethylamino-ethoxy)-6-(2-methoxy-pyrimidin-5-yl)-thiazolo[5,4-b]pyridin-2-ylamine (Intermediate 14)

To a solution of 6-(2-(dimethylamino)ethoxy)-5-(2-methoxypyrimidin-5-yl)pyridin-3-amine (350 mg, 1.21 mmol, Intermediate 13) in acetic acid (5 mL) were added sodium acetate (794 mg, 9.68 mmol) and potassium thiocyanate (705 mg, 7.26 mmol) and stirred at 5-10° C. Then BROMINE (0.093 mL, 1.81 mmol) was added with cooling with ice-water bath. Then the reaction mixture was stirred at 25° C. for 1 hr. Reaction mixture was diluted with ethyl acetate (100 ml), washed with water and aq odium sulfite solution. The organic layer was collected, dried over sodium sulfate and conc in vacuo. Aq layer was neutralised with sodium carbonate pH(8) and then extracted with 25% MeOH/DCM solution. The organic layer was collected, dried over sodium sulfate and conc to give 5-(2-(dimethylamino)ethoxy)-6-(2-methoxypyrimidin-5-yl)thiazolo[5,4-b]pyridin-2-amine (250 mg, 59.7%) which is used for next without further purification.

MS (ES⁺): 347 for C₁₅H₁₈N₆O₂S

### Example 17

### Step 5: 1-allyl-3-(5-(2-(dimethylamino)ethoxy)-6-(2-methoxypyrimidin-5-yl) thiazolo[5,4-b]pyridin-2-yl)urea

To a stirred solution of intermediate 14 (150 mg, 0.43 mmol) in tetrahydrofuran (1 mL), triethylamine (0.121 mL, 0.87 mmol) was added followed by addition of allyisocyanate (180 mg, 2.17 mmol). Reaction mix was stirred at 100° C. for 48 hrs. Solvent is from the reaction mixture was evaporated in vauo. The crude product was purified by flash chromatography using 8% MeOH/DCM as solvent system to give 1-allyl-3-(5-(2-(dimethylamino)ethoxy)-6-(2-methoxypyrimidin-5-yl)thiazolo[5,4-b]pyridin-2-yl)urea (55.0 mg, 29.6%) off white solid.

MS (ES⁺): 430 for C₁₉H₂₃N₇O₃S

H¹NMR [DMSO-d⁶]: 2.20 (s, 6H, 2CH3); 2.65 (t, 2H, CH2); 3.90 (t, 2H, CH2); 4.05 (s, 3H, OCH3); 4.45 (t, 3H, CH2); 5.10-5.30 (m, 2H, CH2); 5.85-6.0 (m, 1H, CH); 6.95 (t, 1H, NH); 8.10 (s, 1H, Aro); 8.90 (s, 2H, Aro); 11.80 (bs, 1H, NH).

Following examples were made by similar procedures described under scheme 3 for Example 17.

| | | MS | |
|---|---|---|---|
| Example | Compound | (ES⁺): | ¹HNMR (DMSO-d6) δ |
| 20 | 1-[6-(2-methoxypyrimidin- | 456.2 | 1.60-1.70 (m, 4H), 2.40-2.50 (m, |
| | 5-yl)-5-(2-pyrrolidin-1- | | 4H), 2.80 (t, 2H), 3.81 (t, 2H), |
| | ylethoxy)[1,3]thiazolo[5,4- | | 4.00 (s, 3H), 4.45-4.50 (m, 2H), |
| | b]pyridin-2-yl]-3-prop-2-en- | | 5.11 (d, 1H), 5.20 (d, 1H), |
| | 1-ylurea | | 5.81-5.95 (m, 1H), 6.81 (t, 1H), 8.10 (s, |
| | | | 1H), 8.90 (s, 2H), 10.70 (bs, 1H) |
| 28 | 1-[5-(2-morpholin-4- | ES⁻ = 439.1 | 1.30-1.50 (m, 6H), 2.35-2.45 (m, |
| | ylethoxy)-6-pyridin-3- | | 4H), 2.65 (t, 2H), 3.80 (t, 2H), |
| | yl[1,3]thiazolo[5,4- | | 5.10 (d, 1H), 5.20 (d, 1H), |
| | b]pyridin-2-yl]-3-prop-2-en- | | 5.82-5-98 (m, 1H), 6.88 (t, 1H), |
| | 1-ylurea | | 7.41-7.49 (m, 1H), 8.04 (s, 1H), |
| | | | 8.05-8.11 (m, 1H), 8.55 (d, 1H), 8.88 (s, |
| | | | 1H), 10.75 (b, 1H) |

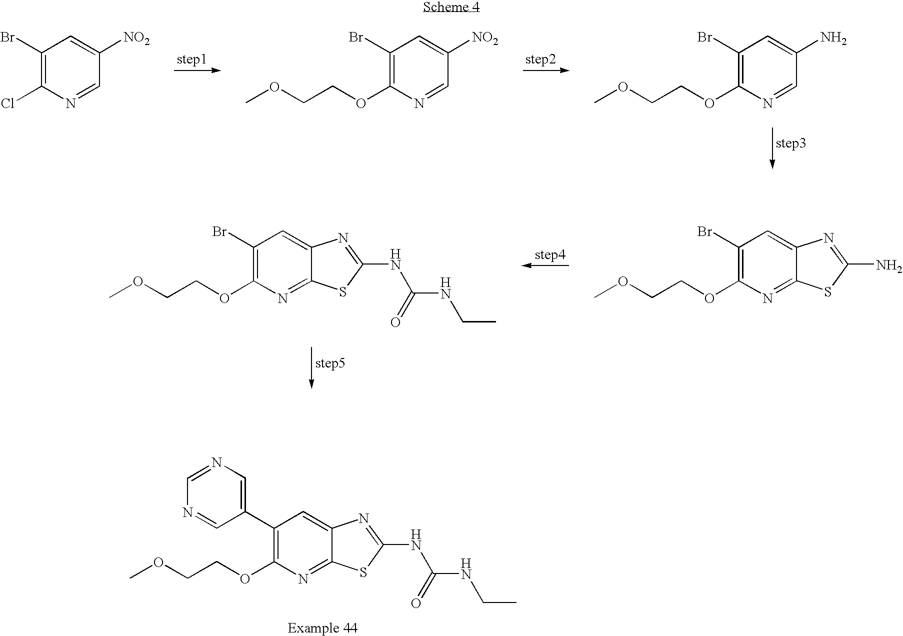

### Step 1: 3-bromo-2-(2-methoxyethoxy)-5-nitropyridine (Intermediate 15)

To a stirred solution of 3-bromo-2-chloro-5-nitropyridine (1.5 g, 6.32 mmol) and 2-methoxyethanol (0.961 g, 12.63 mmol) in DMF (10 mL), potassium carbonate (1.746 g, 12.63 mmol) was added portion wise and the mixture was stirred at 60° C. for 5 hr. Reaction mixture was then cooled to RT, diluted with ethyl acetate (150 ml), washed successively with water and then brine, organic layer was collected, dried over sodium sulfate and concentrated to give crude 3-bromo-2-(2-methoxyethoxy)-5-nitropyridine (1.500 g, 86%) as brown solid.

MS (ES⁺): 277.9, for C₈H₉BrN₂O₄

### Step 2: 5-bromo-6-(2-methoxyethoxy)pyridin-3-amine (Intermediate 16)

3-bromo-2-(2-methoxyethoxy)-5-nitropyridine (1.5 g, 5.41 mmol) was dissolved in ethyl acetate (100 mL), to this solution zinc (2.478 g, 37.90 mmol) powder was added followed by addition of slurry of ammonium chloride (2.90 g, 54.14 mmol) in water (10 mL). This suspension was stirred at RT for 6 hr. Reaction mixture was filtered through Celite. Celite bed was thoroughly washed with ethyl acetate. Filtrate was combined, washed with brine, dried on sodium sulfate and concentrated to give crude 5-bromo-6-(2-methoxyethoxy)pyridin-3-amine (1.20 g, 90%).

MS (ES⁺): 247.8 for C₈H₁₁BrN₂O₂

### Step 3: 6-bromo-5-(2-methoxyethoxy)thiazolo[5,4-b]pyridin-2-amine (Intermediate 17)

Potassium thiocyanate (3.78 g, 38.85 mmol) was added to a solution of 5-bromo-6-(2-methoxyethoxy)pyridin-3-amine (1.2 g, 4.86 mmol) in acetic acid (15 mL). The mixture was cooled in ice-water bath and bromine (0.375 mL, 7.28 mmol) was added drop-wise to it. The reaction mixture was then stirred at RT for 1 hr. Reaction mixture was then heated at 110° C. for 10-20 min. Hot mixture was filtered through sintered funnel, solid was washed with acetic acid (15 mL) and water (20 mL). Filtrate was basified to pH 8 with sodium carbonate and extracted with 15%MeOH/DCM mixture. Organic layers were combined, dried on sodium sulfate and concentrated under vacuum. Solid residue was triturated with small volume of methanol, filtered and dried to afford 6-bromo-5-(2-methoxyethoxy)thiazolo[5,4-b]pyridin-2-amine (1.000 g, 67.7%) as brownish yellow coloured solid

MS (ES⁺): 305.0 for C₉H₁₀BrN₃O₂S

### Step 4: 1-(6-bromo-5-(2-methoxyethoxy)thiazolo[5,4-b]pyridin-2-yl)-3-ethylurea (Intermediate 18)

In a 20 mL capacity microwave vial 6-bromo-5-(2-methoxyethoxy)thiazolo[5,4-b]pyridin-2-amine (500 mg, 1.64 mmol) was suspended in mixture of tetrahydrofuran (3 mL) and toluene (3 mL) was taken. Added triethylamine (0.458 mL, 3.29 mmol) to this mixture followed by addition of ethyl isocyanate (467 mg, 6.58 mmol). Added catalytic amount of dibutyltin oxide (5 mg, 0.02 mmol). Reaction mixture was microwaved at 110° C. for 45 min. Reaction mixture was then concentrated under vacuum. Solid residue was triturated with small volume of methanol (5 mL), filtered and dried to afford pure 1-(6-bromo-5-(2-methoxyethoxy)thiazolo[5,4-b]pyridin-2-yl)-3-ethylurea (435 mg, 70.5%) as off-white solid.

MS (ES⁺): 376.0 for C₁₂H₁₅BrN₄O₃S

### Step 5: 1-ethyl-3-[5-(2-methoxyethoxy)-6-pyrimidin-5-yl[1,3]thiazolo[5,4-b]pyridin-2-yl]urea (Example 44)

In a microwave vial 1-(6-bromo-5-(2-methoxyethoxy)thiazolo[5,4-b]pyridin-2-yl)-3-ethylurea (125 mg, 0.33 mmol), pyrimidin-5-ylboronic acid (83 mg, 0.67 mmol) and sodium bicarbonate (56.0 mg, 0.67 mmol) were mixed in DME (8 mL) and water (2 mL). The mixture was purged with N₂ for 5-10 min. Added Pd(PPh₃)₄ (57.7 mg, 0.05 mmol) to the mixture and it was microwaved for 1 hr 20 min at 115° C. Reaction mixture was concentrated under vacuum. Added water (10 ml) to the residue and extracted thrice with dichloromethane. Organic layers were combined, dried over sodium sulphate and concentrated under vacuum. Residue was purified by flash chromatography on silica gel column using 5% MeOH/DCM as eluent. Pure fractions were combined and evaporated under vacuum to afford pure 1-ethyl-3-(5-(2-methoxyethoxy)-6-(pyrimidin-5-yl)thiazolo[5,4-b]pyridin-2-yl)urea (65.0 mg, 52.1%) as white crystalline material.

MS (ES⁺): 375.1 for C₁₆H₁₈N₆O₃S

¹H NMR (DMSOD₆) δ: 1.10 (t, 3H), 3.20 (qn, 2H), 3.30 (s, 3H), 3.68 (t, 2H), 4.50 (t, 2H), 6.68 (t, 1H), 8.20 (s, 1H), 9.10 (s, 2H), 9.18 (s, 1H), 10.75 (b, 1H)

Following examples were made by using similar protocol described in Scheme 4 for Example 44.

| | Molecule | | |
|---|---|---|---|
| Example | IUPAC Name (ACD) | Mass | 1HNMR (300 MHz)(δ) |
| 29 | 1-[5-(2-methoxyethoxy)-6-pyridin- | 386.1 | 3.25 (s, 3H), 3.70 (t, 2H), |
| | 3-yl[1,3]thiazolo[5,4-b]pyridin-2- | | 3.85 (t, 2H), 4.50 (t, 2H), 5.15 (d, 1H), |
| | yl]-3-prop-2-en-1-ylurea | | 5.20 (d, 1H), 5.85-6.00 (m, 1H), |
| | | | 8.85 (t, 1H), 7.45-7.50 (m, 1H), |
| | | | 8.10 (s, 2H), 8.60 (d, 1H), 8.22 (s, |
| | | | 1H), 8.85 (s, 1H), 10.75 (b, 1H) |
| 30 | 1-[5-(2-methoxyethoxy)-6- | 387.1 | 3.27 (s, 3H), 3.70 (t, 2H), 3.85 (t, |
| | pyrimidin-5-yl[1,3]thiazolo[5,4- | | 2H), 4.50 (t, 2H), 5.15 (d, 1H), |
| | b]pyridin-2-yl]-3-prop-2-en-1-ylurea | | 5.20 (d, 1H), 5.85-5.95 (m, 1H), |
| | | | 6.85 (m, 1H), 8.22 (s, 1H), 9.10 (s, |
| | | | 2H), 9.17 (s, 1H), 10.80 (b, 1H) |
| 33 | 1-[5-(2-methylpropoxy)-6- | 385.1 | 0.95 (d, 6H), 2.05 (m, 1H), 3.80 (t, |
| | pyrimidin-5-yl[1,3]thiazolo[5,4- | | 2H), 4.15 (d, 2H), 5.11 (d, 1H), |
| | b]pyridin-2-yl]-3-prop-2-en-1-ylurea | | 5.21 (d, 1H), 5.85-5.95 (m, 1H), |
| | | | 6.85 (t, 1H), 8.19 (s, 1H), 9.18 (s, |
| | | | 2H), 9.20 (s, 1H), 10.70 (b, 1H). |
| 34 | 1-prop-2-en-1-yl-3-[5-(pyridin-2- | 420.1 | 3.81 (t, 2H), 5.10 (d, 1H), 5.20 (d, |
| | ylmethoxy)-6-pyrimidin-5- | | 1H), 5.55 (s, 2H), 5.82-5.98 (m |
| | yl[1,3]thiazolo[5,4-b]pyridin-2- | | 1H), 6.88 (t, 1H), 7.30-7.35 (m, |
| | yl]urea | | 1H), 7.42 (d, 1H), 7.80 (t, 1H), |
| | | | 8.20 (s, 1H), 8.55 (d, 1H), 9.14 (s, |
| | | | 2H), 9.17 (s, 1H), 10.80 (b, 1H) |
| 35 | 1-ethyl-3-{5-[2-(1- | 403.2 | 1.05 (d, 6H), 1.10 (t, 3H), 3.20 (qn, |
| | methylethoxy)ethoxy]-6-pyrimidin- | | 2H), 3.55-3.65 (m, 1H), 3.70 (t, |
| | 5-yl[1,3]thiazolo[5,4-b]pyridin-2- | | 2H), 4.48 (t, 2H), 6.68 (t, 1H), |
| | yl}urea | | 8.20 (s, 1H), 9.12 (s, 2H), 9.15 (s, |
| | | | 1H), 10.68 (b, 1H) |
| 36 | 1-ethyl-3-[6-pyrimidin-5-yl-5- | 401.1 | 1.10 (t, 3H), 1.60-1.70 (m, 1H), |
| | (tetrahydrofuran-3- | | 1.90-2.02 (m, 1H), 2.58-2.70 (m, |
| | ylmethoxy)[1,3]thiazolo[5,4- | | 1H), 3.19 (qn, 2H), 3.55 (dd, 1H), |
| | b]pyridin-2-yl]urea | | 3.65 (qn, 1H), 3.70 (q, 2H), |
| | | | 4.25-4.40 (m, 2H), 6.70 (t, 1H), 8.18 (s, |
| | | | 1H), 9.06 (s, 2H), 9.17 (s, 1H), |
| | | | 10.60 (b, 1H) |
| 37 | 1-ethyl-3-[5-(3-methylbutoxy)-6- | 387.2 | 0.90 (d, 6H), 1.10 (t, 3H), 1.63 (qn, |
| | pyrimidin-5-yl[1,3]thiazolo[5,4- | | 2H), 1.65-1.75 (m, 1H), 3.25 (qn, |
| | b]pyridin-2-yl]urea | | 2H), 4.40 (t, 2H), 6.70 (t, 1H), |
| | | | 8.15 (s, 1H), 9.05 (s, 2H), 9.15 (s, 1H), |
| | | | 10.60 (b, 1H) |
| 38 | 1-ethyl-3-{5-[2-(4-methyl-1,3- | 442.2 | 1.10 (t, 3H), 2.25 (s, 3H), 3.20 (qn, |
| | thiazol-5-yl)ethoxy]-6-pyrimidin-5- | | 2H), 3.25 (t, 2H), 4.55 (t, 2H), |
| | yl[1,3]thiazolo[5,4-b]pyridin-2- | | 6.70 (t, 1H), 8.15 (s, 1H), 8.80 (s, 1H), |
| | yl}urea | | 8.98 (s, 2H), 9.20 (s, 1H), 10.75 (b, |
| | | | 1H) |
| 39 | 1-ethyl-3-[6-pyrimidin-5-yl-5- | 399.1 | 1.10 (t, 3H), 3.20 (qn, 2H), 5.10 (q, |
| | (2,2,2- | | 2H), 6.73 (t, 1H), 8.28 (s, 1H), |
| | trifluoroethoxy)[1,3]thiazolo[5,4- | | 9.08 (s, 2H), 9.20 (s, 1H), 10.85 (b, 1H). |
| | b]pyridin-2-yl]urea | | |
| 40 | 1-ethyl-3-[6-(6-fluoropyridin-3-yl)- | 418.1 | 1.10 (t, 3H), 1.60-1.70 (m, 1H), |
| | 5-(tetrahydrofuran-3- | | 1.90-2.02 (m, 1H), 2.55-2.65 (m, |
| | ylmethoxy)[1,3]thiazolo[5,4- | | 1H), 3.20 (qn, 2H), 3.50 (dd, 1H), |
| | b]pyridin-2-yl]urea | | 3.65 (qn, 1H), 3.65-3.75 (m, 2H), |
| | | | 4.25-4.40 (m, 2H), 6.68 (t, 1H), |
| | | | 7.30 (dd, 1H), 8.05 (s, 1H), 8.22 (t, |
| | | | 1H), 8.48 (s, 1H), 10.70 (b, 1H) |
| 41 | 1-ethyl-3-{5-[2-(1- | 402.2 | 1.08 (d, 6H), 1.10 (t, 3H), 3.20 (qn, |
| | methylethoxy)ethoxy]-6-pyridin-3- | | 2H), 3.50-3.65 (m, 1H), 3.70 (t, |
| | yl[1,3]thiazolo[5,4-b]pyridin-2- | | 2H), 4.45 (t, 2H), 6.68 (t, 1H), |
| | yl}urea | | 7.42-7.48 (m, 1H), 8.05 (s, 1H), |
| | | | 8.05-8.10 (m, 1H), 8.55 (d, 1H), |
| | | | 8.90 (s, 1H), 10.70 (b, 1H) |
| 42 | 1-ethyl-3-[6-pyridin-3-yl-5- | 400.1 | 1.10 (t, 3H), 1.60-1.70 (m, 1H), |
| | (tetrahydrofuran-3- | | 1.90-2.02 (m, 1H), 2.55-2.65 (m, |
| | ylmethoxy)[1,3]thiazolo[5,4- | | 1H), 3.20 (qn, 2H), 3.50 (dd, 1H), |
| | b]pyridin-2-yl]urea | | 3.65 (qn, 1H), 3.65-3.75 (m, 2H), |
| | | | 4.25-4.40 (m, 2H), 6.68 (t, 1H), |
| | | | 7.45-7.50 (m, 1H), 7.95-8.05 (m, |
| | | | 1H), 8.05 (s, 1H), 8.55 (d, 1H), |
| | | | 8.80 (s, 1H), 10.70 (b, 1H) |
| 43 | 1-ethyl-3-[5-(2-methylpropoxy)-6- | 373.1 | 0.95 (d, 6H), 1.10 (t, 3H), |
| | pyrimidin-5-yl[1,3]thiazolo[5,4- | | 1.95-2.00 (m, 1H), 3.20 (qn, 2H), |
| | b]pyridin-2-yl]urea | | 4.15 (d, 2H), 6.69 (t, 1H), 8.19 (s, 1H), |
| | | | 9.08 (s, 2H), 9.18 (s, 1H), 10.70 (b, |
| | | | 1H) |
| 44 | 1-ethyl-3-[5-(2-methoxyethoxy)-6- | 375.1 | 1.10 (t, 3H), 3.20 (qn, 2H), 3.30 (s, |
| | pyrimidin-5-yl[1,3]thiazolo[5,4- | | 3H), 3.68 (t, 2H), 4.50 (t, 2H), |
| | b]pyridin-2-yl]urea | | 6.68 (t, 1H), 8.20 (s, 1H), 9.10 (s, 2H), |
| | | | 9.18 (s, 1H), 10.75 (b, 1H) |
| 45 | 1-ethyl-3-[5-(2-methoxyethoxy)-6- | 374.1 | 1.10 (t, 3H), 3.20 (qn, 2H), 3.30 (s, |
| | pyridin-3-yl[1,3]thiazolo[5,4- | | 3H), 3.68 (t, 2H), 4.50 (t, 2H), |
| | b]pyridin-2-yl]urea | | 6.68 (t, 1H), 7.45-7.50 (dd, 1H), 8.03 (s, |
| | | | 1H), 8.06 (t, 1H), 8.55 (dd, 1H), |
| | | | 8.82 (s, 1H), 10.70 (b, 1H) |
| 46 | 1-[5-(2-ethoxyethoxy)-6-pyrimidin- | 388.2 | 1.10 (t, 6H), 3.20 (qn, 2H), 3.45 (q, |
| | 5-yl[1,3]thiazolo[5,4-b]pyridin-2- | | 3H), 3.7 (t, 2H), 4.8 (t, 2H), 6.70 (t, |
| | yl]-3-ethylurea | | 1H), 8.21 (s, 1H), 9.12 (s, 2H), |
| | | | 9.17 (s, 1H), 10.80 (b, 1H) |
| 47 | 1-ethyl-3-{5-[2-(prop-2-en-1- | 401.2 | 1.10 (t, 3H), 3.20 (qn, 2H), 3.71 (t, |
| | yloxy)ethoxy]-6-pyrimidin-5- | | 2H), 3.95 (t, 2H), 4.49 (t, 2H), |
| | yl[1,3]thiazolo[5,4-b]pyridin-2- | | 5.1 (d, 1H), 5.17-5.21 (d, 1H), |
| | yl}urea | | 5.79-5.86 (m, 1H) 6.66 (t, 1H), (dd, 1H), |
| | | | 8.19 (s, 1H), 9.09 (s 2H), 9.15 (s, |
| | | | 1H), 10.77 (b, 1H) |
| 48 | 1-ethyl-3-[6-(6-fluoropyridin-3-yl)- | 391.8 | 1.10 (t, 3H), 3.20 (qn, 2H), 3.30 (s, |
| | 5-(2- | | 3H), 3.68 (t, 2H), 4.50 (t, 2H), |
| | methoxyethoxy)[1,3]thiazolo[5,4- | | 6.68 (t, 1H), 7.45-7.50 (dd, 1H), 8.03 (s, |
| | b]pyridin-2-yl]urea | | 1H), 8.06 (t, 1H), 8.55 (dd, 1H), |
| | | | 8.82 (s, 1H), 10.70 (b, 1H) |
| 49 | 1-ethyl-3-[6-pyrimidin-5-yl-5- | 415.8 | 1.10 (t, 3H), 1.25-1.40 (m, 2H), |
| | (tetrahydro-2H-pyran-4- | | 1.60 (dd, 2H), 1.95-2.05 (m, 1H), |
| | ylmethoxy)[1,3]thiazolo[5,4- | | 3.20 (qn, 2H), 3.30 (t, 2H), |
| | b]pyridin-2-yl]urea | | 3.85 (dd, 2H), 4.22 (d, 2H), 6.68 (t, 1H), |
| | | | 8.17 (s, 1H), 9.06 (s, 2H), 9.17 (s, |
| | | | 1H), 10.72 (b, 1H) |
| 50 | 1-ethyl-3-[6-pyrimidin-5-yl-5-(1,3- | 413.8 | 1.10 (t, 3H), 3.20 (qn, 2H), 5.60 (s, |
| | thiazol-4- | | 2H), 6.70 (t, 1H), 7.72 (s, 1H), |
| | ylmethoxy)[1,3]thiazolo[5,4- | | 8.22 (s, 1H), 9.08 (s, 2H), 9.10 (s, 1H), |
| | b]pyridin-2-yl]urea | | 9.12 (s, 1H), 10.75 (b, 1H). |
| 51 | 1-ethyl-3-[6-pyrimidin-5-yl-5- | 400.8 | 1.09 (t, 3H), 1.52-1.65 (m, 1H), |
| | (tetrahydrofuran-2- | | 1.75-1.95 (m, 3H), 3.19 (qn, 2H), |
| | ylmethoxy)[1,3]thiazolo[5,4- | | 3.25-3.45 (m, 1H), 3.60 (q, 1H), |
| | b]pyridin-2-yl]urea | | 3.75 (q, 1H), 4.08 (qn, 1H), 6.08 (t, |
| | | | 1H), 6.65 (t, 1H), 7.65 (s, 1H), |
| | | | 8.85 (s, 2H), 9.20 (s, 1H), 10.35 (b, 1H) |
| 53 | ethyl 5-{2-[(ethylcarbamoyl)amino]- | 472.3 | 1.10 (t, 3H), 1.36 (t, 3H), |
| | 5-(tetrahydrofuran-3- | | 1.62-1.73 (m, 1H), 1.93-2.04 (m, 1H), |
| | ylmethoxy)[1,3]thiazolo[5,4- | | 2.59-2.66 (m, 1H), 3.19 (qn, 2H), |
| | b]pyridin-6-yl}pyridine-3- | | 3.51 (dd, 1H), 3.60-3.73 (m, 2H), |
| | carboxylate | | 3.78 (t, 1H), 4.23 (dd, 1H), |
| | | | 4.30-4.42 (m, 3H), 6.68 (t, 1H), 8.17 (s, 1H), |
| | | | 8.56 (t, 1H), 9.03 (d, 1H), 9.07 (d, |
| | | | 1H), 10.74 (s, 1H) |
| 54 | 1-[6-(6-cyanopyridin-3-yl)-5- | 425.2 | 1.10 (t, 3H), 1.60-1.72 (m, 1H), |
| | (tetrahydrofuran-3- | | 1.95-2.04 (m, 1H), 2.59-2.68 (m, |
| | ylmethoxy)[1,3]thiazolo[5,4- | | 1H), 3.19 (qn, 2H), 3.50 (dd, 1H), |
| | b]pyridin-2-yl]-3-ethylurea | | 3.65 (m, 1H), 3.68-3.77 (m, 2H), |
| | | | 4.25-4.39 (m, 2H), 6.68 (t, 1H), |
| | | | 8.13 (d, 1H), 8.17 (s, 1H), 8.31 (dd, |
| | | | 1H), 9.00-9.01 (m, 1H), 10.65 (b, |
| | | | 1H) |
| 55 | (2E)-3-(3-{2- | 469.3 | 1.10 (t, 3H), 1.63-1.73 (m, 1H), |
| | [(ethylcarbamoyl)amino]-5- | | 1.93-2.02 (m, 1H), 2.56-2.67 (m, |
| | (tetrahydrofuran-3- | | 1H), 3.19 (qn, 2H), 3.52 (dd, 1H), |
| | ylmethoxy)[1,3]thiazolo[5,4- | | 3.58-3.63 (m, 1H), 3.66-3.76 (m, |
| | b]pyridin-6-yl}phenyl)prop-2-enoic | | 2H), 4.25 (dd, 1H), 4.35 (dd, 1H), |
| | acid | | 6.60 (d, 1H), 6.68 (t, 1H), 7.50 (t, |
| | | | 1H), 7.63 (d, 1H), 7.68 (d, 2H), |
| | | | 7.91 (s, 1H), 8.01 (s, 1H), 10.69 (b, |
| | | | 1H), 12.40 (b, 1H) |
| 56 | 1-ethyl-3-(5-methoxy-6-pyrimidin- | 330.9 | 1.10 (t, 3H), 3.19 (qn, 2H), 3.97 (s, |
| | 5-yl[1,3]thiazolo[5,4-b]pyridin-2- | | 3H), 6.70 (t, 1H), 8.17 (s, 1H), |
| | yl)urea | | 9.05 (s, 2H), 9.17 (s, 1H), 10.60 (b, 1H) |
| 57 | 1-[6-(5-cyanopyridin-3-yl)-5- | 425.3 | 1.10 (t, 3H), 1.63-1-71 (m, 1H), |
| | (tetrahydrofuran-3- | | 1.90-2.05 (m, 1H), 2.59-2.70 (m, |
| | ylmethoxy)[1,3]thiazolo[5,4- | | 1H), 3.19 (qn, 2H), 3.51 (dd, 1H), |
| | b]pyridin-2-yl]-3-ethylurea | | 3.65 (qn, 1H), 3.67-3.76 (m, 2H), |
| | | | 4.24-4.36 (m, 2H), 6.73 (t, 1H), |
| | | | 8.16 (s, 1H), 8.56 (t, 1H), 9.00 (d, |
| | | | 1H), 9.10 (d, 1H), 10.95 (b, 1H) |
| 59 | 1-ethyl-3-[6-(6-fluoropyridin-3-yl)- | 431.8 | 1.10 (t, 3H), 1.25-1.35 (m, 2H), |
| | 5-(tetrahydro-2H-pyran-4- | | 1.55-1.65 (m, 1H), 1.95 (m, 1H), |
| | ylmethoxy)[1,3]thiazolo[5,4- | | 3.20 (qn, 2H), 3.55 (dd, 2H), |
| | b]pyridin-2-yl]urea | | 3.8-3.9 (dd, 2H), 4.2 (d, 2H), 6.70 (t, |
| | | | 1H), 7.25-7.35 (dd, 1H), 8.05 (s, |
| | | | 1H), 8.15-8.25 (m, 1H) 8.45 (s, |
| | | | 1H), 10.70 (b, 1H) |
| 60 | 1-ethyl-3-[6-(5-fluoropyridin-3-yl)- | 431.8 | 1.10 (t, 3H), 1.25-1.35 (m, 2H), |
| | 5-(tetrahydro-2H-pyran-4- | | 1.55-1.65 (m, 1H), 1.95 (m, 1H), |
| | ylmethoxy)[1,3]thiazolo[5,4- | | 3.20 (qn, 2H), 3.45 (dd, 2H), |
| | b]pyridin-2-yl]urea | | 3.8-3.9 (dd, 2H), 4.25 (d, 2H), 6.70 (t, |
| | | | 1H), 7.95-8.05 (dd, 1H), 8.15 (s, |
| | | | 1H), 8.55 (d, 1H) 8.75 (d, 1H), |
| | | | 10.70 (b, 1H) |
| 61 | 1-ethyl-3-[6-(2-methoxypyridin-3- | 443.8 | 1.10 (t, 3H), 1.15-1.30 (m, 3H), |
| | yl)-5-(tetrahydro-2H-pyran-4- | | 1.45-1.52 (m, 2H), 1.80-1.92 (m, |
| | ylmethoxy)[1,3]thiazolo[5,4- | | 1H), 3.15-3.25 (m, 1H), 3.20 (qn, |
| | b]pyridin-2-yl]urea | | 2H), 3.77-3.82 (m, 2H), 3.80 (s, |
| | | | 3H), 4.15 (d, 2H), 6.65 (t, 1H), |
| | | | 7.08 (dd, 1H), 7.69 (dd, 1H), |
| | | | 7.82 (s, 1H), 8.20 (dd, 1H), 10.62 (b, |
| | | | 1H) |
| 62 | 1-[6-(3,5-dimethylisoxazol-4-yl)-5- | 431.8 | 1.10 (t, 3H), 1.25-1.35 (m, 2H), |
| | (tetrahydro-2H-pyran-4- | | 1.55-1.65 (m, 1H), 1.95 (m, 1H), |
| | ylmethoxy)[1,3]thiazolo[5,4- | | 2.13 (s, 3H), 2.30 (s, 3H), 3.20 (qn, |
| | b]pyridin-2-yl]-3-ethylurea | | 2H), 3.35 (dd, 2H), 3.78-3.9 (dd, |
| | | | 2H), 4.20 (d, 2H), 6.70 (t, 1H), |
| | | | 7.85-7.95 (s, 1H), 10.70 (b, 1H) |
| 63 | 1-ethyl-3-{6-pyrimidin-5-yl-5-[(2R)- | 400.9 | 1.10 (t, 3H), 1.60-1.70 (m, 1H), |
| | tetrahydrofuran-2- | | 1.70-1.85 (m, 1H), 1.85-2.05 (m, |
| | ylmethoxy][1,3]thiazolo[5,4- | | 1H), 3.20 (qn, 2H), 3.6-3.8 (m, |
| | b]pyridin-2-yl}urea | | 2H), 4.15-4.25 (m, 1H), |
| | | | 4.3-4.45 (m, 2H), 6.70 (t, 1H), 8.2 (s, 1H), |
| | | | 9.08 (s, 2H), 9.19 (s, 1H), 10.70 (b, |
| | | | 1H) |
| 64 | 1-ethyl-3-[6-pyrimidin-5-yl-5- | 386.9 | 1.10 (t, 3H), 1.95-2.1 (m, 1H), |
| | (tetrahydrofuran-3- | | 2.19-2.31 (m, 1H), 3.20 (qn, 2H), |
| | yloxy)[1,3]thiazolo[5,4-b]pyridin-2- | | 3.70-3.85 (m, 3H), 3.9-4.0 (dd, 1H), |
| | yl]urea | | 5.6-5.7 (t, 1H), 6.70 (t, 1H), 8.2 (s, 1H), |
| | | | 9.08 (s, 2H), 9.19 (s, 1H), 10.70 (b, |
| | | | 1H) |
| 66 | 1-[6-(2-cyanopyrimidin-5-yl)-5- | 425.8 | 1.10 (t, 3H), 1.62-1.73 (m, 1H), |
| | (tetrahydrofuran-3- | | 1.92-2.05 (m, 1H), 2.62-2.75 (m, |
| | ylmethoxy)[1,3]thiazolo[5,4- | | 1H), 3.20 (qn, 2H), 3.52 (dd, 1H), |
| | b]pyridin-2-yl]-3-ethylurea | | 3.65 (dd, 1H), 3.75 (dd, 2H), |
| | | | 4.28-4.42 (m, 2H), 6.68 (t, 1H), 8.30 (s, |
| | | | 1H), 9.32 (s, 2H), 10.78 (b, 1H) |
| 68 | 1-ethyl-3-[6-pyrimidin-5-yl-5- | 401.1 | 1.10 (t, 3H), 1.60-1.70 (m, 1H), |
| | (tetrahydrofuran-3- | | 1.90-2.02 (m, 1H), 2.58-2.70 (m, |
| | ylmethoxy)[1,3]thiazolo[5,4- | | 1H), 3.19 (qn, 2H), 3.55 (dd, 1H), |
| | b]pyridin-2-yl]urea (Chiral isomer 1) | | 3.65 (qn, 1H), 3.70 (q, 2H), |
| | | | 4.25-4.40 (m, 2H), 6.70 (t, 1H), 8.18 (s, |
| | | | 1H), 9.06 (s, 2H), 9.17 (s, 1H), |
| | | | 10.60 (b, 1H) |
| 69 | 1-ethyl-3-[6-pyrimidin-5-yl-5- | 401.1 | 1.10 (t, 3H), 1.60-1.70 (m, 1H), |
| | (tetrahydrofuran-3- | | 1.90-2.02 (m, 1H), 2.58-2.70 (m, |
| | ylmethoxy)[1,3]thiazolo[5,4- | | 1H), 3.19 (qn, 2H), 3.55 (dd, 1H), |
| | b]pyridin-2-yl]urea (Chiral isomer 2) | | 3.65 (qn, 1H), 3.70 (q, 2H), |
| | | | 4.25-4.40 (m, 2H), 6.70 (t, 1H), 8.18 (s, |
| | | | 1H), 9.06 (s, 2H), 9.17 (s, 1H), |
| | | | 10.60 (b, 1H) |
| 70 | 1-ethyl-3-{5-(tetrahydrofuran-3- | 467.8 | 1.10 (t, 3H), 1.60-1.70 (m, 1H), |
| | ylmethoxy)-6-[6- | | 1.90-2.30 (m, 1H), 2.58-2.70 (m, |
| | (trifluoromethyl)pyridin-3- | | 1H), 3.20 (qn, 2H), 3.50 (dd, 1H), |
| | yl][1,3]thiazolo[5,4-b]pyridin-2- | | 3.60-3.70 (m, 1H), 3.70-3.80 (m, |
| | yl}urea | | 2H), 4.25-4.40 (m, 2H), 6.70 (t, |
| | | | 1H), 8.00 (d, 1H), 8.15 (s, 1H), |
| | | | 8.33 (dd, 1H), 9.00 (s, 1H), |
| | | | 10.77 (b, 1H) |
| 71 | 1-ethyl-3-[6-(1-methyl-1H-pyrazol- | 402.9 | 1.10 (t, 3H), 1.68-1.80 (m, 1H), |
| | 4-yl)-5-(tetrahydrofuran-3- | | 2.02-2.14 (m, 1H), 2.72-2.83 (m, |
| | ylmethoxy)[1,3]thiazolo[5,4- | | 1H), 3.20 (qn, 2H), 3.58-3.63 (m, |
| | b]pyridin-2-yl]urea | | 1H), 3.69 (dd, 1H), 3.80-4.42 (m, |
| | | | 2H), 4.41 (s, 3H), 4.29-4.41 (m, |
| | | | 2H), 6.66 (t, 1H), 8.02 (s, 1H), |
| | | | 8.16 (s, 1H), 8.21 (s, 1H), 10.61 (b, 1H) |
| 75 | 1-[6-(2-cyanopyrimidin-5-yl)-5- | 411.9 | 1.10 (t, 3H), 2.00-2.10 (m, 1H), |
| | (tetrahydrofuran-3- | | 2.20-2.33 (m, 1H), 3.20 (qn, 2H), |
| | yloxy)[1,3]thiazolo[5,4-b]pyridin-2- | | 3.78-3.82 (m, 2H), 3.80-3.98 (m, |
| | yl]-3-ethylurea | | 2H), 5.65-5.70 (m, 1H), 6.70 (t, |
| | | | 1H), 8.30 (s, 1H), 9.30 (s, 2H), |
| | | | 10.85 (b, 1H) |
| 76 | 1-ethyl-3-[6-(6-fluoropyridin-3-yl)- | 403.9 | 1.10 (t, 3H), 1.97-2.60 (m, 1H), |
| | 5-(tetrahydrofuran-3- | | 2.20-2.30 (m, 1H), 3.20 (qn, 2H), |
| | yloxy)[1,3]thiazolo[5,4-b]pyridin-2- | | 3.72-3.82 (m, 3H), 3.95 (dd, 1H), |
| | yl]urea | | 5.60-5.65 (m, 1H), 6.72 (t, 1H), |
| | | | 7.28 (dd, 1H), 8.08 (s, 1H), |
| | | | 8.19-8.25 (m, 1H), 8.48 (s, 1H), |
| | | | 10.78 (b, 1H) |
| 77 | 1-ethyl-3-{5-(tetrahydrofuran-3- | 468.8 | 1.10 (t, 3H), 1.61-1.72 (m, 1H), |
| | ylmethoxy)-6-[2- | | 1.92-2.04 (m, 1H), 2.60-2.72 (m, |
| | (trifluoromethyl)pyrimidin-5- | | 1H), 3.20 (qn, 2H), 3.51 (dd, 1H), |
| | yl][1,3]thiazolo[5,4-b]pyridin-2- | | 3.62 (dd, 1H), 3.68-3.77 (m, 2H), |
| | yl}urea | | 4.28-4.40 (m, 2H), 6.70 (t, 1H), |
| | | | 8.30 (s, 1H), 9.35 (s, 2H), |
| | | | 10.80 (1H) |
| 78 | 1-ethyl-3-{6-[5-(5-oxo-4,5-dihydro- | 483.8 | 1.10 (t, 3H), 1.62-1.73 (m, 1H), |
| | 1,3,4-oxadiazol-2-yl)pyridin-3-yl]-5- | | 1.92-2.05 (m, 1H), 2.58-2.69 (m, |
| | (tetrahydrofuran-3- | | 1H), 3.20 (qn, 2H), 3.54 (dd, 1H), |
| | ylmethoxy)[1,3]thiazolo[5,4- | | 3.62 (dd, 1H), 3.67-3.80 (m, 2H), |
| | b]pyridin-2-yl}urea | | 4.25-4.40 (m, 2H), 6.68 (t, 1H), |
| | | | 8.18 (s, 1H), 8.41 (t, 1H), 8.95 (d, |
| | | | 1H), 8.97 (d, 1H), 10.76 (b, 1H), |
| | | | 12.40 (b, 1H) |
| 79 | 1-ethyl-3-[6-(5-fluoropyridin-3-yl)- | 417.9 | 1.10 (t, 3H), 1.62-1.73 (m, 1H), |
| | 5-(tetrahydrofuran-3- | | 1.92-2.05 (m, 1H), 2.58-2.69 (m, |
| | ylmethoxy)[1,3]thiazolo[5,4- | | 1H), 3.20 (qn, 2H), 3.52 (dd, 1H), |
| | b]pyridin-2-yl]urea | | 3.62 (qn, 1H), 3.69-3.78 (m, 2H), |
| | | | 4.24-4.39 (m, 2H), 6.68 (t, 1H), |
| | | | 8.00 (dd, 1H), 8.12 (s, 1H), |
| | | | 8.58 (dd, 1H), 8.71 (s, 1H), 10.73 (b, |
| | | | 1H) |

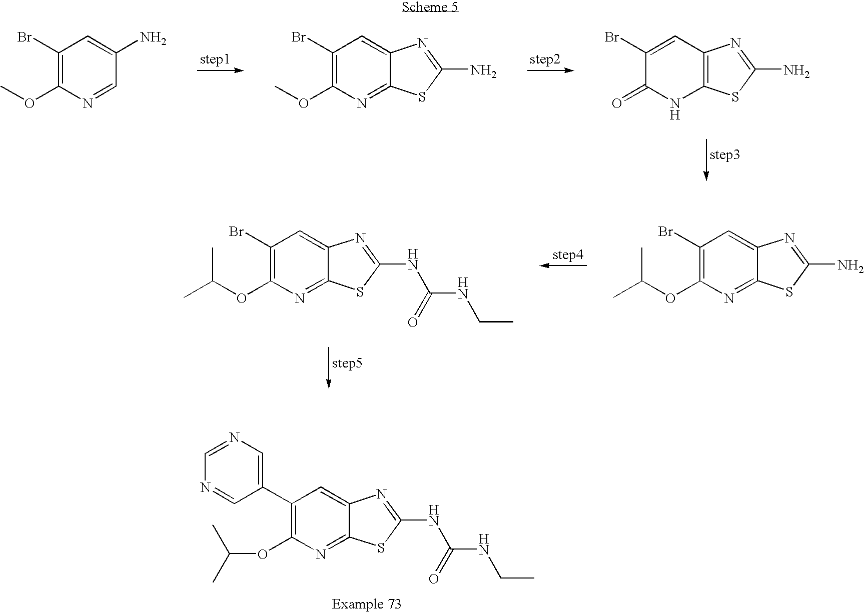

### Step 1: 6-bromo-5-methoxythiazolo[5,4-b]pyridin-2-amine (Intermediate 19)

In a 250 mL RB flask, 5-bromo-6-methoxypyridin-3-amine (5 g, 24.63 mmol) was added to a solution of potassium thioacetate (20 g, 175.12 mmol) in acetic acid (100 ml) at 0° C. To this mixture, bromine (2.5 ml, 48.53 mmol) solution in acetic acid (10 ml) was added slowly maintaining temperature near 0° C. Stirring was continued for another 5 h at RT. Then pH of reaction mixture was adjusted to 5 with 6 N sodium hydroxide solution at 0° C. Reaction mixture was extracted with ethyl acetate (3 times). Ethyl acetate layers were combined, washed with brine, dried on anhydrous sodium sulphate and concentrated to afford 6-bromo-5-methoxythiazolo[5,4-b]pyridin-2-amine (4.80 g, 74.9%) as yellow solid.

MS (ES⁺): 260.8 for C₇H₆BrN₃OS

¹H NMR (DMSO D₆)δ: 3.90 (s, 3H), 7.68 (b, 2H), 7.92 (s, 1H)

### Step 2: 2-amino-6-bromothiazolo[5,4-b]pyridin-5(4H)-one (Intermediate 20)

A suspension of 6-bromo-5-methoxythiazolo[5,4-b]pyridin-2-amine (1 g, 3.84 mmol) in 46% HBr solution (18 ml, 152.48 mmol) was heated at reflux for 2.5 hr. Reaction mixture was cooled to room temperature. Excess HBr solution was decanted off and remaining slurry was neutralized with saturated aqueous sodium bicarbonate solution at 0° C. Solid obtained was filtered, washed with chilled water and dried under vacuum to afford 2-amino-6-bromothiazolo[5,4-b]pyridin-5(4H)-one (0.750 g, 79%) as a white solid.

MS (ES⁺): 247.8 for C₆H₄BrN₃OS

¹H NMR (DMSO D₆)δ: 7.51 (b, 2H), 7.85 (s, 1H), 11.40 (b, 1H)

### Step 3: 6-bromo-5-isopropoxythiazolo[5,4-b]pyridin-2-amine (Intermediate 21)

In a 50 mL round-bottomed flask 2-amino-6-bromothiazolo[5,4-b]pyridin-5(4H)-one (1 g, 4.06 mmol) was dissolved in dry DMF (6 mL) under N₂. To the solution, cesium carbonate (1.589 g, 4.88 mmol) was added in one portion at 40° C. After 5 minutes 2-bromopropane (0.534 mL, 5.69 mmol) was added. The resulting mixture was stirred at 40° C. for 3 hr. Reaction was cooled to RT. DMF was evaporated and residue was diluted with water, extracted with ethyl acetate thrice. Ethyl acetate layers were combined, dried on anhydrous Na2SO4 and concentrated under vacuum. Solid residue was purified by flash chromatography using ethyl acetate and hexane as eluent. Pure fractions were combined and dried to afford pure 6-bromo-5-isopropoxythiazolo[5,4-b]pyridin-2-amine (0.490 g, 41.8%) as off-white solid.

MS (ES⁺): 288.8 for C₉H₁₀BrN₃OS

¹H NMR (DMSO D₆)δ: 1.30 (d, 6H), 5.08-5.22 (m, 1H), 7.58 (b, 2H), 7.90 (s, 1H)

### Step 4: 1-(6-bromo-5-isopropoxythiazolo[5,4-b]pyridin-2-yl)-3-ethylurea (Intermediate 22)

Same as in Step 4 of scheme 4 using intermediate 21 as staring material.

Yield 76%

MS (ES⁺): 359.8 for C₁₂H₁₅BrN₄O₂S

¹H NMR (DMSO D₆) δ: 1.09 (t, 3H), 1.35 (d, 6H), 3.18 (qn, 2H), 5.15-5.25 (m, 1H), 6.66 (t, 1H), 8.23 (s, 1H), 10.72 (b, 1H)

### Step 5: 1-ethyl-3-[5-(1-methylethoxy)-6-pyrimidin-5-yl[1,3]thiazolo[5,4-b]pyridin-2-yl]urea (Example 73)

Same as in Step 4 of scheme 4 using intermediate 22 as staring material.

Yield 20%

MS (ES⁺): 358.9 for C₁₆H₁₈N₆O₂S

¹H NMR (DMSO D₆) δ: 1.01 (t, 3H), 1.32 (d, 6H), 3.18 (qn, 2H), 5.5.30-5.40 (m, 1H), 6.73 (t, 1H), 8.15 (s, 1H), 9.06 (s, 2H), 9.16 (s, 1H), 10.75 (b, 1H)

Following examples were made using protocol described under Scheme 5

| | | MS | |
|---|---|---|---|
| Example | Compound | (ES⁺): | ¹HNMR (DMSO-d6) δ |
| 72 | 1-[5-(cyclohex-2-en-1-yloxy)- | 413.8 | 1.08 (t, 3H), 1.70-1.85 (m, 1H), |
| | 6-(6-fluoropyridin-3- | | 1.90-2.05 (m, 3H), 2.15-2.25 (m, 2H), |
| | yl)[1,3]thiazolo[5,4-b]pyridin- | | 3.17 (qn, 2H), 5.70 (d, 1H), 5.95-6.05 (m, |
| | 2-yl]-3-ethylurea | | 1H), 6.17-6.22 (m, 1H), 6.58 (t, 1H), |
| | | | 7.22 (dd, 1H), 8.08 (s, 1H), |
| | | | 8.32-8.38 (m, 1H), 8.56 (s, 1H), 10.59 (b, 1H) |
| 74 | 1-ethyl-3-[6-(6-fluoropyridin-3- | 375.9 | 1.1 (t, 3H), 1.31 (d, 6H), 3.19 (qn, 2H), |
| | yl)-5-(1- | | 5.30-5.40 (m, 1H), 6.68 (t, 1H), |
| | methylethoxy)[1,3]thiazolo[5,4- | | 7.27 (dd, 1H), 8.04 (s, 1H), 8.18-8.26 (m, |
| | b]pyridin-2-yl]urea | | 1H), 8.45 (s, 1H), 10.68 (b, 1H) |

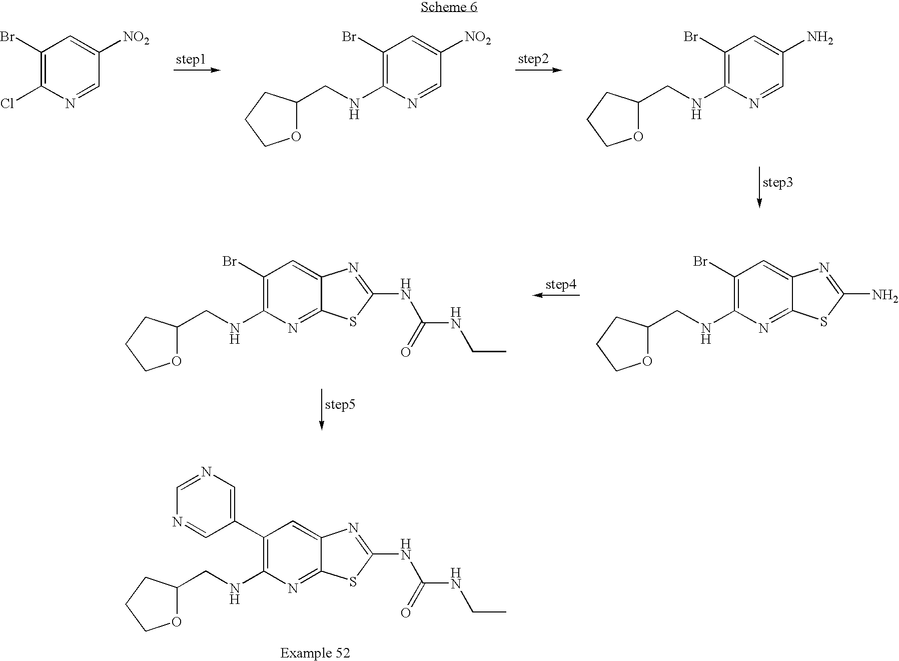

### Step 1: 3-bromo-5-nitro-N-((tetrahydrofuran-2-yl)methyl)pyridin-2-amine (intermediate 23)

To a stirred solution of 3-bromo-2-chloro-5-nitropyridine (2 g, 8.42 mmol) and (tetrahydrofuran-2-yl)methanamine (1.704 g, 16.85 mmol) in DMF (10 mL), potassium carbonate (2.328 g, 16.85 mmol) was added portion-wise and the mixture was stirred at 60° C. for 3 hr. RM cooled to RT, diluted with Ethyl Acetate (150-200 ml), washed with water 2 times and then with brine. Ethyl acetate layer was then dried over sodium sulfate and concentrated under vacuum to give crude product. 3-bromo-5-nitro-N-((tetrahydrofuran-2-yl)methyl)pyridin-2-amine (2.00 g, 79%) as brown gum.

MS (ES⁺): 302.8 for C₁₀H₁₂BrN₃O₃

### Step 2: 3-bromo-N2-((tetrahydrofuran-2-yl)methyl)pyridine-2,5-diamine (intermediate 24)

Similar to as described for step 2 of Scheme 4 using intermediate 23 as starting material.

Yield=61%

MS (ES⁺): 272.9 for C₁₀H₁₄BrN₃O

### Step 3: 6-bromo-N5-((tetrahydrofuran-2-yl)methyl)thiazolo[5,4-b]pyridine-2,5-diamine (intermediate 25)

Similar to as described for step 3 of Scheme 4 using intermediate 24 as starting material.

Yield=45%

MS (ES⁺): 330.8 for C₁₁H₁₃BrN₄OS

### Step 4: 1-(6-bromo-5-((tetrahydrofuran-2-yl)methylamino)thiazolo[5,4-b]pyridin-2-yl)-3-ethylurea (intermediate 26)

Similar to as described for step 4 of Scheme 4 using intermediate 25 as starting material.

Yield=57%

MS (ES⁺): 400.7 for C₁₄H₁₈BrN₅O₂S

### Example 52

### Step 5: 1-ethyl-3-{6-pyrimidin-5-yl-5-[(tetrahydrofuran-2-ylmethyl)amino][1,3]thiazolo[5,4-b]pyridin-2-yl}urea

Similar to as described for step 5 of Scheme 4 using intermediate 26 as starting material.

Yield=57%

MS (ES⁺): 399.9 for C₁₈H₂₁N7O₂S

¹H NMR (DMSO D₆)δ: 1.10 (t, 3H), 1.60-1.73 (m, 1H), 1.80 (qn, 2H), 1.90-2.01 (m, 1H), 3.20 (qn, 2H), 3.60-3.75 (m, 2H), 4.12-4.22 (m, 1H), 4.28-4.40 (m, 2H), 6.68 (t, 1H), 8.20 (s, 1H), 9.10 (s, 2H), 9.15 (s, 1H), 10.72 (b, 1H)

### Example 65

### Ethyl N-{2-[(ethylcarbamoyl)amino]-6-pyrimidin-5-yl[1,3]thiazolo[5,4-b]pyridin-5-yl}-L-alaninate

was prepared using similar protocol as described in Scheme 6

MS (ES⁺): 415.9 for C₁₈H₂₁N₇O₃S

¹H NMR (DMSO D₆)δ: 1.10 (t, 3H), 1.15 (t, 3H), 1.35 (d, 3H) 3.20 (qn, 2H), 4.0-4.2 (m, 2H), 4.5 (qn, 1H), 6.45 (d, 1H), 6.70 (t, 1H), 7.7 (s, 1H), 8.9 (s, 2H), 9.2 (s, 1H), 10.5 (b, 1H)

### Example 67

### (2S)-2-[[2-(ethylcarbamoylamino)-6-pyrimidin-5-yl-thiazolo[4,5-e]pyridin-5-yl]amino]-N-methyl-propanamide

In 25 ml rb flask ethyl 2-(2-(3-ethylureido)-6-(pyrimidin-5-yl)thiazolo[5,4-b]pyridin-5-ylamino)propanoate (60 mg, 0.14 mmol) was dissolved in 40% methyl amine solution (2 mL) and stirred for 2 hr. A precipitate was formed in the reaction. Precipitate was filtered, dried and triturated in acetonitrile to afford pure 2-(2-(3-ethylureido)-6-(pyrimidin-5-yl)thiazolo[5,4-b]pyridin-5-ylamino)-N-methylpropanamide (30.0 mg, 51.9%) as white solid.

MS (ES⁺): 400.9 for C₁₇H₂₀N₈O₂S

¹H NMR (DMSO D₆)δ: 1.10 (t, 3H), 1.28 (d, 3H), 2.58 (d, 3H), 3.19 (qn, 2H), 4.48 (qn, 1H), 6.01 (d, 1H), 6.62 (t, 1H), 7.70 (s, 1H), 7.80 (q, 1H), 8.98 (s, 2H), 9.22 (s, 1H), 10.45 (b, 1H)

### Example 58

### 1-ethyl-3-(6-(3-hydroxypyrrolidin-1-yl)-5-((tetrahydrofuran-3-yl)methoxy)thiazolo[5,4-b]pyridin-2-yl)urea

In a 50 ml round bottom flask, tetrahydrofuran (20 mL) was added to a mixture of 1-(6-bromo-5-((tetrahydrofuran-3-yl)methoxy)thiazolo[5,4-b]pyridin-2-yl)-3-ethylurea (150 mg, 0.37 mmol), pyrrolidin-3-ol (65.1 mg, 0.75 mmol), Pd₂(dba)₃ (68.5 mg, 0.07 mmol), Xantphos (87 mg, 0.15 mmol). To this slurry, lithium bis(trimethylsilyl)amide (3.74 mL, 3.74 mmol) was added dropwise at 0° C. and the solution was refluxed at 71° C. overnight for 20 hrs. The reaction mixture was concentrated and residue was purified on reverse phase HPLC to afford 1-ethyl-3-(6-(3-hydroxypyrrolidin-1-yl)-5-((tetrahydrofuran-3-yl)methoxy)thiazolo[5,4-b]pyridin-2-yl)urea (62.0 mg, 40.7%) as off-white crystalline solid.

MS (ES⁺): 407.9 for C₁₈H₂₅N₅O₄S

¹H NMR (DMSO D₆)δ: 1.10 (t, 3H), 1.65-1.85 (m, 2H), 1.95-2.10 (m, 2H), 2.62-2.78 (m, 1, H), 3.12-3.22 (m, 2H), 3.19 (qn, 2H), 3.42 (qn, 1H), 3.55-3.63 (m, 2H), 3.69 (t, 1H), 3.74-3.88 (m, 2H), 4.14-4.32 (m, 2H), 4.35 (b, 1H), 4.87 (b, 1H), 6.80 (t, 1H), 7.08 (s, 1H), 10.55 (b, 1H)

### Enzyme Potency Testing Methods

Compounds may be tested for inhibition of GyrB ATPase activity using an ammonium molybdate/malachite green-based phosphate detection assay (Lanzetta, P. A., L. J. Alvarez, P. S. Reinach, and O. A. Candia, 1979, 100: 95-97). Assays can be performed in multiwell plates in 100 μl reactions containing: 50 mM HEPES buffer pH 7.5, 75 mM ammonium acetate, 5.5 mM magnesium chloride, 0.5 mM ethylenediaminetetraacetic acid, 5% glycerol, 1 mM 1,4-Dithio-DL-threitol, 200 nM bovine serum albumin, 5 μg/ml sheared salmon sperm DNA, 2.5 nM E. coli GyrA, 2.5 nM E. coli GyrB, 250 μM ATP, and compound in dimethylsulfoxide. Reactions can be quenched with 150 μl A of ammonium molybdate/malachite green detection reagent containing 1.2 mM malachite green hydrochloride, 8.5 mM ammonium molybdate tetrahydrate, and 1 M hydrochloric acid. Plates can be read in an absorbance plate reader at 650 nm and percent inhibition values may be calculated using dimethylsulfoxide (2%)-containing reactions as 0% inhibition and novobiocin-containing (2 μM) reactions as 100% inhibition controls. Compound potency can be based on IC₅₀ measurements determined from reactions performed in the presence of 10 different compound concentrations.

Compounds may be tested for inhibition of topoisomerase IV ATPase activity as described above for GyrB except the 100 μl reactions may contain the following: 20 mM TRIS buffer pH 8, 50 mM ammonium acetate, 8 mM magnesium chloride, 5% glycerol, 5 mM 1,4-Dithio-DL-threitol, 0.005% Brij-35, 5 μg/ml sheared salmon sperm DNA, 2.5 nM E. coli ParC, 2.5 nM E. coli ParE, 160 μM ATP, and compound in dimethylsulfoxide. Compound potency may be based on IC₅₀ measurements determined from reactions performed in the presence of 10 different compound concentrations.

### Msmegmatis GyrB Enzyme Assay

Compounds may be tested for inhibition of GyrB ATPase activity using an ammonium molybdate/malachite green-based phosphate detection assay (Lanzetta, P. A., L. J. Alvarez, P. S. Reinach, and O. A. Candia, 1979, 100: 95-97; Innova Biosciences malachite green detection kit). Assays can be performed in multi-well plates in 50 μl reactions containing: 50 mM HEPES buffer pH 7.7, 250 mM potassium glutamate, 200 mM potassium chloride, 2 mM magnesium chloride, 2% glycerol, 1 mM 1,4-Dithio-DL-threitol, 0.005% Brij-35, 15 nM Msm. GyrB, 650 μM ATP, and compound in dimethyl sulfoxide. Reactions can be quenched with 12.5 μl of ammonium molybdate/malachite green detection reagent (Pi color lock gold & accelerator mix; Innova Biosciences), followed by the addition of 5 μl of stabilizer (Innova Biosciences) after 5 min of incubation. Plates can be read in an absorbance plate reader at 650 nm after 30 min of incubation at room temperature and percent inhibition values may be calculated using dimethylsulfoxide (4%)-containing reactions as 0% inhibition and novobiocin-containing (1 μM) reactions as 100% inhibition controls. Compound potency can be based on IC₅₀ measurements determined from reactions performed in the presence of 10 different compound concentrations.

### Bacterial Susceptibility Testing Methods

Compounds may be tested for antimicrobial activity by susceptibility testing in liquid media. Compounds may be dissolved in dimethylsulfoxide and tested in 10 doubling dilutions in the susceptibility assays. The organisms used in the assay may be grown overnight on suitable agar media and then suspended in a liquid medium appropriate for the growth of the organism. The suspension can be a 0.5 McFarland and a further 1 in 10 dilution can be made into the same liquid medium to prepare the final organism suspension in 100 μL. Plates can be incubated under appropriate conditions at 37° C. for 24 hrs prior to reading. The Minimum Inhibitory Concentration (MIC) may be determined as the lowest drug concentration able to reduce growth by 80% or more.

### Mycobacteria Susceptibility Testing Methods

Protocol for MIC testing: Microplate Alamar Blue Assay (Franzblau et al, 1998. J. Clin. Microbiol. 36: 362-366).

Two hundred microliters of sterile deionized water was added to all outer-perimeter wells of sterile 96-well plates to minimize evaporation of the medium in the test wells during incubation. Serial two-fold dilutions of the compounds in DMSO were made in another 96 well plate starting from 64 ug/ml to 0.5 ug/ml. 4 ul volumes of these were dispensed into the wells in rows B to G in columns 2 to 10 by using a multichannel pipette. 200 ul of M. tuberculosis culture diluted to a cell number of about 5×10⁵ cfu/ml was added to all the wells and the contents of the wells were mixed well. Three wells in column 11 served as drug-free (inoculum-only) controls. And 3 wells served as drug-free medium controls. The plates were incubated at 37 deg C. for 5 days. Fifty microliters of a freshly prepared 1:1 mixture of Alamar Blue (Accumed International, Westlake, Ohio) reagent and 10% Tween 80 was added to well B11. The plates were reincubated at 37° C. for 24 h. If well B11 turned pink, the reagent mixture was added to all wells in the microplate (if the well remained blue, the reagent mixture would be added to another control well and the result would be read on the following day). The microplates were re-incubated for an additional 24 h at 37° C., and the colors of all wells were recorded. A blue color in the well was interpreted as no growth, and a pink color was scored as growth.

The MIC was defined as the lowest drug concentration, which prevented a color change from blue to pink.

1. A compound of formula (I): 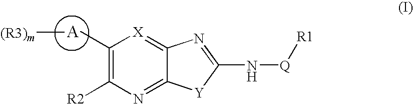
wherein
Y is S or O
Q is C(═O)NR⁴, C(═S)NR⁵, C(═O)O, C(═NH)NR⁶, C(═NCN)NR⁷, SO₂NR⁸, C(═O)C(═O)NR⁹, or C═O, SO₂;
to R⁴, R⁵, R⁶, R⁷, R⁸, R⁹ are independently selected from H, OH, C₁₋₄alkyl, and C₃₋₆ cycloalkyl;
R¹ is C₁₋₆alkyl, C₂₋₆alkenyl, C₂₋₆alkynyl, C₁₋₆alkoxy, C₁₋₆haloalkyl, C₁₋₆haloalkoxy, C₃₋₇ cycloalkyl, aryl, aryl C₁₋₆alkyl or heterocyclyl.
X is N or CRa wherein Ra is H, F, CH3, OCH3, CN;
m=0 to 5
Ring A is a carbocyclic or heterocyclic ring system comprising up to 12 ring atoms and up to 5 heteroatoms each independently selected from N, O and S; wherein if said heterocyclyl contains an —NH— moiety that nitrogen may be optionally substituted by a group R¹⁰;
R³ is hydrogen, halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N-(C₁₋₆alkyl)amino, N,N-(C₁₋₆alkyl)₂amino, C₁₋₆alkanoylamino, N-(C₁₋₆alkyl)carbamoyl, N,N-(C₁₋₆alkyl)₂carbamoyl, N-(C₁₋₆alkoxy)carbamoyl, N,N-(C₁₋₆alkoxy)₂carbamoyl, C₁₋₆alkylS(O)ₐ wherein a is 0 to 2, C₁₋₆alkoxycarbonyl, C₁₋₆alkoxycarbonylamino, N-(C₁₋₆alkyl)sulphamoyl, N,N-(C₁₋₆alkyl)₂sulphamoyl, C₁₋₆alkylsulphonylamino, carbocyclyl-R¹¹— or heterocyclyl-R¹²—; wherein R³ may be optionally substituted on carbon by one or more R¹³; and wherein if said heterocyclyl contains an —NH— moiety that nitrogen may be optionally substituted by a group selected from R¹⁴;
substituents on carbon are independently selected from halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N-(C₁₋₆alkyl)amino, N,N-(C₁₋₆alkyl)₂amino, C₁₋₆alkanoylamino, N-(C₁₋₆alkyl)carbamoyl, N,N-(C₁₋₆alkyl)₂carbamoyl, N-(C₁₋₆alkoxy)carbamoyl, N,N-(C₁₋₆alkoxy)₂carbamoyl, C₁₋₆alkylS(O)ₐ wherein a is 0 to 2, C₁₋₆alkoxycarbonyl, C₁₋₆alkoxycarbonylamino, N-(C₁₋₆alkyl)sulphamoyl, N,N-(C₁₋₆alkyl)₂sulphamoyl, C₁₋₆alkylsulphonylamino, carbocyclyl-R¹⁵— or heterocyclyl-R¹⁶—; and wherein if said heterocyclyl contains an —NH— moiety that nitrogen may be optionally substituted by a group selected from R¹⁷;
to and wherein R³ may be directly attached to the C5 position of thiazolopyridine or oxazolopyrdine without ring A, in which case R³ is halogen, cyano, C₁₋₆alkyl, C₂₋₆alkenyl, C₂₋₆alkynyl, C₁₋₆alkoxy, C₁₋₆haloalkyl, C₁₋₆haloalkoxy, C₃₋₇ cycloalkyl, C₃₋₇ cycloalkoxy, N-(C₁₋₆alkyl)amino, N,N-(C₁₋₆alkyl)₂amino, N-(C₁₋₆alkyl)amino alkoxy, N,N-(C₁₋₆alkyl)₂amino alkoxy, heterocycloalkoxy with 1-5 heteroatoms in it, arylalkoxy, heterocycloalkyl, arylalkyl, N-(C₁₋₆alkyl)aminoalkoxy, N,N-(C₁₋₆alkyl)₂aminoalkoxy, C₁₋₆alkylS(O)ₐ wherein a is 0 to 2, C₁₋₆alkoxycarbonyl, C₁₋₆alkoxycarbonylamino, N-(C₁₋₆alkyl)sulphamoyl, N,N-(C₁₋₆alkyl)₂sulphamoyl, C₁₋₆alkylsulphonylamino.
R¹¹, R¹⁵ and R¹⁶ are independently selected from a direct bond, —O—, —N(R¹⁸)—, —C(O)—, —N(R¹⁹)C(O)—, —C(O)N(R²⁰)—, —S(O)ₛ—, —SO₂N(R²¹)— or —N(R²²)SO₂—; wherein R¹⁸, R¹⁹, R²⁰, R²¹ and R²² are independently selected from hydrogen or C₁₋₆alkyl and s is 0-2; and
R¹⁰, R¹⁴ and R¹⁷ are independently selected from C₁₋₆alkyl, C₃₋₆cycloalkyl, C₁₋₆alkanoyl, C₁₋₆alkylsulphonyl, C₁₋₆alkoxycarbonyl, carbamoyl, N-(C₁₋₆alkyl)carbamoyl, N,N-(C₁₋₆alkyl)carbamoyl, benzyl, benzyloxycarbonyl, benzoyl and phenylsulphonyl;
R¹³ and R¹² are independently selected from halo, nitro, cyano, hydroxy, trifluoromethoxy, trifluoromethyl, amino, carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl;
R² is H, C₁₋₆alkyl, C₂₋₆alkenyl, C₂₋₆alkynyl, C₁₋₆alkoxy, C₁₋₆haloalkyl, C₁₋₆haloalkoxy, C₃₋₇ cycloalkyl, C₃₋₇ cycloalkoxy, N-(C₁₋₆alkyl)amino, N,N-(C₁₋₆alkyl)₂amino, N-(C₁₋₆alkyl)amino alkoxy, N,N-(C₁₋₆alkyl)₂amino alkoxy, heterocycloalkoxy with 1-5 heteroatoms in it, arylalkoxy, heterocycloalkyl, arylalkyl, N-(C₁₋₆alkyl)aminoalkoxy, N,N-(C₁₋₆alkyl)₂aminoalkoxy, C₁₋₆alkylS(O)ₐ wherein a is 0 to 2, C₁₋₆alkoxycarbonyl, C₁₋₆alkoxycarbonylamino, N-(C₁₋₆alkyl)sulphamoyl, N,N-(C₁₋₆alkyl)₂sulphamoyl, or C₁₋₆alkylsulphonylamino, or
R² is a group 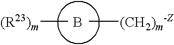
wherein
Z is O, S, or NR_{b} wherein R_{b} is H, C₁₋₆alkyl, C₃₋₇ cycloalkyl, C₁₋₆alkoxyC₁₋₆alkyl, cycloC₃₋₇alkoxyC₁₋₆alkyl; alternatively Z may represent a heterocyclic ring system comprising up to 7 ring atoms and up to 5 heteroatoms each independently selected from is N, O and S,
alternatively Z is absent and the R² group is directly attached to the thiazolopyridine or oxazolopyridine ring at the C6 position,
Ring B is a carbocyclic or heterocyclic ring system comprising up to 12 ring atoms and up to 5 heteroatoms each independently selected from N, O and S; and wherein if said ring system contains an —NH— moiety that nitrogen may be optionally substituted by a group R¹⁰;
R²³ is hydrogen, halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N-(C₁₋₆alkyl)amino, N,N-(C₁₋₆alkyl)₂amino, C₁₋₆alkanoylamino, N-(C₁₋₆alkyl)carbamoyl, N,N-(C₁₋₆alkyl)₂carbamoyl, N-(C₁₋₆alkoxy)carbamoyl, N,N-(C₁₋₆alkoxy)₂carbamoyl, C₁₋₆alkylS(O)ₐ wherein a is 0 to 2, C₁₋₆alkoxycarbonyl, C₁₋₆alkoxycarbonylamino, N-(C₁₋₆alkyl)sulphamoyl, N,N-(C₁₋₆alkyl)₂sulphamoyl, C₁₋₆alkylsulphonylamino, carbocyclyl-R¹¹— or heterocyclyl-R¹²—; wherein the carbocyclyl or heterocyclyl may be optionally substituted on carbon by one or more R¹³; and wherein if said heterocyclyl contains an —NH— moiety that nitrogen may be optionally substituted by a group R¹⁴;
or alternatively Ring B may be absent and R²³ is directly attached to —(CH₂)ₘ—, in which case R²³ is selected from halogen, cyano, C₁₋₆alkyl, C₂₋₆alkenyl, C₂₋₆alkynyl C₁₋₆alkoxy, C₁₋₆haloalkyl, C₁₋₆haloalkoxy, C₃₋₇ cycloalkyl, C₃₋₇ cycloalkoxy, N-(C₁₋₆alkyl)amino, N,N-(C₁₋₆alkyl)₂amino, N-(C₁₋₆alkyl)amino alkoxy, N,N-(C₁₋₆alkyl)₂amino alkoxy, heterocycloalkoxy with 1-5 heteroatoms in it, arylalkoxy, heterocycloalkyl, arylalkyl, N-(C₁₋₆alkyl)aminoalkoxy, N,N-(C₁₋₆alkyl)₂aminoalkoxy, C₁₋₆alkylS(O)ₐ wherein a is 0 to 2, C₁₋₆alkoxycarbonyl, C₁₋₆alkoxycarbonylamino, N-(C₁₋₆alkyl)sulphamoyl, N,N-(C₁₋₆alkyl)₂sulphamoyl, C₁₋₆alkylsulphonylamino;
or a pharmaceutically acceptable salt thereof.

2. A compound of formula I as claimed in claim 1 or a pharmaceutically acceptable salt thereof and wherein Q is any one of C(═O)NH, C(═S)NH, CO, C(═O)C(═O)NH.

3. A compound of formula I as claimed in claim 1 or a pharmaceutically acceptable salt thereof and wherein R¹ is any one of —CH₃, CH₂CH₃, CH(CH3)₂, CH₂CH(CH3)₂, OCH₃, CF₃CH₂, CH₂CH═CH₂, cyclopropyl, prolinyl, pyrazinyl, pyrimidinyl.

4. A compound of formula I as claimed in claim 1 or a pharmaceutically acceptable salt thereof and wherein X is any one of CH, CF, N.

5. A compound of formula I as claimed in claim 1 or a pharmaceutically acceptable salt thereof and wherein Ring A is any one of

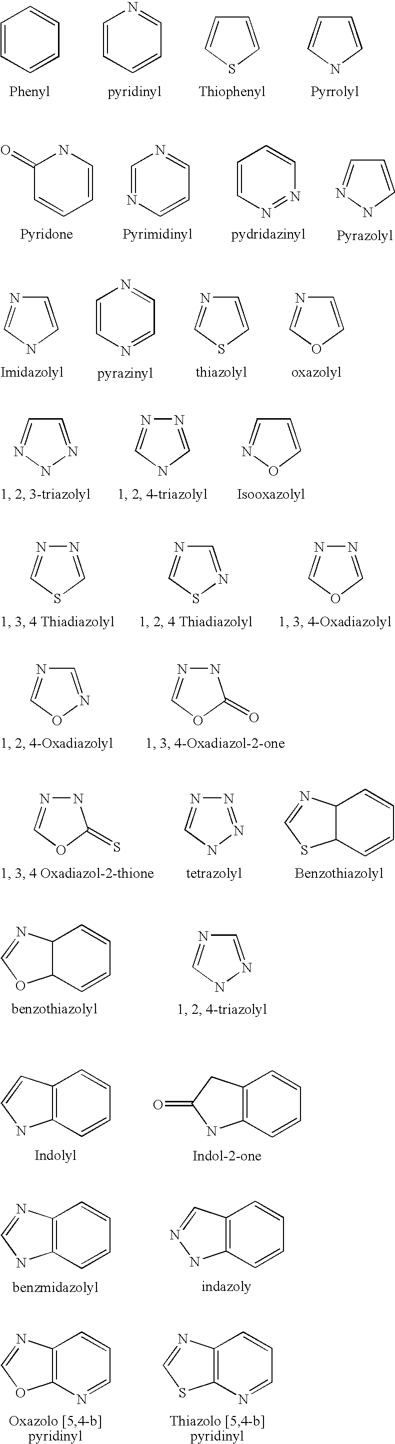

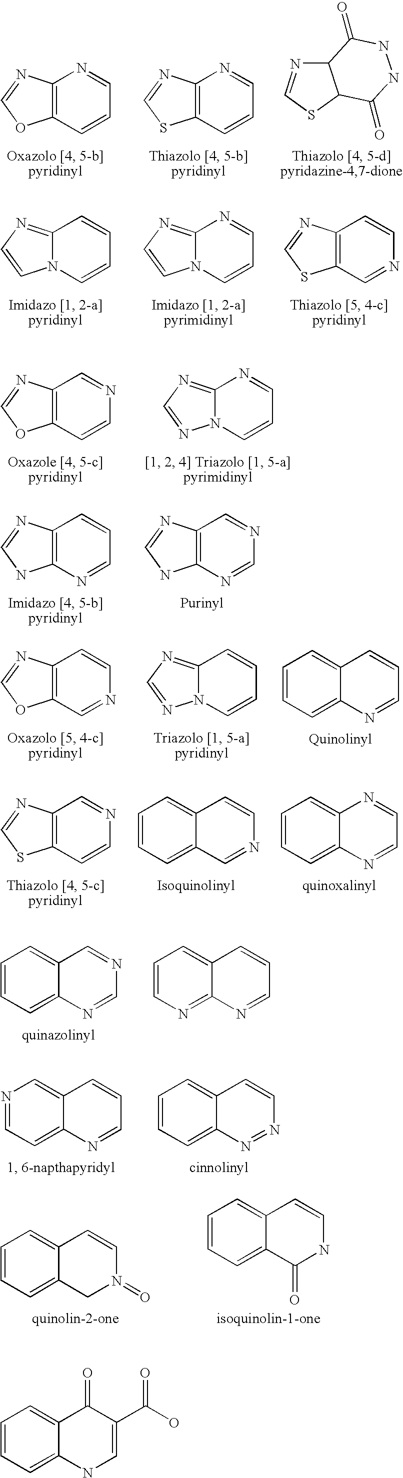

6. A compound of formula I as claimed in claim 1 or a pharmaceutically acceptable salt thereof and wherein R³ is any one of H, F, OCH₃, CH₃, CF₃, CHF₂, CN, CH₂OCH₂CH₃, CONH₂, COOH, Cl, COCH₃

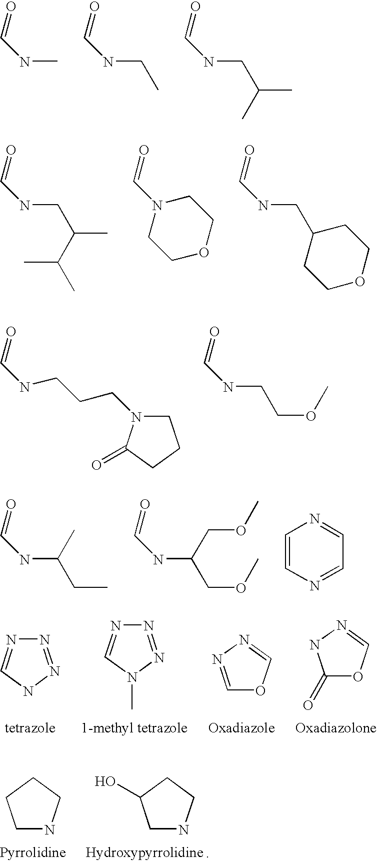

7. A compound of formula I as claimed in claim 1 or a pharmaceutically acceptable salt thereof and wherein R² is any one of H, CH3, OCH3, OCH₂CH₃, OCF₃, OCH₂CH2═CH₂, OCH₂CF₃

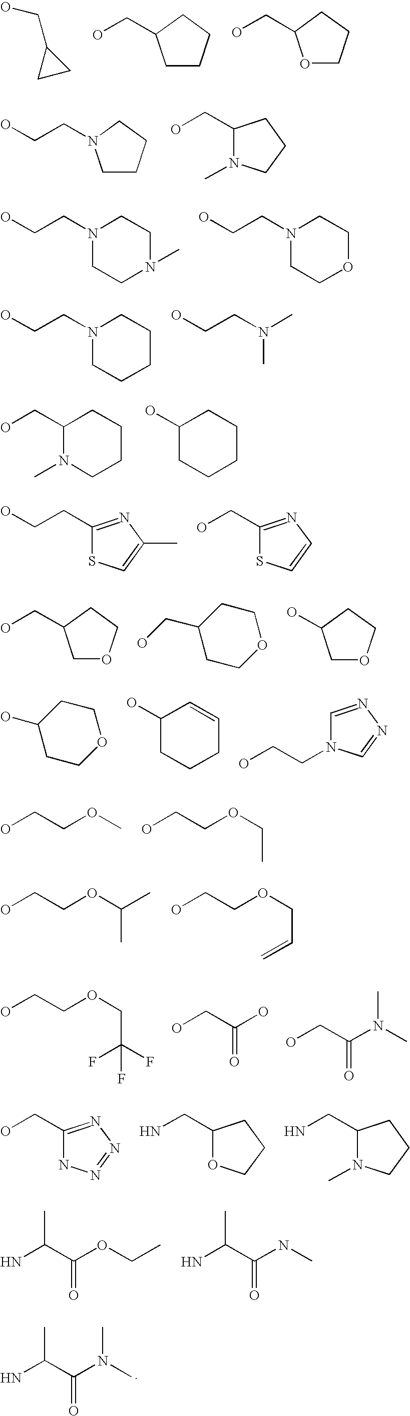

8. A compound of Formula I as claimed in claim 1 or a pharmaceutically acceptable salt thereof and wherein R² is represented as 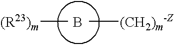
wherein
Z is O, NH, or NCH₃, or Z represents a heterocyclic ring system comprising up to 7 ring atoms and up to 3 heteroatoms each independently selected from N, O and S,
alternatively Z is absent and the R² group is directly attached to the thiazolopyridine or oxazolopyridine ring at the C6 position,
Ring B is selected from one of 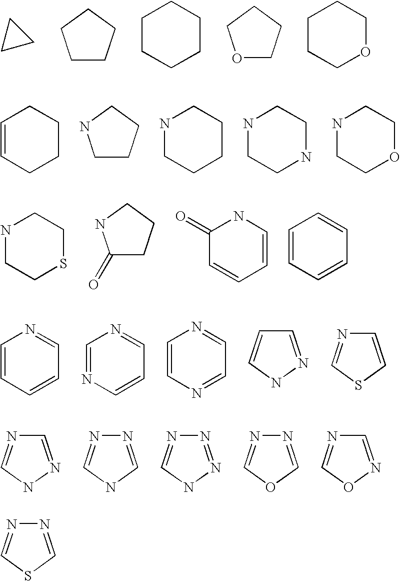
R²³ is H, F, OCH₃, OC2H5, OC(CH3)2, OCH2CH═CH2, OCH2CF3, CH₃, CF₃, CHF₂, CH₂OCH₂CH₃, CONH₂, COOH, Cl, COCH₃

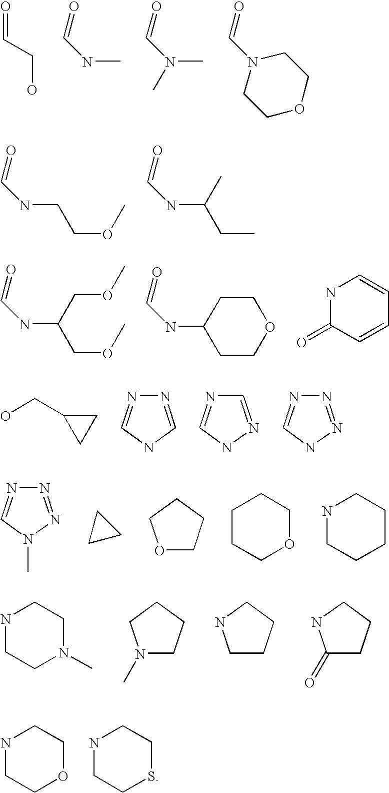

9. A process for preparing a compound of formula I or a pharmaceutically acceptable salt thereof as claimed in claim 1, which process comprises:
a) reacting an amine of the formula (IIa) or (IIb): 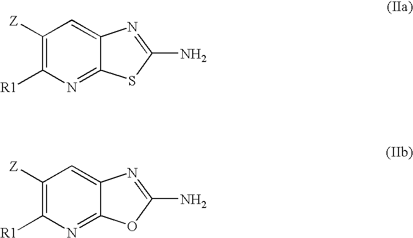
wherein Z is halogen and R1 has the meaning stated in claim 1
with an isocyanate of formula (IIIa) or an activated derivative of formula (IIIb) 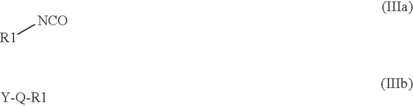
wherein Y is a displaceable group and R1 and Q have the meanings stated in claim 1, in the presence of a suitable base and solvent to give a compound of formula (IVa) or (IVb) 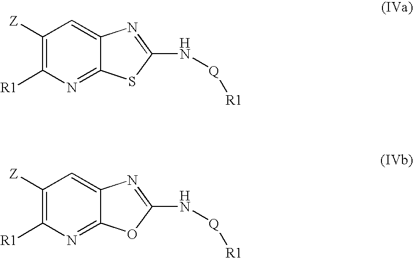
b) reacting boronic acid or boronate ester of the formula (V) 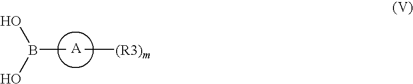
wherein R³, A, R⁷, n and m are as defined in relation to Formula I,
with a compound of formula (IVa) or (IVb) as shown above n the presence of a suitable palladium (0) catalyst to give a compound of formula I as shown above
and
after process a) or b) above, if required doing one or more of the following:
i) converting a compound of the formula (I) into another compound of the formula (I);
ii) removing any protecting groups;
iii) forming a pharmaceutically acceptable salt.

10. A compound of the formula I as claimed in claim 1 or a pharmaceutically-acceptable salt thereof for use in a method of treatment of the human or animal body by therapy.

11. Use of a compound of the formula I as claimed in claim 1 or a pharmaceutically-acceptable salt thereof in the preparation of a medicament for the treatment of the human or animal body by therapy.

12. A method of treating a bacterial infection in an animal in need of such treatment which comprises administering to said animal an effective amount of a compound of formula (I) or a pharmaceutically acceptable salt thereof as defined in claim 1.

13. A pharmaceutical composition comprising a compound of formula (I) or a pharmaceutically acceptable salt thereof as defined in claim 1 together with a pharmaceutically acceptable diluent or carrier.
